(19) United States
(12) Patent Application Publication
(10) Pub. No.: US 2024/0358678 A1
(43) Pub. Date: Oct. 31, 2024

(54) METHODS AND COMPOSITIONS FOR ENHANCING CHEMOTHERAPEUTIC DRUG UPTAKE INTO BRAIN TUMORS

(71) Applicant: Brown University, Providence, RI (US)

(72) Inventors: Sean Lawler, Scituate, MA (US); Jorge-Luis Jimenez-Macias, Providence, RI (US)

(21) Appl. No.: 18/634,563

(22) Filed: Apr. 12, 2024

Related U.S. Application Data
(60) Provisional application No. 63/495,992, filed on Apr. 13, 2023.

(51) Int. Cl. A61K 31/404; A61K 33/243; A61P 35/00

(52) U.S. Cl. CPC A61K 31/404; A61K 33/243; A61P 35/00

(57) ABSTRACT

The present disclosure provides methods for enhancing the uptake of a chemotherapeutic agent into a brain tumor in a subject by administering indirubin, or a derivative thereof, with a therapeutically effective amount of a chemotherapeutic agent.

### CROSS-REFERENCE TO RELATED APPLICATIONS

This application claims the benefit of priority to United States (US) provisional application No. 63/495,992, filed Apr. 13, 2023, the disclosure of which is incorporated by reference as if fully set forth herein in its entirety.

### STATEMENT REGARDING FEDERALLY SPONSORED RESEARCH OR DEVELOPMENT

This invention was made with government support under grant number CA166172 awarded by the National Institutes of Health. The government has certain rights in the invention.

### FIELD OF THE INVENTION

The embodiments of the present invention relate generally to methods for enhancing the uptake of a chemotherapeutic agent into a brain tumor in a subject by administering indirubin, or a derivative thereof, with a therapeutically effective amount of a chemotherapeutic agent.

### BRIEF DESCRIPTION OF THE SEQUENCE LISTING

This submission is accompanied by a “Sequence Listing XML” containing SEQ ID NOs: 1-8 and created on Apr. 8, 2024, 8 KB (kilobytes) size, and submitted with the filename: “405505-718001 US.xml”. None of the sequences therein contain less than 10 amino acids in length or less than 10 nucleotides and none are thus mandatorily marked as intentionally skipped sequences under WIPO Sequence software version 2.3.0. The Sequence Listing XML was generated using WIPO Sequence software version 2.3.0, in accordance with 37 CFR §§ 1.831 through 1.835, and is herewith submitted as an XML file, via the USPTO patent electronic filing system, 37 CFR § 1.835(a)(1). The Sequence Listing XML file is incorporated by reference herein in its entirety.

### BACKGROUND OF THE INVENTION

Brain and other nervous system cancer is the 10th leading cause of death for men and women. In the United States, it is estimated that 18,280 adults (10,710 men and 7,570 women) died from primary cancerous brain and CNS tumors in 2022. Worldwide, an estimated 251,329 people died from primary cancerous brain and CNS tumors in 2020. Malignant gliomas also affect children. About 4,170 children under the age of 15 will also be diagnosed with a brain or CNS tumor this year in the United States.¹ In addition to primary brain tumors, there are also secondary brain tumors or brain metastases where the tumor originated somewhere else in the body and spread to the brain. The most common cancers that metastasize to the brain are breast, kidney, and lung cancers, as well as leukemia, lymphoma, and melanoma.

Brain cancers such as glioblastoma are considered some of the most challenging of tumors, with little improvement in patient survival in decades. Drug uptake into the brain is generally highly restricted and is considered one of the major barriers in the effective treatment of brain cancer. Treatment of brain tumors with chemotherapy is limited mostly because of delivery impediments related to the blood-brain barrier (BBB). For gliomas, the most common and aggressive primary brain tumor, treatment includes surgery, radiotherapy, and chemotherapy usually administered orally or intravenously. These routes do not deliver effective concentrations. To complicate matters, chemotherapy is usually a long treatment. Therefore, transient disruption of the BBB is likely insufficient to deliver effective intratumoral concentrations of anticancer drugs.

Accordingly, there is a need for methods and compositions for enhancing the uptake of chemotherapeutic drugs into brain tumors.

### BRIEF SUMMARY OF THE INVENTION

The present invention is based on the observation that indirubin, or a derivative thereof, enhances the uptake drugs into brain tumors by affecting the permeability at the interface of the tumor and bloodstream (the blood tumor barrier—BTB). By having a greater concentration of the chemotherapeutic agent reaching the tumor, therapeutically effective intratumoral concentrations of chemotherapeutic agent can be achieved.

The embodiments of the present invention provide a method for treating a subject afflicted with a malignant brain tumor comprising administering to the subject: (i) a chemotherapeutic agent; and (ii) indirubin or a derivative thereof. The indirubin or a derivative thereof enhances the uptake of the chemotherapeutic agent into the brain tumor. In some embodiments, the indirubin derivative is 6-bromo-indirubin acetoxime (BiA). In some embodiments, the chemotherapeutic agent is selected from carboplatin, carmustine, cisplatin, cyclophosphamide, etoposide, irinotecan, lomustine, methotrexate, procarbazine, temozolomide, and/or vincristine.

In some embodiments, the chemotherapeutic agent is selected from a platelet-derived growth factor receptor (PDGFR) inhibitor, a vascular endothelial growth factor (VEGF) inhibitor, a broad-selectivity kinase inhibitor, a PI3K inhibitor, a GSK3 inhibitor, a Src inhibitor, and/or a Janus kinase inhibitor (JAK inhibitor). In some embodiments, the inhibitor of PDGFR is selected from Imatinib, Axitinib, Cediranib, Foretinib, Imatinib mesylate, Linifanib, Masitinib, Nintedanib, Ponatinib, Sorafenib, Sunitinib malate, AC 710, AP 24534, CP 673451, DMPQ dihydrochloride, JNJ 10198409, KG 5, PD 166285 dihydrochloride, SU 16f, and/or SU 6668. In some embodiments, the inhibitor of VEGFR is selected from Votrient (pazopanib), Sutent (sunitinib), Avastin (bevacizumab), Nexavar (sorafenib), Stivarga (regorafenib), Cabometyx (cabozantinib), Lenvima (lenvatinib), Iclusig (ponatinib), Cometriq (cabozantinib), Zaltrap (ziv-aflibercept), Inlyta (axitinib), Zirabev (bevacizumab), Vegzelma (bevacizumab), Mvasi (bevacizumab), Fotivda (tivozanib), Cyramza (ramucirumab), Caprelsa (vandetanib), and/or Alymsys (bevacizumab). In some embodiments, the broad-selectivity kinase inhibitor is selected from bosutinib, crizotinib, dasatinib, erlotinib, gefitinib, lapatinib, pazopanib, ruxolitinib, sunitinib, and vemurafenib. In some embodiments, the PI3K inhibitor is selected from Buparlisib (BKM120), Zydelig (idelalisib), Piqray (alpelisib), Vijoice (alpelisib), Copiktra (duvelisib), and/or Aliqopa (copanlisib). In some embodiments, the inhibitor of GSK3 inhibitor is selected from elraglusib, Indirubin-3′-oxime (IDR30, 130), (E/Z)-GSK-3β inhibitor 1, 7-bromoindirubin-3-oxime (7B10), WAY-119064, PF-04802367 (PF-367), RGB-286638 free base, Paeoniae Radix Rubra Extract, Alsterpaullone (Alp, 9-Nitropaullone, NSC 705701), SB216763, Laduviglusib (CHIR-99021, CT99021), AT7519, TWS119, Indirubin (NSC 105327), SB415286, CHIR-98014 (CT98014), Tideglusib (NP031112, NP-12), Laduviglusib (CHIR-99021; CT99021), TDZD-8 (NP 01139), Resibufogenin (Bufogenin, Recibufogenin), 5-Bromoindole, LY2090314, AZD1080, 1-Azakenpaullone (1-Akp), BIO (GSK-3 Inhibitor IX, 6-bromoindirubin-3-oxime, 6-Bromoindirubin-3′-oxime, MLS 2052), AZD2858, AR-A014418, IM-12, Bikinin, BIO-acetoxime, CP21R7 (CP21), 9-ING-41, BRD0705, MAZ51, and/or Chonglou Saponin VII. In some embodiments, the inhibitor of Src is selected from Dasatinib, Alsterpaullone, Bosutinib, Herbimycin A, Piceatannol, Saracatinib, Squarunkin A hydrochloride, Tilfrinib, A 419259 trihydrochloride, AZM 475271, JNJ 10198409, KB SRC 4, KX2-391, LCB 03-0110 dihydrochloride, PD 166285 dihydrochloride, PKI 166 hydrochloride, PP1, 1-Naphthyl PP1, PP2, PP3, Src 11, SU 6656, TC-S 7003, TL 0259, and/or WH-4-023. In some embodiments, the JAK inhibitor is selected from Jakafi (ruxolitinib), Cibinqo (abrocitinib), Inrebic (fedratinib), Olumiant (baricitinib), Opzelura (ruxolitinib), Rinvoq (upadacitinib), and/or Xeljanz (tofacitinib).

In some embodiments, the chemotherapeutic agent is cisplatin. In some embodiments, the indirubin derivative is BiA and the chemotherapeutic agent is cisplatin.

In some embodiments, the administration of the chemotherapeutic agent is done concomitantly or sequentially with the administration of indirubin or a derivative thereof. In some embodiments, the administration of the chemotherapeutic agent and indirubin or a derivative thereof is done concomitantly or sequentially with one or more of a surgical resection, radiotherapy, antiangiogenic therapy, immune therapy, gamma knife radiosurgery, and symptomatic management with corticosteroids.

In some embodiments, the malignant brain tumor is selected from the group consisting of: astrocytoma, glioblastoma, oligodendrocytoma, pilocytic astrocytoma, diffuse intrinsic pontine glioma, ependymoma, oligo-astrocytoma, oligodendrogliocytoma, optic pathway glioma, and hypothalamic glioma.

In another aspect, the embodiments of the present invention provide a method for enhancing the uptake of a chemotherapeutic agent into a brain tumor in a subject in need thereof, the method comprising administering to the subject: (i) a chemotherapeutic agent; and (ii) indirubin or a derivative thereof. The indirubin or a derivative thereof enhances the uptake of the chemotherapeutic agent into the brain tumor. In some embodiments, the indirubin derivative is 6-bromo-indirubin acetoxime (BiA). In some embodiments, the chemotherapeutic agent is carboplatin, carmustine, cisplatin, cyclophosphamide, etoposide, irinotecan, lomustine, methotrexate, procarbazine, temozolomide, or vincristine. In some embodiments, the chemotherapeutic agent is cisplatin. In some embodiments, the indirubin derivative is BiA and the chemotherapeutic agent is cisplatin.

In some embodiments, the administration of the chemotherapeutic agent is done concomitantly or sequentially with the administration of indirubin or a derivative thereof. In some embodiments, the administration of the chemotherapeutic agent and indirubin or a derivative thereof is done concomitantly or sequentially with one or more of a surgical resection, radiotherapy, antiangiogenic therapy, immune therapy, gamma knife radiosurgery, and symptomatic management with corticosteroids.

In some embodiments, the malignant brain tumor is selected from the group consisting of: astrocytoma, glioblastoma, oligodendrocytoma, pilocytic astrocytoma, diffuse intrinsic pontine glioma, ependymoma, oligo-astrocytoma, oligodendrogliocytoma, optic pathway glioma, and hypothalamic glioma.

Other implementations are also described and recited herein.

### BRIEF DESCRIPTION OF THE DRAWINGS

For the purpose of illustration, certain embodiments of the present invention are shown in the drawings described below. It should be understood, however, that the invention is not limited to the precise arrangements, dimensions, and instruments shown. In the drawings:

FIG. 1A provides the chemical structure of indirubin, and the chemical structure of BiA is shown in FIG. 1B.

FIG. 2 shows the dynamic contrast-enhanced magnetic resonance imaging (DCE-MRI) analysis of blood flow within a murine glioblastoma. The left panels show post-contrast MRI scan using gadolinium to outline the tumor area. The right panes show calculated heatmaps of K-trans value which shows increased perfusion 24 hours after BiA treatment (10 mg/kg via IP injection).

FIG. 3A, FIG. 3B, and FIG. 3C depicts the effects of BIA administration on platinum uptake in healthy brain and GBM-bearing tumor mice. FIG. 3A shows that BiA diminishes electrical impedance of endothelial monolayers. FIG. 3B shows that the administration of BiA (20 mg/kg vial tail vein) significantly increases the uptake of platinum (5 mg/kg cisplatin injected via the tail vein 24 hours post-BiA injection) in tumor tissue of brain-tumor bearing mice, but not in healthy mouse brain. FIG. 3C shows that BiA and cisplatin co-administration significantly extends survival in murine glioblastoma.

FIG. 4 shows alterations to barrier functions with RNAseq of human brain microvascular cells (HBMECs) in vitro. Major pathways identified by gene ontology are shown.

FIG. 5 shows an example core chemical structure of indirubins discussed herein including positions of R¹, R², R³, R⁴, R⁵, and R⁶, with non-limiting examples of IRDs provided in Table 2.

FIG. 6 shows Table 2.

FIG. 7 shows a chemical structure of N-Methylisoindigotin (meisoindigo).

FIG. 8 shows a chemical structure of 7-Azaindirubin-3′-oxime.

FIG. 9A shows an example workflow of identification and filtering of genes associated with tumoral vasculature in GBM. Using a gene expression correlation tool, cBIO, top-50 genes that co-expressed with CD31, VWF, CD34 and CLECI4A were selected, confirmed their over-expression in tumor above normal brain in the Rembrandt dataset using GlioVis visualization tool, and evaluated regional expression using the IVY GAP atlas resource. FIG. 9B shows gene expression of 12 tumor endothelium-associated genes (BTB-genes) identified in the cBio Portal following the workflow shown in FIG. 9A, ***p-value<0.001 by Tukey's Honest Significant Difference test. Individual values are colored by GBM subtype Classical, Mesenchymal or Proneural, NA indicates unknown sample information. FIG. 9C shows regional expression of the 12 BTB-genes in GBM using the IVY GAP resource, ***p-value<0.001 by Tukey's Honest Significant Difference test. FIG. 9D shows gene ontology analysis (GO Biological Process 2023) of the 12 BTB-genes using the EnrichR software. Biological processes are ranked by p-values, which are indicated next to the GO designation. FIG. 9E provides a STRING network analysis on the 13 identified BTB-genes. 12 nodes, 17 edges, average node degree 2.83. PPI enrichment p-value 1e\hat{ }-16.

FIG. 10A shows a surface plot of CDH5 expression from spatial transcriptomic performed on GBM tumors and non-tumorigenic cortex. FIG. 10B shows a spatial plot of CDH5 expression in cortex and tumor tissue from patient UKF_“248”. FIG. 10C shows a UMAP plot from cortex and tumor indicating spatial CDH5 expression from FIG. 10B and, FIG. 10D shows integrated-clustered UMAP of the cortex and tumor transcriptional profiles (left) and DimPlot of CDH5 expression (right). FIG. 10E provides violin plots showing CDH5 and endothelial markers clustered-expression levels. FIG. 10F illustrates gene ontology biological processes enriched in the CDH5-expressing clusters, indicating vasculature development and regulation of angiogenesis as pathways involved. Data was obtained and re-analyzed from Ravi et al., (2022) using the SPATA2 package from R-studio.

FIG. 11A shows a heat-map generated from the top-15 upregulated and downregulated genes from Bulk-RNA sequencing analysis performed on HCMEC/D3 cells treated with BIA (1 μM, 24 hrs). FIG. 11B shows a gene ontology analysis of >1.5 significantly upregulated and downregulated genes (Log 2 fold-change) by BIA in brain endothelial cells from FIG. 11A. Biological processes are ranked by p-values, which are shown next to the GO designation. Analysis performed using the EnrichR software. FIG. 11C shows a volcano plot analysis from all the upregulated and downregulated genes by BIA. Labels on genes related to angiogenesis, TGF-β and WNT pathways are highlighted. FIG. 11D shows gene expression fold-change (Log 2) levels of dysregulated genes by BIA related to the TGF-β and WNT pathways, angiogenesis and the tumor vascular associated genes (BTB-genes, highlighted). FIG. 11E provides violin plots displaying expression levels of genes related to Angiogenesis, TGF-β and WNT pathways in sample UKF_248 which expression or activity are modulated by BIA. FIG. 11F shows surface plots of genes related to angiogenesis, TGF-β and WNT pathways in sample UKF_334.

FIG. 12A shows immunofluorescence staining of CDH5 with Alexa Fluor 594 (red) and nuclei with Hoechst 33342 (blue) in HCMEC/D3 cells treated with BIA (1 μM, 24 hrs). Representative image shown at 20×, scale bar=20 μm. FIG. 12B shows TEER assay resistance values of HCMEC/D3 treated with BIA upon monolayer confluence (capacitance ˜10 nF). Time-point of BIA addition is indicated (˜110 hrs.). FIG. 12C shows confocal microscopy immunofluorescence images of BBB spheroids treated with indicated doses of BIA for 72 hours. Staining of F-actin with Phalloidin-Alexa Fluor 488 (green), CDH5 with Alexa-Fluor 594 (red) and nuclei with Hoechst 33342 (blue). Maximal projection intensity is shown from z-stack images (5 μm depth, 20 layers). Pictures taken at 10×, scale bar=100 μm. FIG. 12D shows FITC-conjugated dextran (70 kDa) permeability assay in BBB spheroids. Dextran (gray) and nuclei stained with Hoechst 33342 (purple or brighter grayscale at center and right columns) are shown. Maximal projection intensity is shown from z-stack images (5 μm thick, 20 layers). Pictures taken at 10×, scale bar=100 μm. Mean fluorescence quantification of nuclei is shown in FIG. 12E CDH5, in FIG. 12F Phalloidin, and in FIG. 12G (FITC-dextran) from the images in FIG. 12C and FIG. 12D using the Image J (Fiji) software. Data shows individual values, mean and standard deviation, n=4-5. Ordinary one-way ANOVA for statistical significance. **p=0.0028, ***p=0.008, ****p<0.0001. FIG. 12H shows human phospho-kinase array of HCMEC/D3 cells exposed to 1 uM BIA for 24 hours. Colored squares (or different shaded squares) highlight wells related to the indicated pathways. Samples were analyzed in duplicates. FIG. 12I shows quantification of signal by Image J (Fiji) of dot-blot shown in FIG. 12H. Mean and standard deviation of duplicates are shown. Two-way ANOVA analysis was performed. **p=0.0015, ***p=0.005, ****p<0.0001.

FIG. 13A shows an example workflow schematic of BIA administration and subsequent injection of NaF for BTB permeability assessment. FIG. 13B shows in vivo imaging system (IVIS) pictures of G30-tumor bearing brains from mice injected with BIA and NaF as shown in FIG. 13A. FIG. 13C shows quantification of image intensity, that was performed with Image J (Fiji). Mean and standard deviation are shown, n=7-8. Unpaired t-test for statistical significance, **p=0.0024. FIG. 13D shows platinum quantification via ICP-MS of brain and tumor tissue from tumor-bearing mice injected with cisplatin in G9-PCDH, FIG. 13E shows G34-PCDH, and FIG. 13F shows GL-261 murine models. Cisplatin (5 mg/kg) was administered 24 hours after BIA injection. Mean and standard deviation are shown, n=3-5/group. Two-way ANOVA statistical analysis was performed, *p<0.05. FIG. 13G shows platinum quantification via ICP-MS of tumor and brain tissue of a G9-PCDH tumor-bearing xenograft model administered with increasing BIA doses. Mean and standard deviation are shown, n=3/group. Two-way ANOVA test, *p=0.0177, **p=0.0016. FIG. 13H shows confocal immunofluorescence imaging from frozen and sectioned brain tissue from G9-PCDH and G34-PCDH xenograft murine models, 24 hours after injection with 20 mg/kg of BIA. CDH5 was stained in tumor and healthy brain tissue with Alexa Fluor 594 and blood-vessels with anti-CD31 and Alexa Fluor 405. GBM cells are pre-labelled with GFP. Images shown at 20×, with scale bars at 100 μm, accordingly. FIG. 13I shows CDH5 fluorescence quantification (Alexa Fluor 594) from experiment in FIG. 13E using Image J (Fiji). Mean and standard deviation are shown. Unpaired t-test (n=3/group). **p=0.0013, *p=0.0334.

FIG. 14A shows a GBM cell viability assay (ATP-based) using Cell-Titer Glo 3D of BIA and cisplatin combination treatment. Cisplatin doses are indicated in x-axis, BIA remained at a constant concentration of 1 μM. Cells were treated for 5 days and analyzed using a plate reader for luminescence quantification. Mean and standard deviation are shown, n=3/group. Two-way ANOVA test for significance. *p=0.028, **p=0.0096, ***p=0.0003, **** p<0.0001. FIG. 14B shows immunofluorescence staining of γH2AX (Alexa-Fluor 647) in G9-PCDH cells treated with 1 μM of cisplatin and/or BIA, for 72 hours. Nuclei were stained using Hoechst 33342. Representative image of 5 pictures per condition. Pictures taken at 40×, scale bar=20 μm. FIG. 14C shows quantification of γH2AX foci from FIG. 14B using Image J. FIG. 14D shows a western blot of G9-PCDH cells treated with 1 μM of cisplatin and/or BIA, for 72 hours, probing for the ATR/CHK1 axis proteins. GAPDH was used as loading control and cleaved-PARP as a cell-death marker. Representative image from triplicate experiments. FIG. 14E shows a dose-response matrix showing inhibition percentage of BIA and cisplatin combinations at various concentrations using SynergyFinder 3.0. G9-PCDH cells were treated and viability analyzed as indicated in the Cell viability assay section (see Materials and methods). FIG. 14F shows a ZIP method synergy score of BIA and cisplatin combinations. The overall average 5-score is indicated on top of the chart. The dose combinations showing an increased likelihood of synergy is highlighted.

FIG. 15A and FIG. 15C show diagrams of the experimental design for G34-PCDH and G9-PCDH xenograft efficacy studies using BIA/PPRX-1701 and cisplatin combinations. FIG. 15B shows efficacy studies of G34-PCDH xenograft using BIA and FIG. 15D shows PPRX-1701 in combination with cisplatin. For PPRX-1701 studies, empty nanoparticles were used as controls and in combination with cisplatin. N=8/group. Log-rank test analysis for statistical significance. FIG. 15E shows confocal immunofluorescence imaging of γH2AX (Alexa Fluor 647, red) nuclear foci from tumor tissue collected from study (D). Nuclei were stained with Hoechst 33342 (blue). Representative pictures taken at 20×. Scale bar=50 μm. FIG. 15F shows a quantification of γH2AX foci from FIG. 15E using Image J, n=6/group. Ordinary One-way ANOVA was performed for statistical evaluation. *p=0.01, **p=0.0086. FIG. 15G shows a schematic of proposed model of BIA/PPRX-1701 mechanism of action and its effects in GBM tumor drug delivery and anti-oncogenicity.

FIG. 16A shows a surface plot of CDH5 expression (top), clustered UMAP (middle), and violin-plot showing clustered CDH5 expression levels (bottom) of sample UKF_242, FIG. 16B shows sample 313 and FIG. 16C shows sample 334. FIG. 16D shows CDH5 and endothelial-cell marker expression levels in clusters of sample 248, FIG. 16E shows sample 259 and FIG. 16F shows data for sample 334.

FIG. 17A shows IF staining of CDH5, ZO-1 and Claudin-5 in HCMEC/D3 cells treated with BIA (1 μM, 48 hours). Representative image is shown. Scale bar=100 μm. FIG. 17B shows real-time PCR screening for CDH5 in HBMEC and HCMEC/D3 cells treated with 1 μM BIA for the indicated time-points. Mean and standard deviation are shown. Ordinary One-way ANOVA test *p=0.016, ****p<0.0001. FIG. 17C shows real-time PCR screening for CDH5, S1 PR3 and WNT7B in G34-PCDH neurosphere GBM cells treated with BIA (2 μM) for 24 hours. Mean and standard deviation are shown. Multiple unpaired t-test was performed, * p<0.05. FIG. 17D shows western blot analysis of CDH5 expression at different timepoints in HCMEC/D3 cells exposed to 1 μM BIA. Actin was used as loading control. FIG. 17E shows TEER analysis of HCMEC/D3 and HBMEC cells treated with BIA 24 hours after plating. Mean and standard deviation values are provided. FIG. 17F shows HCMEC/D3 dose response to different BIA doses analyzed by TEER. Two-way ANOVA test for control vs. BIA treatment comparisons (n=2/group), ****p<0.0001.

FIG. 18A shows a flow cytometry analysis of late apoptosis by Sytox Blue staining in HCMEC/D3 treated with different doses of BIA or Cisplatin (5 μM) for 72 hours. FIG. 18B shows a cell titer Glo viability assay on HCMEC/D3 and HBMEC cells treated with increasing concentrations of BIA for 72 hours. Mean and standard deviation of relative viability to control are shown, n=3 per group. FIG. 18C provides brightfield images of HCMEC/D3 cells treated with BIA for 72 hours. Scale bar=50 μm. FIG. 18D shows cell cycle analysis via flow cytometry of HCMEC/D3 cells treated with BIA (0.5 μM or 1 μM) or DMSO control for 72 hours. Representative figure of three-independent experiments is shown.

FIG. 19A shows a heatmap of Luminex cytokine analysis of HCMEC/D3 cells treated with 1 μM for 48 hours. FIG. 19A provides fold-change relative to DMSO controls shows upregulated cytokines, and FIG. 19B shows fold-change decrease of cytokine secretion upon BIA doses.

FIG. 20A shows a scheme of experimental layout for intra-cranial GBM tumor implantation in nude mice and subsequent treatment with BIA and cisplatin for ICP-MS quantification. FIG. 20B shows platinum (Pt195) quantification in peripheral organs of cisplatin injected mice, 24 hours after BIA administration. Two-way ANOVA statistical analysis was performed from triplicate samples. FIG. 20C shows platinum levels in G9-PCDH and G34-PCDH and FIG. 20D shows HCMEC/D3 cells after treated with 2 μM BIA and cisplatin for the indicated exposure times. Mean and standard deviation are shown from triplicates. Two-way ANOVA statistical analysis was performed. FIG. 20E shows a western blot from HCMEC/D3 cells treated with BIA at (1 μM) for 48 hours. Actin was used as loading control. FIG. 20F shows IF staining of ZO-1 and Claudin-5 in the G9-PCDH xenograft model. Mice were treated with 20 mg/kg of BIA, and 24 hours later, brains were collected and fixed for sectioning and staining. Images taken at 20×.

FIG. 21A shows growth-in-low-attachment (GILA) assay for neurosphere formation assessment in FIG. 21A is G9-PCDH and FIG. 21B shows G34-PCDH cell lines. Neurosphere size was measured using Image J (Fiji) in FIG. 21C and in FIG. 21D for G9-PCDH and G34-PCDH, respectively. FIG. 21E shows Flow cytometry analysis of cell cycle by propidium iodide (PI) and DNA damage by γH2AX (Ser139) staining in G62 cells treated with BIA (1 μM) or cisplatin (1 μM) and their combination for 72 hours. Representative figure of two independent experiments is shown. FIG. 21F shows a graph of proportions of different cycle stages from data shown in FIG. 21E. FIG. 21G shows depletion of CHK1 via siRNA in G9-PCDH and G30 cells. Cells were then treated with different doses of cisplatin doses for 96 hours. Cell viability is shown relative to non-treated controls. Western blot confirmation of depletion is shown, with GAPDH as loading control. Mean and standard deviation are shown.

FIG. 22A shows luminescence detection of G9-TCF reporter cell line treated with increasing concentrations of BIA or PPRX-1701 nanoparticles for 5 hours. Luminescent signal was quantified by IVIS and shown in the graph to the right. No significant differences were found by Two-way ANOVA test, n=3. FIG. 22B shows BIA quantification in tumor and non-tumor cortex tissues from the G30-LRP xenograft murine model injected with either BIA or PPRX-1701 (20 mg/kg). Quantification was performed using LC-HRMS. Mean and standard deviation are shown. No significant differences were seen by Two-way ANOVA statistical test.

FIG. 23 shows Supplementary Table 1 co-expressed genes with PECAM1, CD34, VWF and CLEC14A in TCGA Firehose Legacy analyzed with cBIO.

FIG. 24 shows Supplementary Table 2 genes co-expressed with CDH5 in GBM patient-samples from the spatial transcriptomics analysis in Ravi, et al., (2022).

FIG. 25 shows Supplementary Table 3 a table of primers used for real-time PCR analyses, including forward primer sequences GGTCGATGCAGAGACAGGAG (SEQ ID NO: 1), CCAGCAAGAGCACAAGAGGA (SEQ ID NO: 2), GACTGCTCTACCATCCTGCC (SEQ. ID NO: 3), GCGCTCGTCTCCGTCTATT (SEQ ID NO: 4); and reverse primer sequences GAGTCTCCAGGTTTCGCCA (SEQ ID NO: 5), ACATGGCAACTGTGAGGAGG (SEQ ID NO: 6), GATGCGTGCGTAGAGGATCA (SEQ ID NO: 7), and AGATGATGTTGGCTCCCAGG (SEQ ID NO: 8).

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as brain cancer. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical parameters are improved. Alternatively, treatment is “effective” if the progression of a condition is reduced or halted. That is, “treatment” includes not just the improvement of symptoms or clinical parameters, but also a cessation or at least slowing down of progression or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing down of tumor growth and/or metastasis, and an increased lifespan as compared to that expected in the absence of treatment.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intra-lymphatic, intra-lymph node, intravenous, intraportal vein, intrahepatic arterial, intramuscular, intraarterial, intrathecal, intracapsular, intraorbital, intratumoral, intracardiac, intradermal, intraperitoneal, intranasal, intratracheal, intrathecal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

### Cancer-Related Definitions:

As used herein, the term “cancer” relates generally to a class of diseases or conditions in which abnormal cells divide out of control and can migrate and invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor.

As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form solid tumors, but some, e.g., leukemias, do not necessarily form solid tumors. For those cancer cells that form solid tumors, the terms cancer (cell) and tumor (cell) are used interchangeably.

A subject that has a cancer or a tumor is a subject having objectively measurable cancer cells present in the subject's body. Included in this definition are malignant, actively proliferative cancers, as well as potentially dormant tumors or micrometastatses. Cancers which migrate away from their original location, invade and seed other vital organs can eventually lead to the death of the subject through the functional deterioration of the affected organs. Hemopoietic cancers, such as leukemias, are able to out-compete the normal hemopoietic compartments in a subject, thereby leading to hemopoietic failure (in the form of anemia, thrombocytopenia and neutropenia) ultimately causing death.

Examples of cancer include but are not limited to, adenocarcinoma; blastoma, such as neuroblastoma; carcinoma, such as basal cell carcinoma; biliary tract cancer; bladder cancer; bone cancer; brain and CNS cancer, such as glioblastoma multiforme (GBM); breast cancer; cervical cancer; choriocarcinoma; colon and rectum cancer; connective tissue cancer; cancer of the digestive system; endometrial cancer; esophageal cancer; eye cancer, such as retinoblastoma; cancer of the head and neck; gastric/stomach cancer (including gastrointestinal cancer); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; oral cavity cancer (e.g., lip, tongue, mouth, and pharynx); ovarian cancer; pancreatic cancer; cancer of the peritoneum; prostate cancer; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, malignant or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition or density limitation of growth, growth factor or serum independence, upregulated/highly expressed tumor specific markers, invasiveness or metastasis, and tumor growth in suitable animal hosts such as immunocompromised, immunodeficient or even immunocompetent (for mouse-derived cancer) mice.

A subject can be one who has been previously diagnosed with or identified as suffering from or having a condition in need of treatment (e.g., a cancer) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive corn, sunflower, grapeseed, vegetable, fish oil, or injectable organic esters. In preferred embodiments, when such pharmaceutical compositions are for human administration, particularly for invasive routes of administration (i.e., routes, such as injection, pump infusion, transplantation or implantation, that circumvent transport or diffusion through an epithelial barrier), the aqueous solution is pyrogen-free, or substantially pyrogen-free. The excipients can be chosen, for example, to effectuate delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, gel, spray, aerosol (e.g., pressurized intraperitoneal aerosol), radioactive isotope, intervention (e.g., trans-arterial embolization, radiofrequency ablation), injection, infusion or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, decrease degradation, rejection or clearance, increase solubility, increase immunogenicity, or to increase the absorption of a compound such as a compound of the invention. Such physiologically acceptable agents include, for example, carbohydrates, such as glucose, sucrose or dextrans; antioxidants, such as ascorbic acid or glutathione; chelating agents; low molecular weight proteins; adjuvants (e.g., Alum, MF59, AS01/03/04, CpG1018) or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, metals, radioactive isotope/radiation particles, and/or dosage forms which are, within the scope of sound medical judgment, suitable for use in contact with the tissues of human beings and animals without excessive toxicity, irritation, allergic response, rejection, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, vegetable oil, cottonseed oil, safflower oil, sesame oil, olive oil, sunflower oil, grapeseed oil, fish oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, matrigel or hydrogel; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) nanoparticles such as liposomes, polymers, micelles, metal nanoparticles, carbon nanotubes, solid lipid nanoparticles, noisomes, and dendrimers; (22) extracellular vesicles; and (23) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, pills, suppository, patch, paste, infusion pumps, capsules (including sprinkle capsules and gelatin capsules), boluses, powders, granules, pastes for application to the tongue); absorption through the oral, nasal, urinary, rectal or vaginal mucosa (e.g., sublingually, aerosol); subcutaneously; intradermally, transdermally (for example as a patch applied to the skin); intramuscularly; intravenously; and topically (for example, as a cream, lotion, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration, capsules, including sprinkle capsules and gelatin capsules, (coated and uncoated, bi-layer, mini-) tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as carboxymethylcellulose, alginates, gelatin, polyvinyl pyrrolidone, sucrose and/or acacia; (3) humectants, such as glycerol; (4) disintegrating agents, such as agar-agar, gel, hydrogel, matrigel, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as modified and unmodified cyclodextrins; (11) flavoring agents; (12) thermoregulation (cooling or heating) agents; and (13) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, nanoparticles such as liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the respiratory, urogenital, gastrointestinal tract, optionally, in a delayed manner. Examples of embedding compositions that can be used include polymeric substances and waxes. The active ingredient can also be in micro-encapsulated form, extracellular vesicle form, if appropriate, with one or more of the above-described excipients.

Liquid dosage forms useful for oral administration include pharmaceutically acceptable emulsions, lyophiles for reconstitution, micro-emulsions, microcapsules, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., fish, cottonseed, groundnut, corn, germ, vegetable, grapeseed, sunflower, olive, castor and sesame), glycerol, tetrahydrofurfuryl alcohol, polyethylene glycols and fatty acid esters of sorbitan, and mixtures thereof.

Besides inert diluents, the oral compositions can also include adjuvants such as Alum, MF59, AS01/03/04, CpG1018, wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, creams, lotions, ointments, gels, hydrogel, matrigel, solutions, patches, pastes, microneedles (for transdermal, intraocular, vaginal, transungual, cardiac, vascular, gastrointestinal and intracochlear delivery) and inhalants. The active compound may be mixed under sterile conditions with a pharmaceutically acceptable carrier, adjuvant, and with any preservatives, buffers, or propellants that may be required.

The ointments, pastes, creams, lotions, patches, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intra-articular, intra-lymph node, intra-lymphatic, intratumoral, intrathecal, intracapsular, intraorbital, intracardiac, intradermal, intracochlear, intraperitoneal, intravaginal, transdermal, transtracheal, transungual, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity (flow/adhesion ratio) can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial, antiviral, and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in nanoparticles such as liposomes, extracellular vesicles, microencapsulations or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically-acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion or clearance of the particular compound(s) being employed, the duration or frequency of the treatment, other drugs that may interact with or affect the metabolism or efficacy of the compound of the invention, compounds and/or materials (e.g., vaccines, antibodies and derivatives) used in combination with the particular compound(s) employed, other therapeutic approaches (e.g., surgery, cell-based therapy, chemotherapy, radiotherapy, interventional therapy), the age, sex, body weight, conditions or comorbidities, diet, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the body weight, sex, age, comorbidities, and medical history of the subject. Other factors which influence the effective amount may include, but are not limited to, the severity of the patient's condition, the disorder being treated, therapeutic approaches, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher et al. (1996).²

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent or combined with a therapeutic approach (e.g., surgery, chemotherapy, radiotherapy, interventional therapy).

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers, dispersants and lubricants, such as sodium lauryl sulfate and magnesium stearate, as well as coloring agents, release agents, coating agents, sweetening, cooling or heating agents, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, polyunsaturated fatty acids (PUFAs) such as Omega-3 fatty acids, and the like; and (3) metal-chelating agents, such as citric acid, ethylenediamine tetraacetic acid (EDTA), sorbitol, tartaric acid, phosphoric acid, succimer (dimercaptonol), dimercaprol (BAL), and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, animal models, (engineered or genetically modified) cells, organoids, constructs, vectors, carriers, adjuvants, compounds, drug delivery system, antibodies and derivatives, vaccines, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;³ The Encyclopedia of Molecular Cell Biology and Molecular Medicine;⁴ Molecular Biology and Biotechnology: a Comprehensive Desk Reference;⁵ Immunology;⁶ Janeway's Immunobiology;⁷ Lewin's Genes XI;⁸ Molecular Cloning: A Laboratory Manual.;⁹ Basic Methods in Molecular Biology;¹⁰ Laboratory Methods in Enzymology;¹¹ Current Protocols in Molecular Biology (CPMB);¹² Current Protocols in Protein Science (CPPS);¹³ and Current Protocols in Immunology (CPI).¹⁴

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Gliomas

Glial-derived tumors (i.e., gliomas) are a common type of tumor originating in the brain rather than a metastatic brain cancer. About 33% of all brain tumors are gliomas, which originate in the glial cells that surround and support neurons in the brain, including astrocytes, oligodendrocytes and ependymal cells. Gliomas are transformed cells that display increased metabolic activity as a result of the transformation process. They comprise a diverse group of neoplasms that differ in their morphology, their CNS location, their degree of invasiveness, their tendency for progression, and their growth characteristics. Neoplastic transformation can occur in all glial cell types, thereby producing a large range of pathological and morphological variants. Most primary brain tumors derived from glial cells have lost growth control regulation, giving rise to astrocytomas, glioblastomas, or oligodendrocytomas. In the 2016 World Health classification of brain tumors, there were no less than 36 different subtypes of glioma identified, based on histology and various biomarkers.¹⁵

Astrocytomas are glial cell tumors developed from connective tissue cells called astrocytes and are the most common primary intra-axial brain tumor, accounting for nearly half of all primary brain tumors. They are most often found in the cerebrum (the large, outer part of the brain), but also in the cerebellum (located at the base of the brain).

Astrocytomas can develop in adults or in children. High-grade astrocytomas, called glioblastoma multiforme (GBM), are the most malignant of all brain tumors. Glioblastoma symptoms are often the same as those of other gliomas. Pilocytic astrocytomas are low-grade cerebellum gliomas commonly found in children. In adults, astrocytomas are more common in the cerebrum.

Brain stem gliomas, also called diffuse infiltrating brainstem gliomas or diffuse intrinsic pontine gliomas (DIPGs), are rare tumors found in the brain stem. They usually cannot be surgically removed because of their remote location, where they intertwine with normal brain tissue and affect the delicate and complex functions this area controls. These tumors occur most often in school-age children where they are responsible for the greatest number of childhood deaths from primary brain tumors.

Ependymomas develop from ependymal cells lining of the ventricles or in the spinal cord. Ependymomas are rare, accounting for just 2% to 3% of primary brain tumors. However, they account for about 8% to 10% of brain tumors in children and are more likely to affect those younger than 10 years old. The most location for ependymomas in children is near the cerebellum, where the tumor can block the flow of the cerebral spinal fluid and cause increased pressure inside the skull (obstructive hydrocephalus). These tumors can spread to other parts of the brain or spinal cord (drop-metastases) due to the flow of spinal fluid.

Mixed gliomas (also called oligo-astrocytomas) are made up of more than one type of glial cell. Their diagnosis as a distinct tumor type is controversial and may be resolved with genetic screening of tumor tissue. These tumors are often found in the cerebrum and are most common in adult men.

Oligodendrogliomas form from oliogodendrocytes, the supportive tissue cells of the brain and are usually found in the cerebrum. About 2% to 4% of primary brain tumors are oliogodendrogliomas. They are most common in young and middle-aged adults and more likely to occur in men. Seizures are a very common symptom of these gliomas (affecting 50% to 80% of patients), as well as headache, weakness, or problems with speech. Oligodendrogliomas typically have a better prognosis than most other gliomas. They are significantly more favorable in terms of their biological behavior and their prognosis for patients and can have survivorship measured of decades for folks with these tumors that have the most favorable biomarker profiles.

Optic pathway gliomas are a type of low-grade tumor found in the optic nerve or chiasm. They often infiltrate the optic nerves, which send messages from the eyes to the brain. People with neurofibromatosis are more likely to develop them. Optic nerve gliomas can cause vision loss and hormone problems, since these tumors are often located at the base of the brain where hormonal control is located. Gliomas affecting hormone function may be known as hypothalamic gliomas.

The most important determinant of survival for gliomas is the “grade” of the glioma. Increasing grades represent increasing malignancy and decreasing differentiation, which is associated with increased mitotic activity and enhanced cell migration.^{16,17} Grade I tumors grow slowly and can sometimes be totally removed by surgery, while grade IV tumors are fast-growing, aggressive, and difficult to treat. High-grade gliomas account for 30% of primary brain tumors in adults and are the second most common cause of cancer death in children under 15 years of age. High-grade gliomas are divided by grade into two categories: anaplastic astrocytomas (WHO Grade Ill) and glioblastoma multiforme (GBM; WHO Grade IV).¹⁸

GBM (Grade IV) tumors are characterized by the presence of areas of necrotizing tissue that are surrounded by anaplastic cells with numerous mitosis and endothelial proliferation. This characteristic, as well as the presence of hyperplastic abnormal blood vessels, differentiates the tumor from Grade Ill astrocytomas, which do not have these features. GBM tumors usually appear in the cerebral white matter, grow quickly, and can become very large before causing symptoms. Less than 10% form more slowly, following dedifferentiation of low-grade astrocytoma or anaplastic astrocytoma. These are called secondary GBM tumors and are more common in younger patients (mean age 45 versus 62 years). The tumor may extend into the meninges or ventricular wall, leading to high protein content in the cerebrospinal fluid (CSF) (>100 mg/dL), as well as an occasional pleocytosis of 10 to 100 cells, mostly lymphocytes. Malignant cells carried in the CSF may spread (rarely) to the spinal cord or cause meningeal gliomatosis. However, metastasis of GBM beyond the central nervous system is extremely unusual. About 50% of GBM tumors occupy more than one lobe of a hemisphere or are bilateral. Tumors of this type usually arise from the cerebrum and may rarely exhibit the classic infiltration across the corpus callosum, producing a butterfly (bilateral) glioma.

GBM has the worst prognosis of any central nervous system (CNS) malignancy, despite multimodality treatment consisting of surgical resection of as much of the tumor as possible, with concomitant or sequential chemotherapy, radiotherapy, antiangiogenic therapy, immune therapy, gamma knife radiosurgery, and symptomatic management with corticosteroids. Prognosis is very poor, with a median survival time of approximately one year and the disease is almost invariably fatal, as only about 3% survive for more than 3 years.

### Indirubin and Derivatives

Indirubin, an indigo dye, is a biomolecule found in the African and Asian shrub Indigofera arrecta (see, Table 1). It is variously known as indigo red; indigo naturalis; qing dai; or Natal, Bengal, or Java indigo. It is also bacterially produced in the urine of humans and other mammals and has been used since 627 AD in traditional Chinese medicine. It is essentially the indigo dye as traditionally extracted from plants by fermentation and lime treatment.¹⁹

TABLE 1
Indirubin

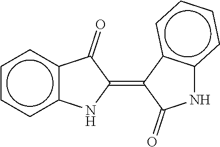

IUPAC name: (3Z)-3-(3-Oxo-1,3-dihydro-2H-indol-2-ylidene)-
1,3-dihydro-2H-indol-2-one
Molecular Formula: C₁₆H₁₀N₂O₂
Synonyms: Indigo Red 479-41-4 Couroupitine B Indigopurpurin

Indirubin derivatives (IRDs), also known as indirubins, are bisindole alkaloids naturally occurring in indigo-bearing plants or in mollusks from the Muricidae family. They belong to the rather small family of indigoids, which has nevertheless found an extreme importance in the fields of dyes and medicinal chemistry. Indirubin has been found to be the active ingredient of a traditional Chinese Medicine used to treat the symptoms of leukemia. Further biological explorations revealed the ability of indirubin to bind cyclin-dependent kinases and 6-bromoindirubin, extracted from mollusks, to bind glycogen synthase kinase-3. The high affinity displayed by the indirubin derivatives has opened a vast field of research and triggered the development of hundreds of derivatives with biological activities.²⁰ Numerous IRDs have been described in the art.²¹⁻²⁷

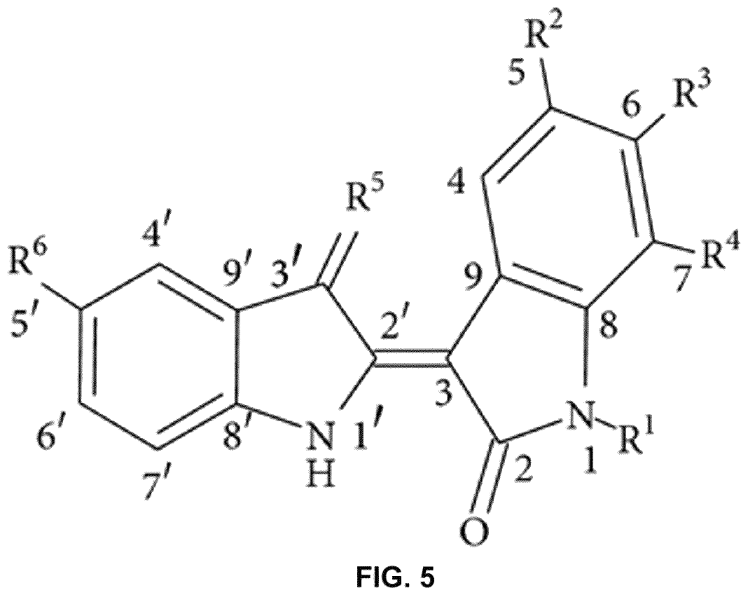

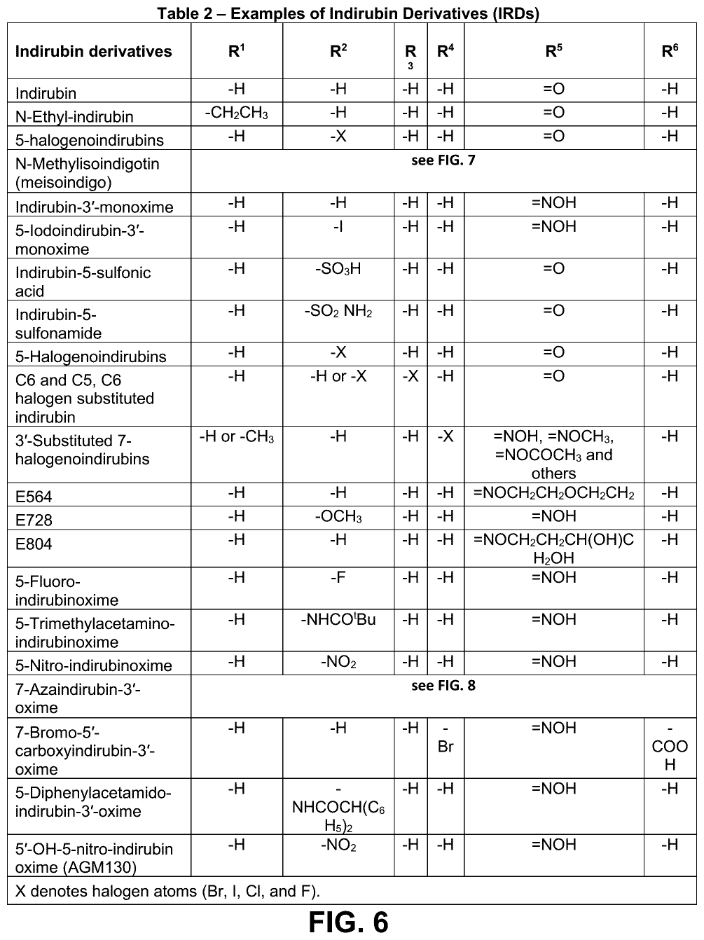

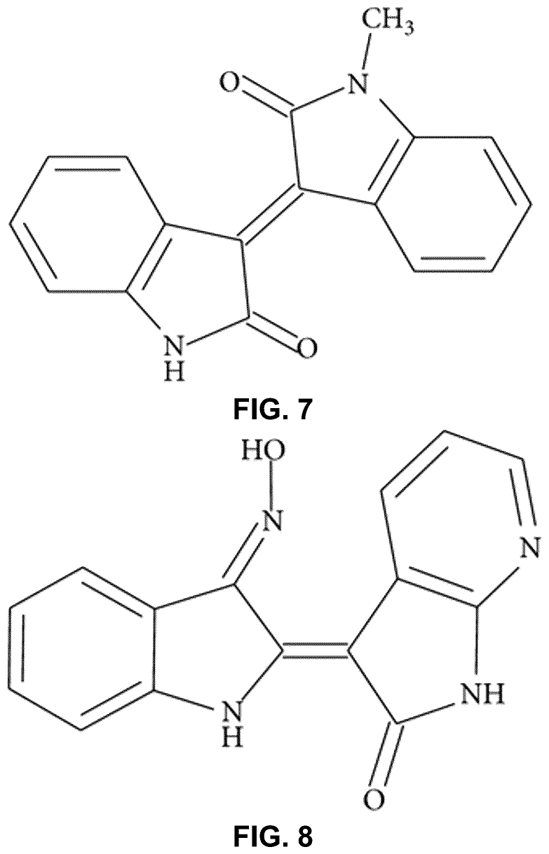

The core structure of indirubins discussed herein is shown in FIG. 5, including positions of R¹, R², R³, R⁴, R⁵, and R⁶, with non-limiting examples of IRDs provided in Table 2 (Examples of Indirubin Derivatives, IRDs); Table 2 is shown in FIG. 6. The chemical structure of N-Methylisoindigotin (meisoindigo) is shown in FIG. 7, and the chemical structure of 7-Azaindirubin-3′-oxime is shown in FIG. 8.

Additional IRDs include, but not limited to, the IRDs provided in Table 3.

TABLE 3
Additional Examples of Indirubin Derivatives (IRDs)

| | |
|---|---|
| 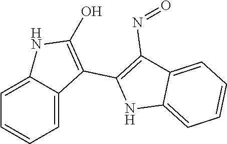 [SMILES: O=Nc1c(-c2c(O)[nH]c3ccccc23)[nH]c2ccccc12] | (A) |
| 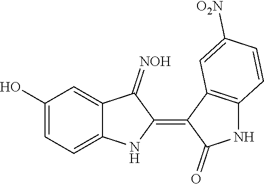 | (B) |
| 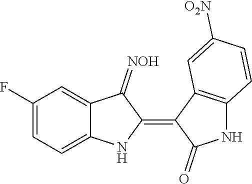 | (C) |
| 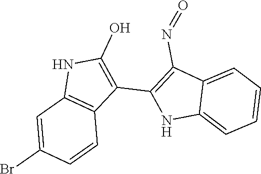 [SMILES: O=Nc1c(-c2c(O)[nH]c3cc(Br)ccc23)[nH]c2ccccc12] | (D) |
| 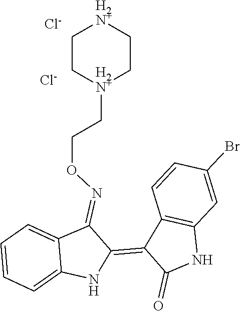 | (E) |
| 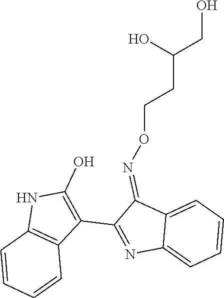 [SMILES: OCC(O)CCO/N=C1/C(c2c(O)[nH]c3ccccc23)=Nc2ccccc21] | (F) |
| 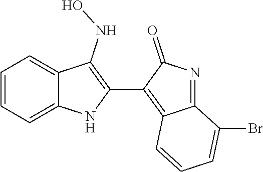 [SMILES: O=C1N=c2c(Br)cccc2=C1c1[nH]c2ccccc2c1NO] | (G) |
| 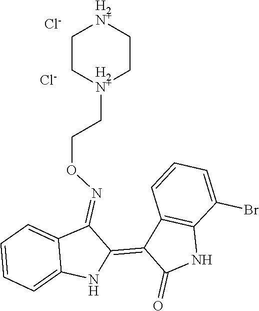 | (H) |
| 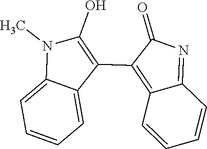 [SMILES: Cn1c(O)c(C2=c3ccccc3=NC2=O)c2ccccc21] | (I) |
| 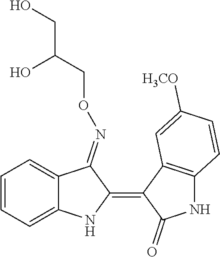 [SMILES: COc1ccc2c(c1)/C(=C1/Nc3ccccc3/C1=N\OCC(O)CO)C(=O)N2] | (J) |
| 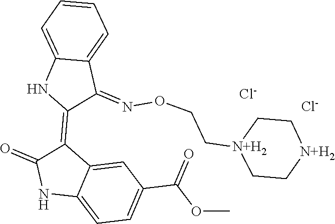 | (K) |
| 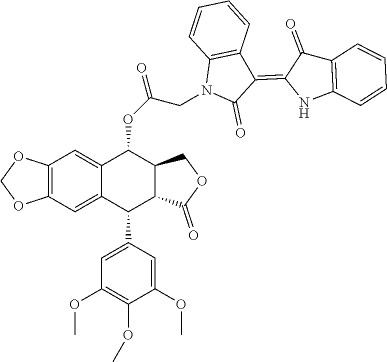 [SMILES: COc1cc([C@@H]2c3cc4c(cc3[C@H](OC(=O)CN3C(=O)/C(=C5\Nc6ccccc6C5=O)c5ccccc53)[C@H]3COC(=O)[C@H]23)OCO4)cc(OC)c1OC] | (L) |
| 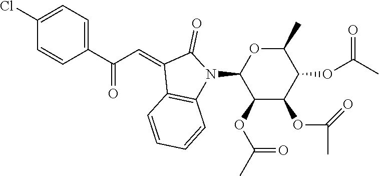 [SMILES: CC(=O)O[C@@H]1[C@@H](OC(C)=O)[C@H](C)O[C@H](N2C(=O)/C(=C/C(=O)c3ccc(Cl)cc3)c3ccccc32)[C@@H]1OC(C)=O] | (M) |
| 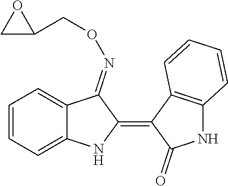 [SMILES: O=C1Nc2ccccc2/C1=C1/Nc2ccccc2/C1=N\OCC1CO1] | (N) |
| 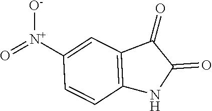 [SMILES: O=C1Nc2ccc([N+](=O)[O-])cc2C1=O] | (O) |
| 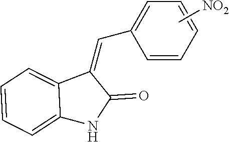 | (P) |

### Role of Indirubin and Derivatives in Cancers

Encouraging clinical results were obtained in the 1980s in chronic myelocytic leukemia (CML) patients treated with indirubin. This stimulated numerous studies on this compound. These investigations explored the use of indirubin in different types of cancer and reported the synthesis of novel derivatives with improved chemical and pharmacokinetic properties. Progress that has been made in elucidating the mechanistic understanding of how indirubin and its derivatives affect physiological and pathophysiological processes, mainly by inhibition of cell proliferation and induction of cell death.²⁴

Although indirubin and IRDs have been shown to have anti-cancer properties (e.g., inhibition of tumor growth, reduction of tumor volume, induction of apoptosis) for some cancers (e.g., CML, adenocarcinoma, prostate cancer, oral cancer), neither indirubin nor IRDs have been shown to be effective against brain cancers.

### Treatment of Brain Cancers

As indicated above, brain cancers such as glioblastoma are considered some of the most challenging of tumors, with little improvement in patient survival in decades. Drug uptake into the brain is generally highly restricted and is considered one of the major barriers in the effective treatment of brain cancer. Treatment of brain tumors with chemotherapy is limited mostly because of delivery impediments related to the blood-brain barrier (BBB). The BBB is composed of microvascular brain endothelial cells, pericytes and astrocytic end-feet and maintains CNS homeostasis by regulating the passage of substances into the brain parenchyma. The BBB represents a formidable challenge for therapeutics to reach the brain parenchyma, which dramatically impacts our capacity to treat CNS-related diseases, such as brain tumors. For gliomas, the most common and aggressive primary brain tumor, treatment includes surgery, radiotherapy, and chemotherapy usually administered orally or intravenously. These routes do not deliver effective concentrations. To complicate matters, chemotherapy is usually a long treatment. Therefore, transient disruption of the BBB is likely insufficient to deliver effective intratumoral concentrations of anticancer drugs. There has been very little to improve outcomes for these patients in decades, thus a new therapy would be rapidly and broadly adopted.

The blood-tumor barrier (BTB) is an interface between brain tumors and blood vessels, and also prevents drug entry. In brain cancers, the integrity of the BTB is heterogeneous, causing unequal drug distribution across the tumor and reducing effective concentrations required to achieve a clinical response. The present invention is based on the observation that indirubin and IRDs enhances intratumoral uptake of anticancer therapeutics in gliomas by disrupting the BTB thereby allowing increased intratumoral anticancer drug accumulation. See, Examples below. Since studies have shown that increasing drug accumulation intratumorally leads to improved outcomes,^{28,30} the combination of indirubin or an IRD as a permeabilizing agent with one or more anticancer drug will provide an effective therapeutic approach to the treatment of brain cancer.

The embodiments of the present invention provide a method for treating a subject afflicted with a malignant brain tumor comprising administering to the subject: (i) a chemotherapeutic agent; and (ii) indirubin or a derivative thereof. The indirubin or a derivative thereof enhances the uptake of the chemotherapeutic agent into the brain tumor. In some embodiments, the indirubin derivative is 6-bromo-indirubin acetoxime (BiA). In some embodiments, the chemotherapeutic agent is selected from carboplatin, carmustine, cisplatin, cyclophosphamide, etoposide, irinotecan, lomustine, methotrexate, procarbazine, temozolomide, and/or vincristine.

In some embodiments, the chemotherapeutic agent is selected from a platelet-derived growth factor receptor (PDGFR) inhibitor, a vascular endothelial growth factor (VEGF) inhibitor, a broad-selectivity kinase inhibitor, a PI3K inhibitor, a GSK3 inhibitor, a Src inhibitor, and/or a Janus kinase inhibitor (JAK inhibitor). In some embodiments, the inhibitor of PDGFR is selected from Imatinib, Axitinib, Cediranib, Foretinib, Imatinib mesylate, Linifanib, Masitinib, Nintedanib, Ponatinib, Sorafenib, Sunitinib malate, AC 710, AP 24534, CP 673451, DMPQ dihydrochloride, JNJ 10198409, KG 5, PD 166285 dihydrochloride, SU 16f, and/or SU 6668. In some embodiments, the inhibitor of VEGFR is selected from Votrient (pazopanib), Sutent (sunitinib), Avastin (bevacizumab), Nexavar (sorafenib), Stivarga (regorafenib), Cabometyx (cabozantinib), Lenvima (lenvatinib), Iclusig (ponatinib), Cometriq (cabozantinib), Zaltrap (ziv-aflibercept), Inlyta (axitinib), Zirabev (bevacizumab), Vegzelma (bevacizumab), Mvasi (bevacizumab), Fotivda (tivozanib), Cyramza (ramucirumab), Caprelsa (vandetanib), and/or Alymsys (bevacizumab). In some embodiments, the broad-selectivity kinase inhibitor is selected from bosutinib, crizotinib, dasatinib, erlotinib, gefitinib, lapatinib, pazopanib, ruxolitinib, sunitinib, and vemurafenib. In some embodiments, the PI3K inhibitor is selected from Buparlisib (BKM120), Zydelig (idelalisib), Piqray (alpelisib), Vijoice (alpelisib), Copiktra (duvelisib), and/or Aliqopa (copanlisib). In some embodiments, the inhibitor of GSK3 inhibitor is selected from elraglusib, Indirubin-3′-oxime (IDR30, 130), (E/Z)-GSK-3p inhibitor 1,7-bromoindirubin-3-oxime (7BIO), WAY-119064, PF-04802367 (PF-367), RGB-286638 free base, Paeoniae Radix Rubra Extract, Alsterpaullone (Alp, 9-Nitropaullone, NSC 705701), SB216763, Laduviglusib (CHIR-99021, CT99021), AT7519, TWS119, Indirubin (NSC 105327), SB415286, CHIR-98014 (CT98014), Tideglusib (NP031112, NP-12), Laduviglusib (CHIR-99021; CT99021), TDZD-8 (NP 01139), Resibufogenin (Bufogenin, Recibufogenin), 5-Bromoindole, LY2090314, AZD1080, 1-Azakenpaullone (1-Akp), BIO (GSK-3 Inhibitor IX, 6-bromoindirubin-3-oxime, 6-Bromoindirubin-3′-oxime, MLS 2052), AZD2858, AR-A014418, IM-12, Bikinin, BIO-acetoxime, CP21R7 (CP21), 9-ING-41, BRD0705, MAZ51, and/or Chonglou Saponin VII. In some embodiments, the inhibitor of Src is selected from Dasatinib, Alsterpaullone, Bosutinib, Herbimycin A, Piceatannol, Saracatinib, Squarunkin A hydrochloride, Tilfrinib, A 419259 trihydrochloride, AZM 475271, JNJ 10198409, KB SRC 4, KX2-391, LCB 03-0110 dihydrochloride, PD 166285 dihydrochloride, PKI 166 hydrochloride, PP1, 1-Naphthyl PP1, PP2, PP3, Src 11, SU 6656, TC-S 7003, TL 0259, and/or WH-4-023. In some embodiments, the JAK inhibitor is selected from Jakafi (ruxolitinib), Cibinqo (abrocitinib), Inrebic (fedratinib), Olumiant (baricitinib), Opzelura (ruxolitinib), Rinvoq (upadacitinib), and/or Xeljanz (tofacitinib).

In some embodiments, the technology provides a method for treating a subject afflicted with a malignant brain tumor, the method comprising administering to the subject: (i) a chemotherapeutic agent; and (ii) indirubin or a derivative thereof, wherein the indirubin or a derivative thereof enhances the uptake of the chemotherapeutic agent into brain tumors. According to some aspects, the method is executed wherein the indirubin or a derivative thereof includes a compound of formula 1:

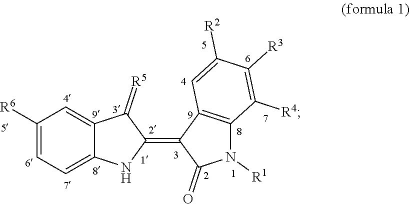

wherein R¹ is selected from —H, —CH₃, and —CH₂CH₃;
wherein R² is selected from —H, —SO₃H, —SO₂NH₂, —OCH₃, —NHCO-tert-Butyl, —NO₂, —NHCOCH(C₆H₅)₂, —NO₂, —Br, —I, —Cl, -and —F;
wherein R³ and R⁴ are each independently selected from —H, —Br, —I, —Cl, -and —F;
wherein R⁵ is selected from ═O, =NOH, =NOCH₃, =NOCOCH₃, =NOCH₂CH₂OCH₂CH₂, and =NOCH₂CH₂CH(OH)CH₂OH.
wherein R⁶ is selected from —H and —COOH;
or a pharmaceutically acceptable salt thereof.

In some embodiments, the method above is further comprising that the method can be executed as a method for enhancing the uptake of a chemotherapeutic agent into a brain tumor in a subject in need thereof, the method comprising administering to the subject (i) a chemotherapeutic agent; and (ii) indirubin or a derivative thereof as described above.

According to some aspects, a pharmaceutically acceptable salt described above includes an anion comprising bromide, carbide, chloride, fluoride, hydride, iodide, nitride, phosphide, oxide, sulfide, selenide, azide, peroxide, triodide, carbonate, chlorate, chromate, dichromate, dihydrogen phosphate, hydrogen carbonate, hydrogen sulfate, hydrogen sulfite, hydroxide, hypochlorite, monohydrogen phosphate, nitrate, nitrite, perchlorate, permanganate, peroxide, phosphate, sulfate, sulfite, superoxide, thiosulfate, silicate, metasilicate, aluminium silicate, acetate, formate, or oxalate.

In some embodiments, the indirubin derivative is 6-bromo-indirubin acetoxime (bia). According to some aspects, the indirubin derivative can be selected from Table 3.

According to some aspects, the indirubin derivative is selected from the indirubin derivatives shown in Table 1 or Table 2.

In some embodiments, the method can be wherein the chemotherapeutic agent is selected from the group consisting of: carboplatin, carmustine, cisplatin, cyclophosphamide, etoposide, irinotecan, lomustine, methotrexate, procarbazine, temozolomide, and vincristine.

According to some aspects, the method is executed wherein the chemotherapeutic agent is selected from the group consisting of: a platelet-derived growth factor receptor (PDGFR) inhibitor, a vascular endothelial growth factor (VEGF) inhibitor, a broad-selectivity kinase inhibitor, a PI3K inhibitor, a GSK3 inhibitor, a Src inhibitor, and a Janus kinase inhibitor (JAK inhibitor). In some embodiments, the PDGFR inhibitor is selected from the group consisting of: Imatinib, Axitinib, Cediranib, Foretinib, Imatinib mesylate, Linifanib, Masitinib, Nintedanib, Ponatinib, Sorafenib, Sunitinib malate, AC 710, AP 24534, CP 673451, DMPQ dihydrochloride, JNJ 10198409, KG 5, PD 166285 dihydrochloride, SU 16f, and SU 6668.

According to some aspects, the VEGF inhibitor is selected from the group consisting of: Votrient (pazopanib), Sutent (sunitinib), Avastin (bevacizumab), Nexavar (sorafenib), Stivarga (regorafenib), Cabometyx (cabozantinib), Lenvima (lenvatinib), Iclusig (ponatinib), Cometriq (cabozantinib), Zaltrap (ziv-aflibercept), Inlyta (axitinib), Zirabev (bevacizumab), Vegzelma (bevacizumab), Mvasi (bevacizumab), Fotivda (tivozanib), Cyramza (ramucirumab), Caprelsa (vandetanib), and Alymsys (bevacizumab).

In some embodiments, the broad-selectivity kinase inhibitor can be selected from the group consisting of: bosutinib, crizotinib, dasatinib, erlotinib, gefitinib, lapatinib, pazopanib, ruxolitinib, sunitinib, and vemurafenib.

According to some aspects, the PI3K inhibitor is selected from the group consisting of: Buparlisib (BKM120), Zydelig (idelalisib), Piqray (alpelisib), Vijoice (alpelisib), Copiktra (duvelisib), and Aliqopa (copanlisib).

In some embodiments, the GSK3 inhibitor is selected from the group consisting of: elraglusib, Indirubin-3′-oxime (IDR30, 130), (E/Z)-GSK-3p inhibitor 1, 7-bromoindirubin-3-oxime (7BIO), WAY-119064, PF-04802367 (PF-367), RGB-286638 free base, Paeoniae Radix Rubra Extract, Alsterpaullone (Alp, 9-Nitropaullone, NSC 705701), SB216763, Laduviglusib (CHIR-99021, CT99021), AT7519, TWS119, Indirubin (NSC 105327), SB415286, CHIR-98014 (CT98014), Tideglusib (NP031112, NP-12), Laduviglusib (CHIR-99021; CT99021), TDZD-8 (NP 01139), Resibufogenin (Bufogenin, Recibufogenin), 5-Bromoindole, LY2090314, AZD1080, 1-Azakenpaullone (1-Akp), BIO (GSK-3 Inhibitor IX, 6-bromoindirubin-3-oxime, 6-Bromoindirubin-3′-oxime, MLS 2052), AZD2858, AR-A014418, IM-12, Bikinin, BIO-acetoxime, CP21R7 (CP21), 9-ING-41, BRD0705, MAZ51, and Chonglou Saponin VII.

According to some aspects, the Src inhibitor is selected from the group consisting of: Dasatinib, Alsterpaullone, Bosutinib, Herbimycin A, Piceatannol, Saracatinib, Squarunkin A hydrochloride, Tilfrinib, A 419259 trihydrochloride, AZM 475271, JNJ 10198409, KB SRC 4, KX2-391, LCB 03-0110 dihydrochloride, PD 166285 dihydrochloride, PKI 166 hydrochloride, PP1, 1-Naphthyl PP1, PP2, PP3, Src 11, SU 6656, TC-S 7003, TL 0259, and WH-4-023.

In some embodiments, the JAK inhibitor is selected from the group consisting of: Jakafi (ruxolitinib), Cibinqo (abrocitinib), Inrebic (fedratinib), Olumiant (baricitinib), Opzelura (ruxolitinib), Rinvoq (upadacitinib), and Xeljanz (tofacitinib).

According to some aspects, the administration of the chemotherapeutic agent is done concomitantly or sequentially with the administration of indirubin or a derivative thereof.

In some embodiments, the administration of the chemotherapeutic agent and indirubin or a derivative thereof is done concomitantly or sequentially with one or more of a surgical resection, radiotherapy, antiangiogenic therapy, immune therapy, gamma knife radiosurgery, and symptomatic management with corticosteroids.

According to some aspects, the method described above is wherein the malignant brain tumor is selected from the group consisting of: astrocytoma, glioblastoma, oligodendrocytoma, pilocytic astrocytoma, diffuse intrinsic pontine glioma, ependymoma, oligo-astrocytoma, oligodendrogliocytoma, optic pathway glioma, and hypothalamic glioma.

### Modulation of Blood-Tumor Barrier Transcriptional Programs Improves Intratumoral Drug Delivery and Potentiates Chemotherapy in Murine GBM

Glioblastoma (GBM) is the most common malignant primary brain tumor. Only 5% of patients survive five years post diagnosis. In addition to extensive intra-tumoral heterogeneity found in GBM, the blood-tumor barrier represents a major challenge to medically manage this disease. The blood-tumor barrier prevents therapeutic drugs from reaching appropriate intra-tumoral doses, dramatically hindering their potential. Here, we identified a set of 12 genes associated to blood-tumor barrier functions, revealing vasculature development, cell migration and morphogenesis processes to be highly active in tumoral vasculature regions. We identified CDH5 as a core molecule in this set and confirmed its over-expression in GBM patient samples using spatial transcriptomics. Using an indirubin-derivative, 6-bromoindirubin acetoxime (BIA), we modulated the expression of CDH5 and other brain-tumor barrier-associated genes, causing endothelial barrier disruption in endothelial monolayers and blood-brain barrier (BBB) 3D spheroids in vitro. This property of BIA allowed for increased chemotherapy intra-tumoral accumulation in vivo and potentiated the anti-neoplastic capacity of cisplatin by targeting DNA repair pathways. Finally, using an injectable BIA formulation, PPRX-1701, we improved cisplatin effects in patient-derived GBM xenografts and prolonged their survival significantly. Overall, our work reveals potential targets of the blood-tumor barrier for improved chemotherapy delivery and the bifunctional properties of BIA as a tumor vasculature permeability modulator and potentiator of chemotherapy, prompting its applicability in further pre-clinical and clinical settings.

Introduction: The effective treatment of brain malignancies such as glioblastoma (GBM), remains a critical challenge in the neuro-oncology field. GBM is the most common malignant primary brain tumor, representing ˜15% of all central nervous system (CNS) neoplasms.³¹ Median survival is 15-18 months and only 5% of patients survive beyond 5 years after diagnosis.³² The standard of care involves maximal-safe surgical resection, followed by radiotherapy and temozolomide (TMZ) inevitably leads to the development of untreatable recurrent disease. The major challenges in GBM therapy are 1) its invasiveness, which prevents complete surgical resection, 2) high levels of intra-tumoral molecular and cellular heterogeneity, 3) a cancer-promoting tumor microenvironment (TME), and 4) the presence of the blood-brain barrier (BBB) and blood-tumor barrier (BTB), that limit drug entry.

The BBB maintains homeostasis of the central nervous system (CNS) for proper functioning.³³ Under an oncogenic context, the BBB responds to cues by the cancer cells, which promote the formation of a blood-tumor barrier (BTB). The BTB is a distinctive biological entity, resulting from cellular interactions between the BBB and brain-tumor cells.³⁴ Molecular characteristics that define the impermeability of the BBB, such as tight junction and adherens junction formations, high efflux pump expression and non-fenestrated endothelium, are compromised in brain tumors in turn for hypoxic/angiogenic conditions that promote tumor growth, migration and invasion.³⁵ Regardless of the disruption of these brain-protecting BBB properties, non-BBB penetrant drugs remain inefficient to combat GBM. Several studies suggest that the BTB is highly heterogeneous as well,³⁶ with some regions maintaining “healthy” BBB features that protect GBM cells from antineoplastic agent accumulation.

Many biological features of the BTB remain to be known, such as its molecular origins upon brain tumorigenesis, and identification of target molecular pathways that could render the BTB permissive to chemotherapy tumoral uptake. Some strategies to improve drug delivery to GBM involve focused ultrasound (FUS),³⁷,3⁸ convection-enhanced mediated delivery (CED),³⁹ optogenetics,⁴⁰ systemic administration of drug-loaded nanoparticles,⁴¹ and drug-conjugated cell-penetrating peptides,⁴² with most of these options showing promising pre-clinical results. These strategies rely on physically overcoming the BTB, and present advantages on controlled release, preservation of drug stability and drug delivery at selected anatomical sites. Yet, identification of compounds that could target molecular elements that selectively regulate BTB permeability, but not healthy BBB, would enable mechanistic control over the biological processes involving the BTB/GBM tumor interactions, and could be well used to potentiate intra-tumoral drug penetration. Moreover, if these compounds could simultaneously hinder tumor development and synergize with chemotherapeutic regimens, then improved clinical outcomes should be expected for brain cancer patients.

Indirubins are bisindole alkaloid compounds used in traditional Chinese medicine for the treatment of proliferative disorders and auto-immune conditions. Indirubin is a component from the Indigo naturalis extract derived from plants such as Baphicacanthus cusia, Polygonum tinctorium and Isatis indigotica.²⁷ Indirubins can be chemically modified to modulate their chemical and biological properties, although most of them are recognized as potent multi-kinase inhibitors. The 6-bromoindirubin acetoxime (BIA) derivative, is widely known as a GSK-3 inhibitor,⁴³ but other target kinases have been found to be inhibited by this compound, including cyclin kinases such as CDK5 and PhK.⁴⁴ Previously, we reported the anti-GBM capacity of BIA, which halted tumor migration and growth in vitro and in vivo. BIA exerted blockage of endothelial migration and reduced angiogenesis of intra-cranial murine GBM models.⁴⁵ Treatment with BIA-loaded nanoparticles, PPRX-1701, reduced the immune-suppressive environment of murine GBM by diminishment of IDO1 expression, which in turn promoted CD8+ cytotoxic lymphocyte infiltration and showed improved pre-clinical efficacy.⁴⁶ In addition, PPRX-1701 was well tolerated, not showing signs of peripheral tissue toxicity, making it a potential candidate for escalation towards clinical applications.

Herein, by establishing an in silico-based approach, we identified 12 BTB-related genes in glioblastoma that integrate into vasculature development, cell migration and morphogenesis processes. Within this gene set, we identified CDH5 (VE-cadherin), as a core molecule of the BTB. Use of BIA targeted CDH5 and other BTB-genes involved in TGF-3, WNT and angiogenic pathways in endothelium. BIA increased cisplatin accumulation in tumor tissue, but not in healthy brain, of xenograft and syngeneic murine GBM, and enhanced the cytotoxic capacity of cisplatin. BIA in combination with cisplatin prolonged survival of xenograft GBM models. Together, our work provides evidence of potential candidate targets of the BTB and the use of BIA for improved drug delivery and chemotherapy potentiation in GBM.

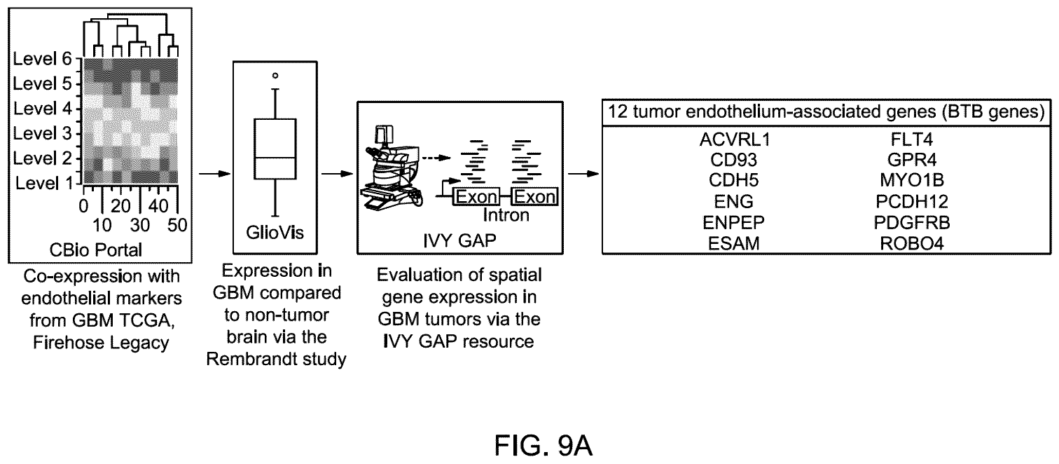

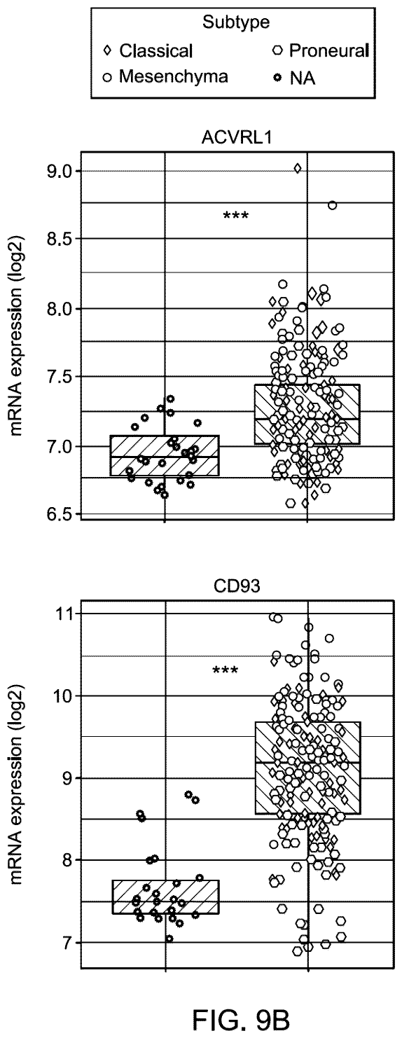

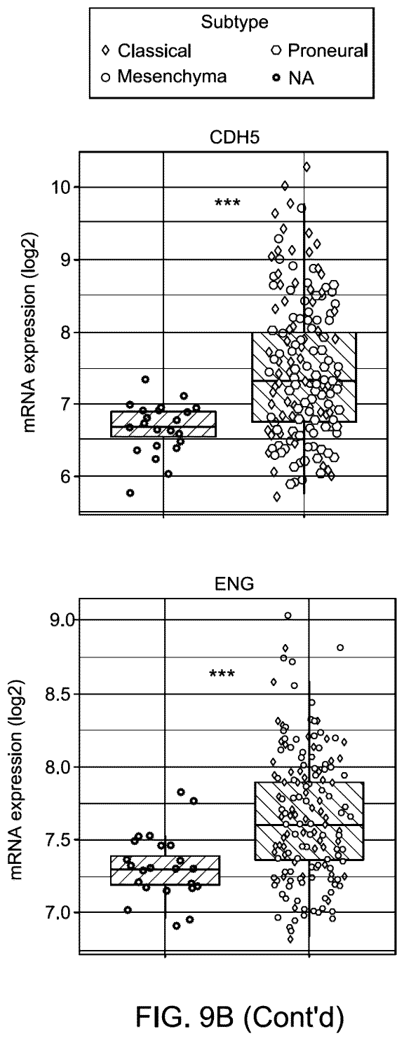

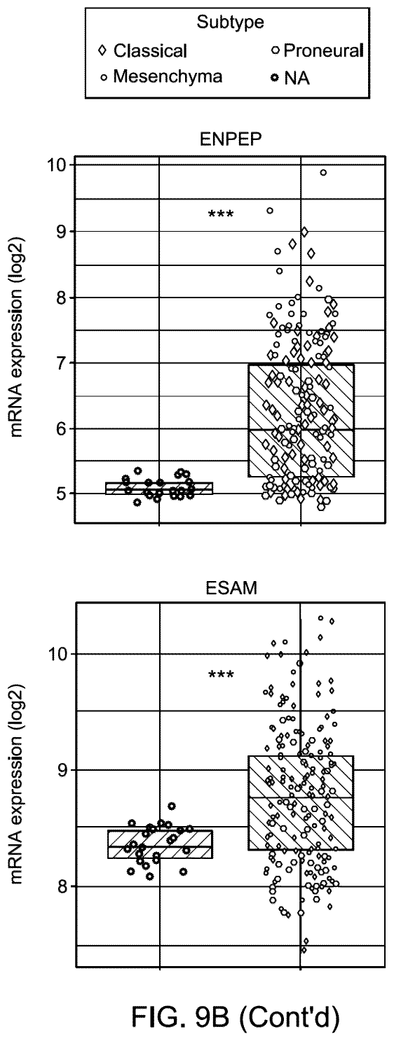

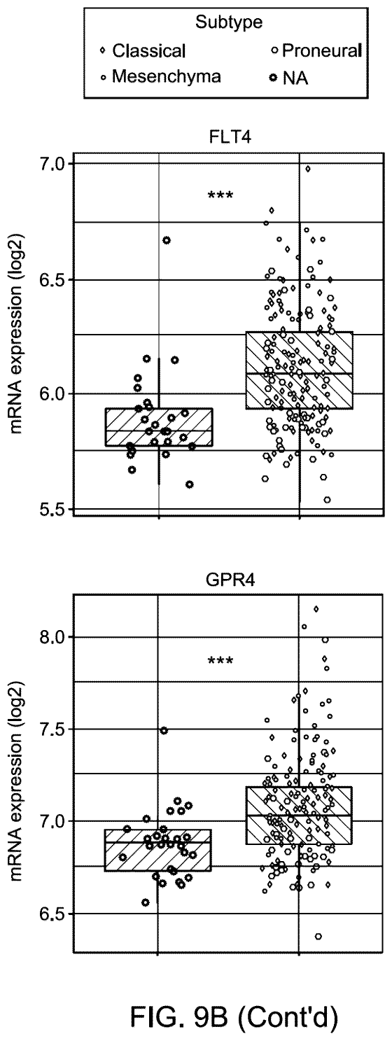

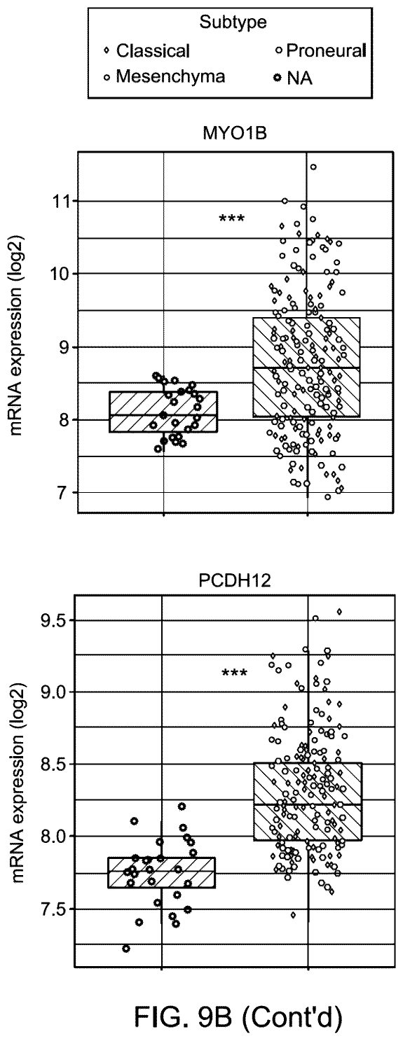

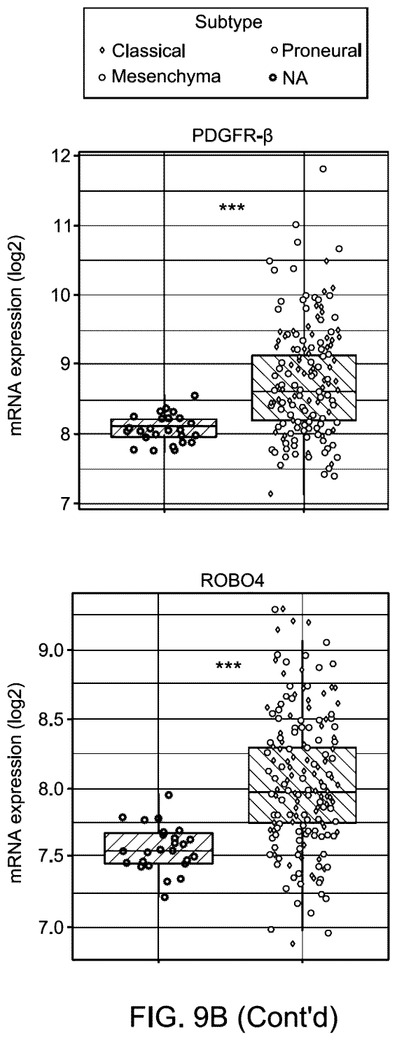

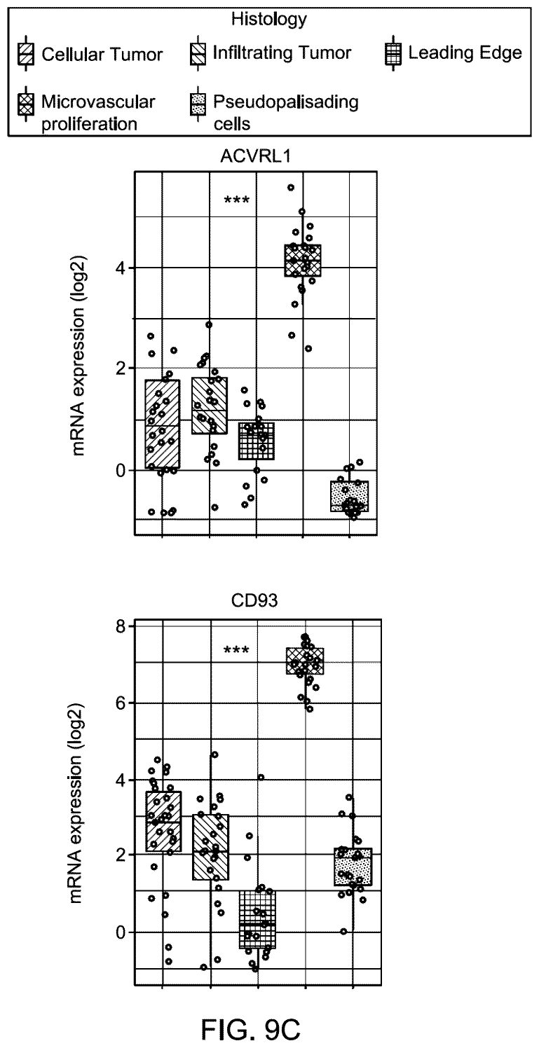

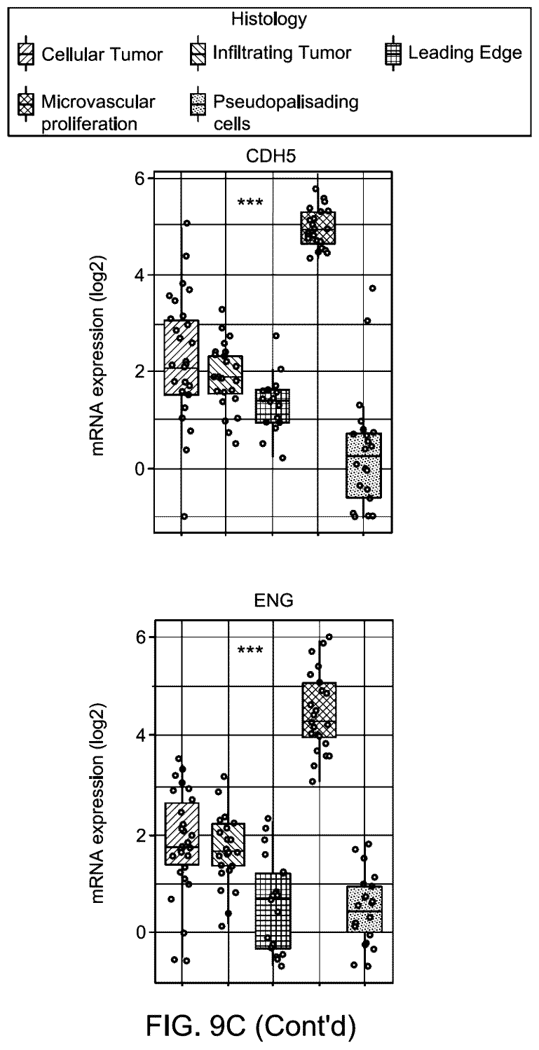

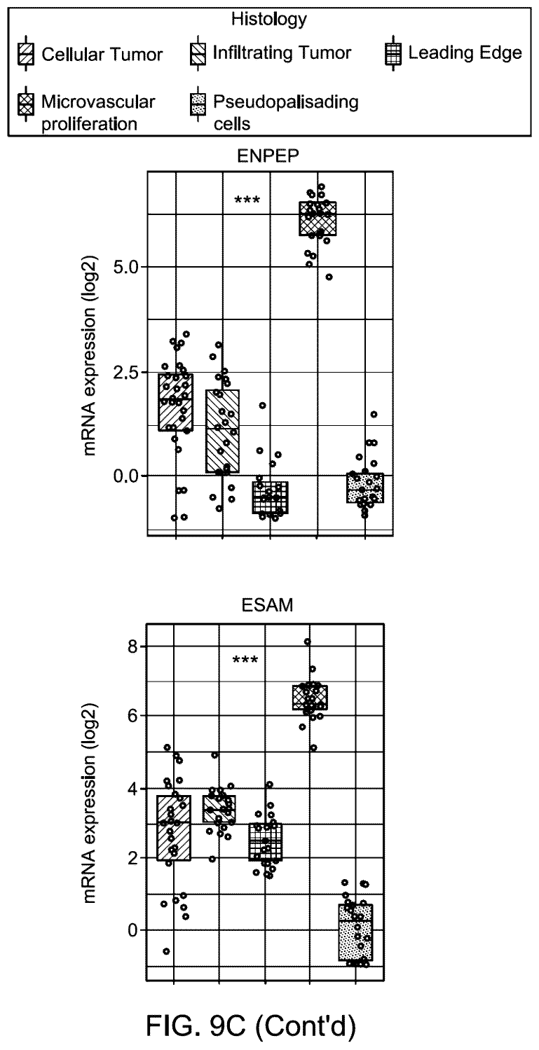

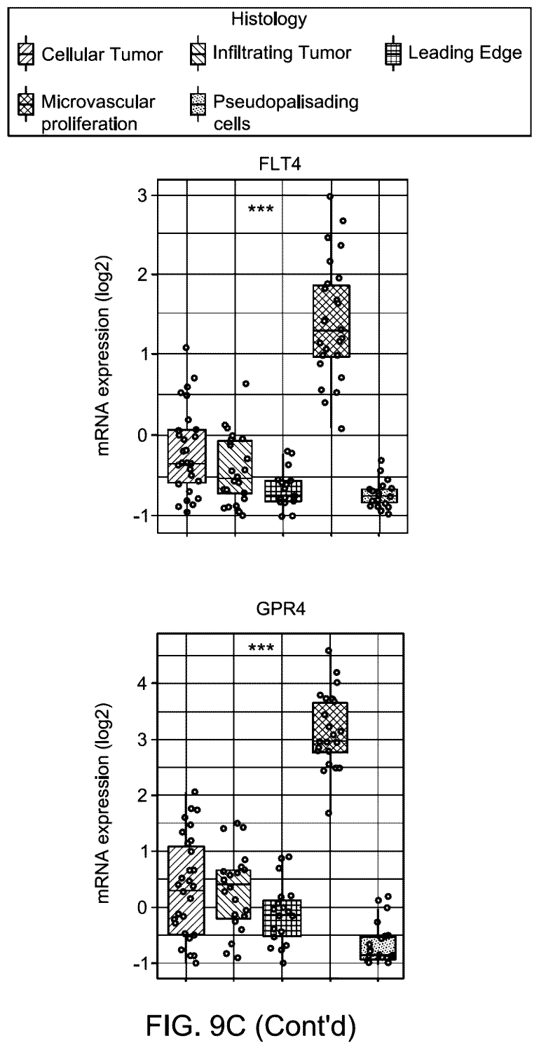

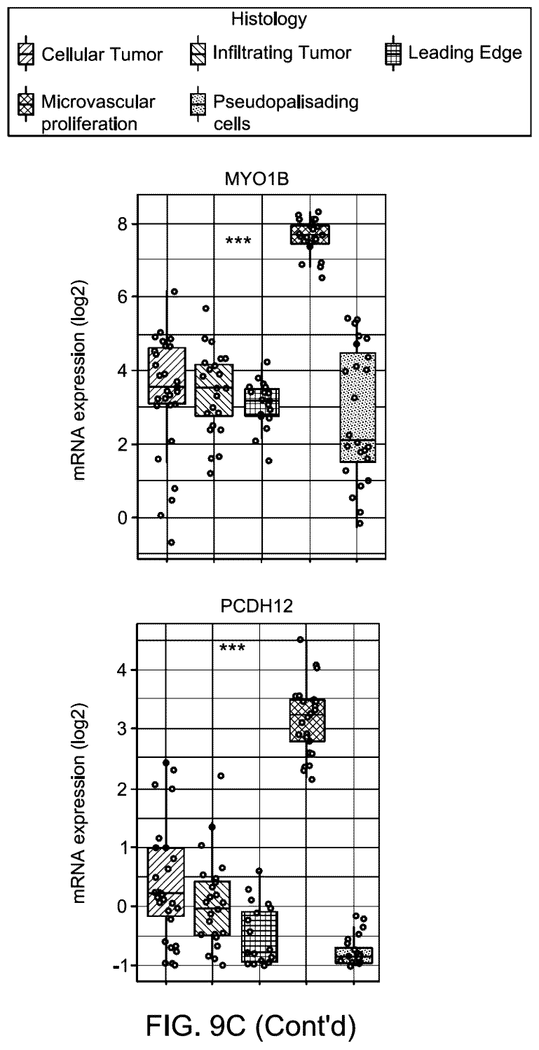

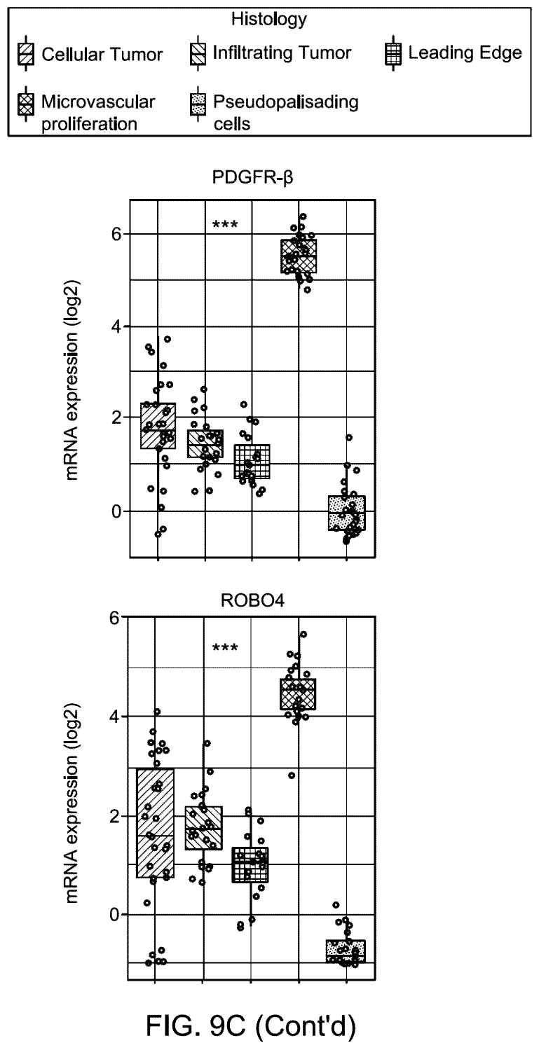

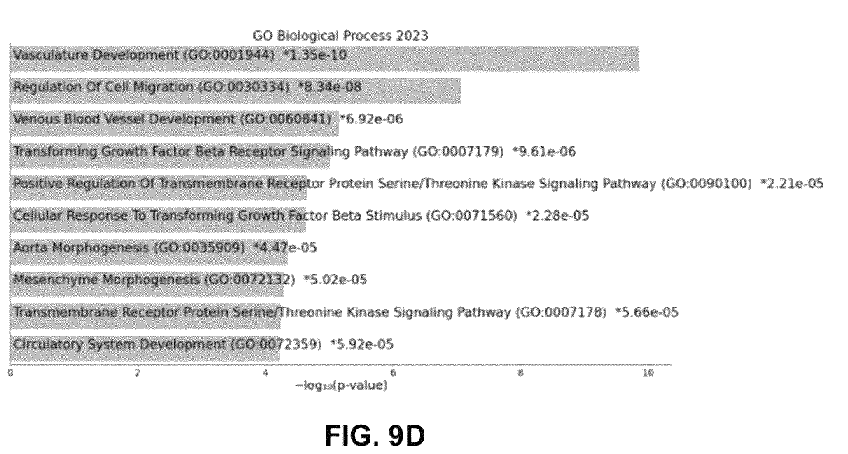

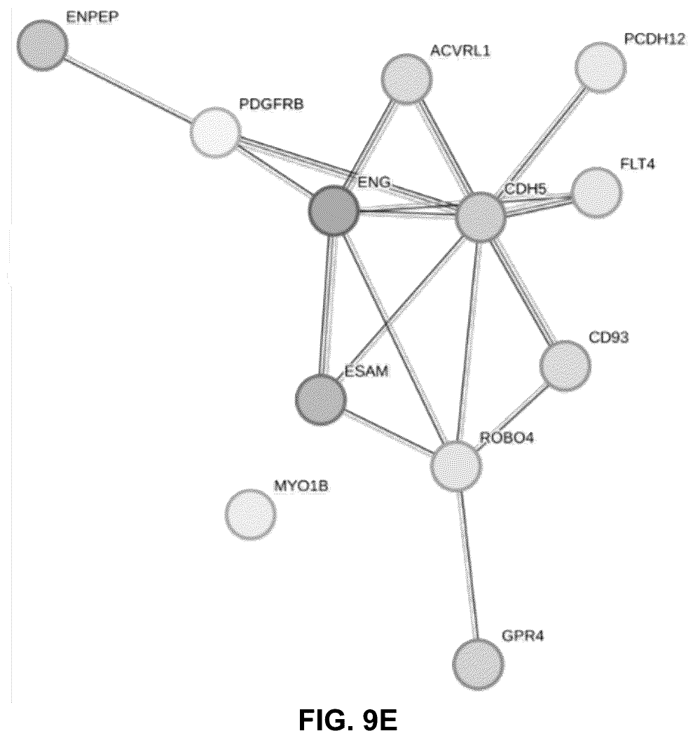

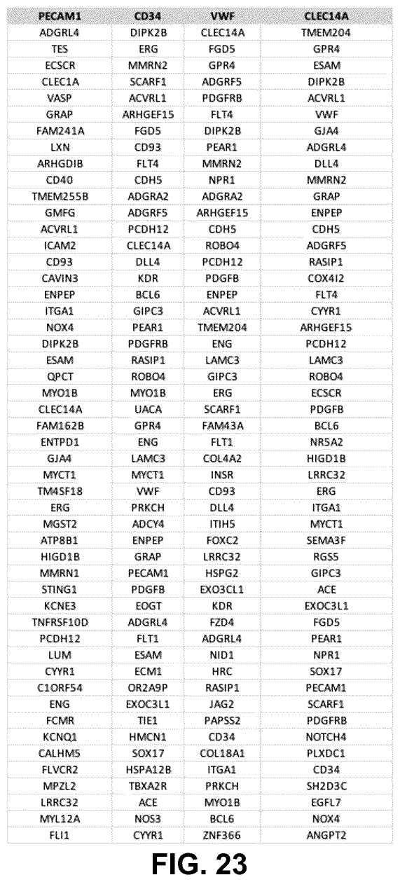

Results, Identification of GBM tumor endothelium-associated genes (BTB-genes) via in silico screening: The BTB represents an obstacle to therapeutic drug delivery and remains a poorly defined component of GBM biology. Thus, to identify molecular signatures of the GBM vasculature for targeting of the BTB, we performed an in silico-based approach by accessing bulk RNA-sequencing data from The Cancer Genome Atlas via the CBIO portal (FIG. 9A). We used Spearman's rank correlation to select GBM genes that showed co-expression with four well-characterized endothelial cell markers defined by Dussart et al (2019)⁴⁷: CD31, CD34, VWF and CLEC14A. (FIG. 23, Supplementary Table 1). With this approach, we identified a signature comprising 12 GBM tumor endothelium-associated genes (BTB-genes) with increased expression in GBM tumors above non-tumoral tissue (FIG. 9B). This included known angiogenesis-associated genes such as ACVRL1 (ALK1), CD93, ENG and PDGFRB, as well as endothelial cell-adhesion endothelial genes PCDH12, ROBO4, ESAM and CDH5. These genes present abundant expression at microvascular proliferation regions in the tumor (FIG. 9C). Gene Ontology analysis revealed their primary involvement in vasculature system development, morphogenesis and cell migration (FIG. 9D). STRING network analysis showed that the genes form significant interaction within the network (PPI enrichment p-value: <1.0e-16). With the exception of MYO1B all genes were interconnected (FIG. 9E), suggesting strong functional relationships within the GBM BTB signature gene set.

Examples of identification of tumor-endothelium associated (BTB-genes) using bulk and spatial RNA-sequencing datasets from GBM clinical samples are shown herein. (FIG. 9A) Workflow of identification and filtering of genes associated with tumoral vasculature in GBM. Using a gene expression correlation tool, cBIO, top-50 genes that co-expressed with CD31, VWF, CD34 and CLEC14A were selected, confirmed their over-expression in tumor above normal brain in the Rembrandt dataset using GlioVis visualization tool, and evaluated regional expression using the IVY GAP atlas resource. (FIG. 9B) Gene expression of 12 tumor endothelium-associated genes (BTB-genes) identified in the cBio Portal following the workflow shown in FIG. 9A, ***p-value<0.001 by Tukey's Honest Significant Difference test. Individual values are colored by GBM subtype Classical, Mesenchymal or Proneural, NA indicates unknown sample information. (FIG. 9C) Regional expression of the 12 BTB-genes in GBM using the IVY GAP resource, ***p-valueti0.001 by Tukey's Honest Significant Difference test. (FIG. 9D) Gene ontology analysis (GO Biological Process 2023) of the 12 BTB-genes using the EnrichR soaware. Biological processes are ranked by p-values, which are indicated next to the GO designa0on. (FIG. 9E) STRING network analysis on the 13 iden0fied BTB-genes. 12 nodes, 17 edges, average node degree 2.83. PPI enrichment p-value 1 e\hat{ }-16.

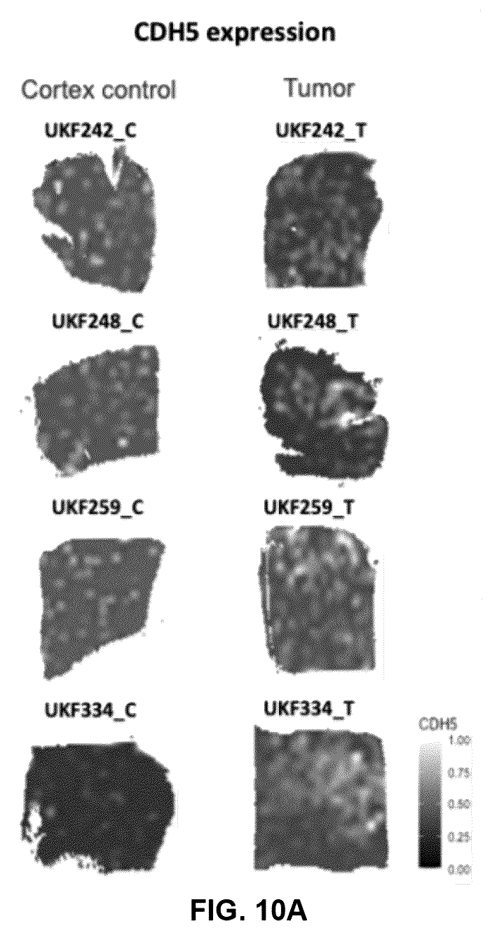

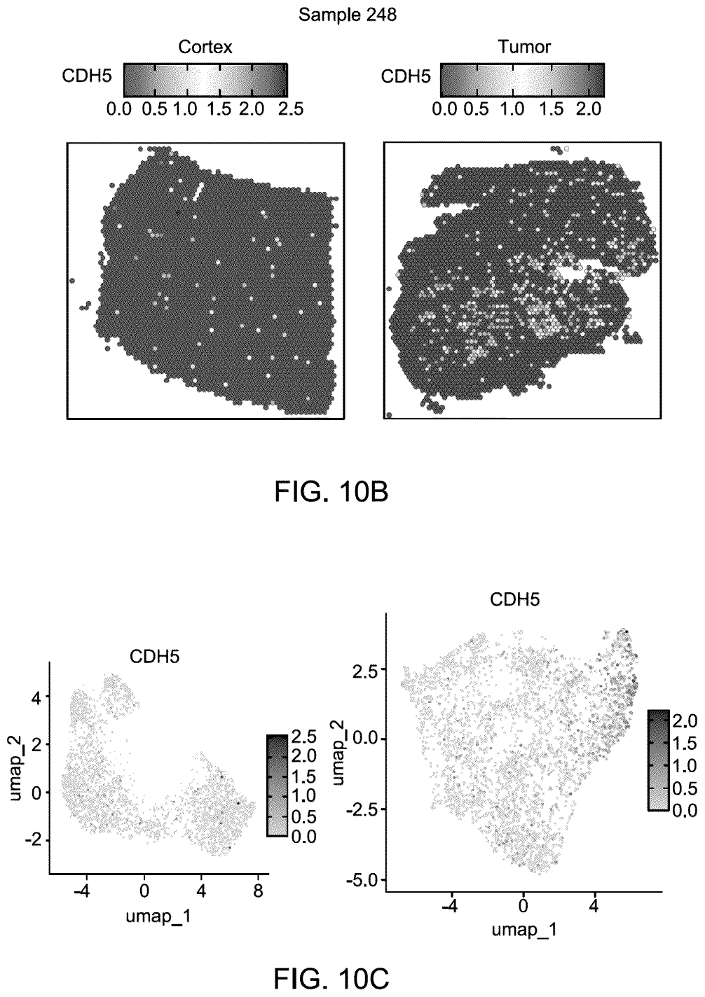

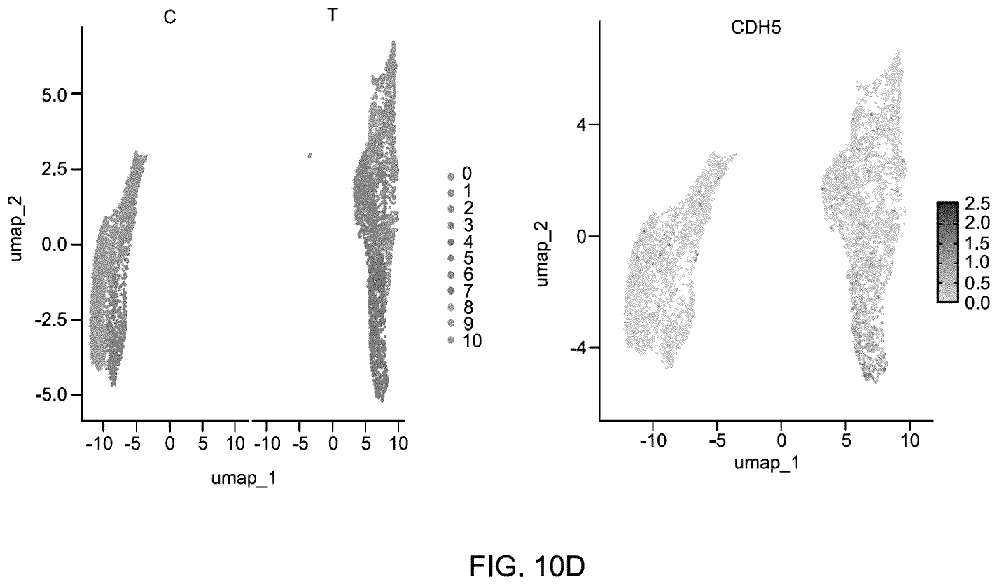

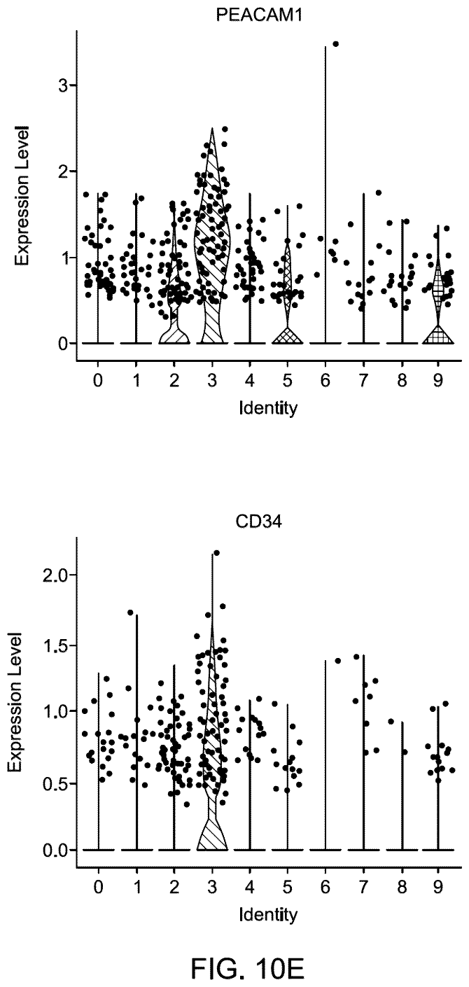

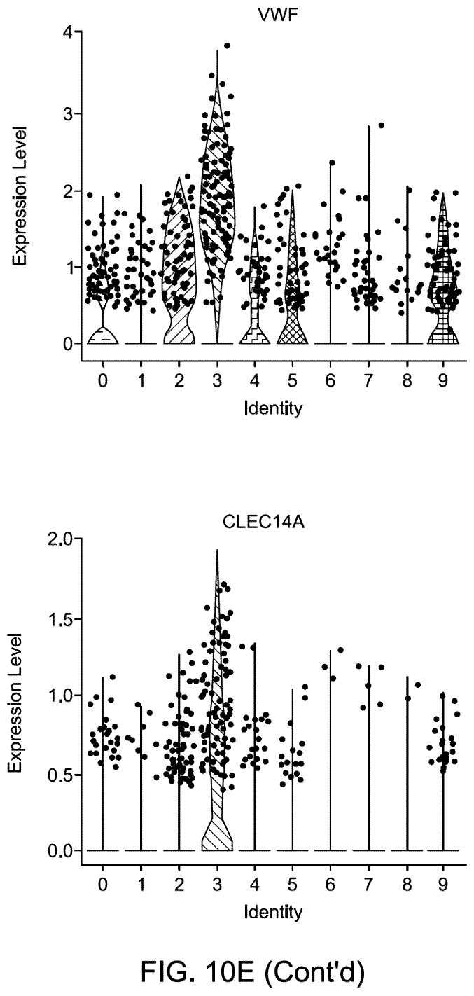

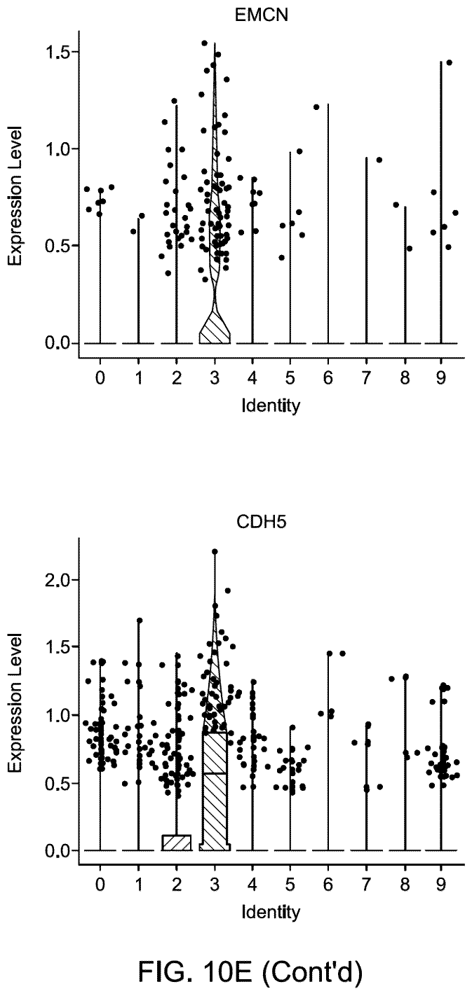

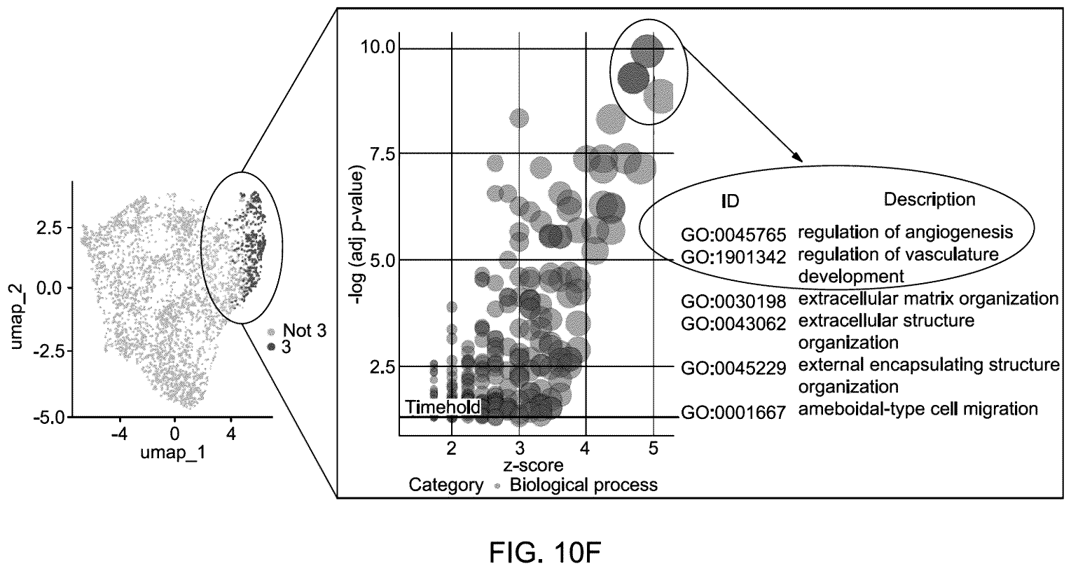

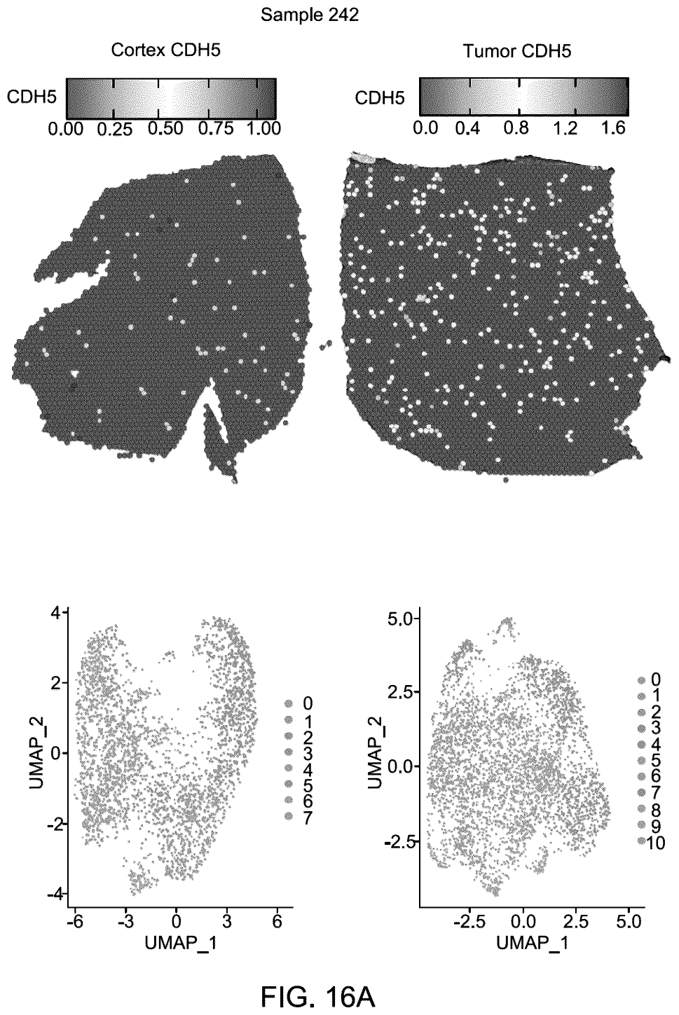

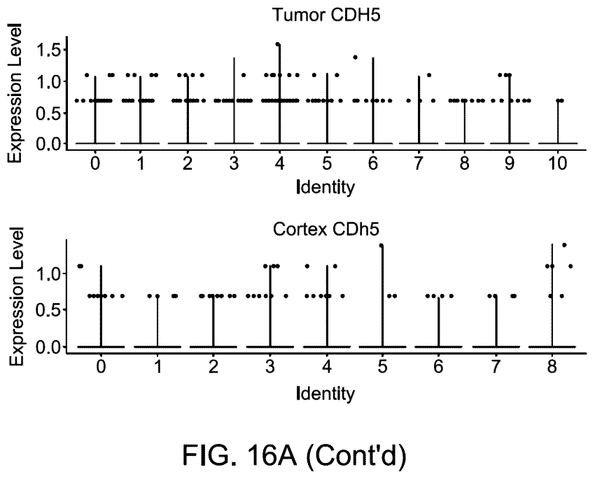

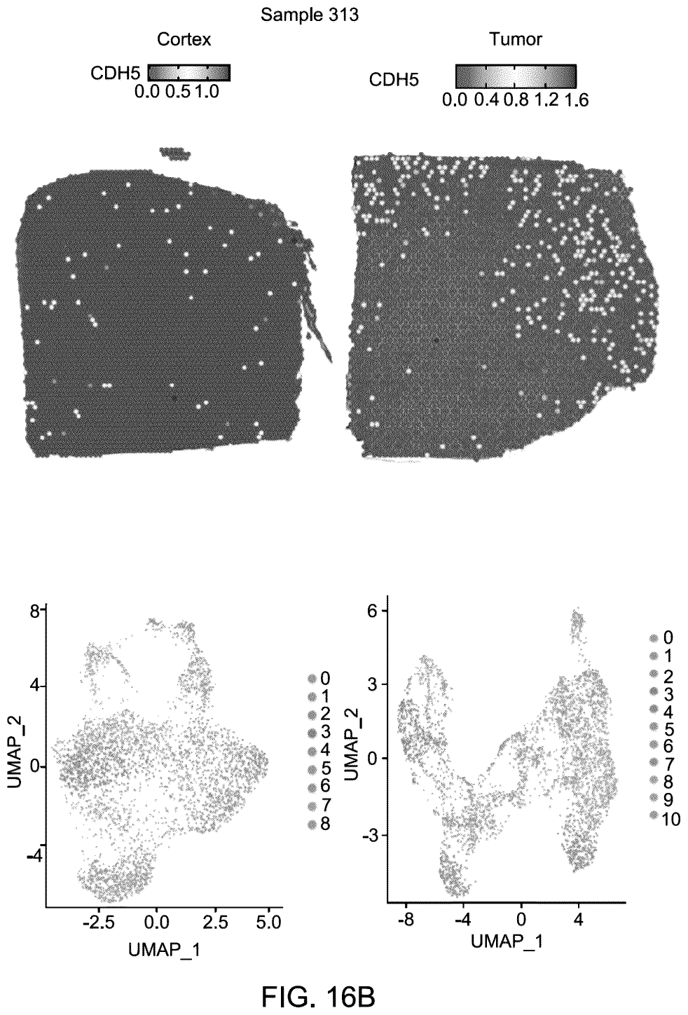

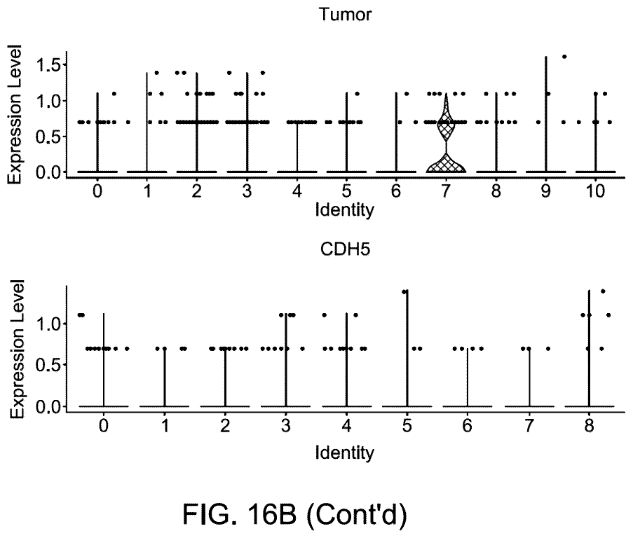

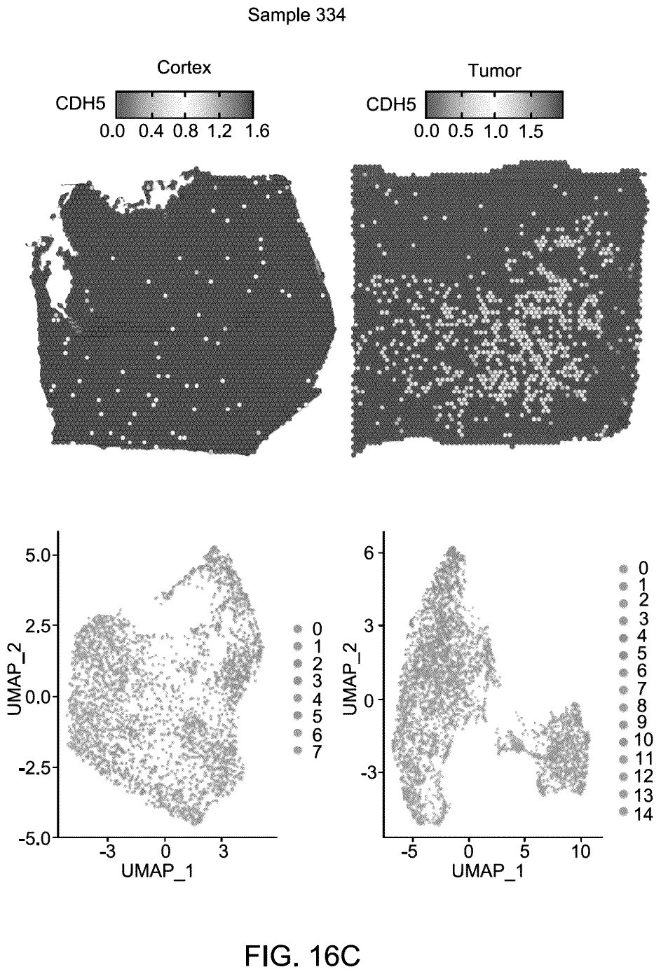

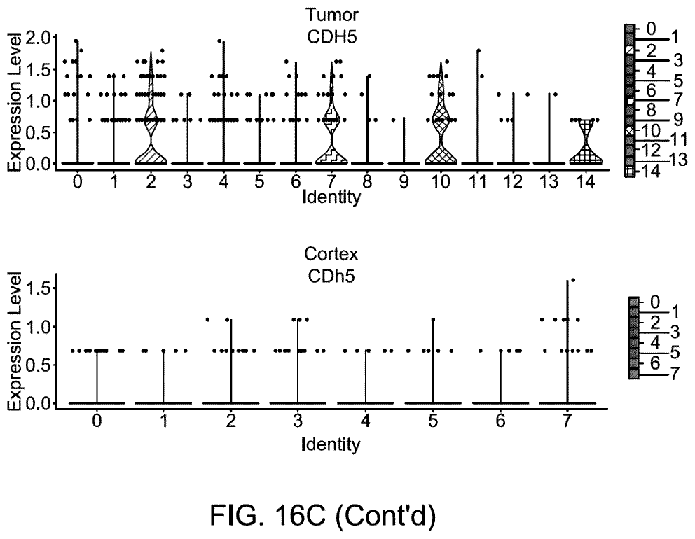

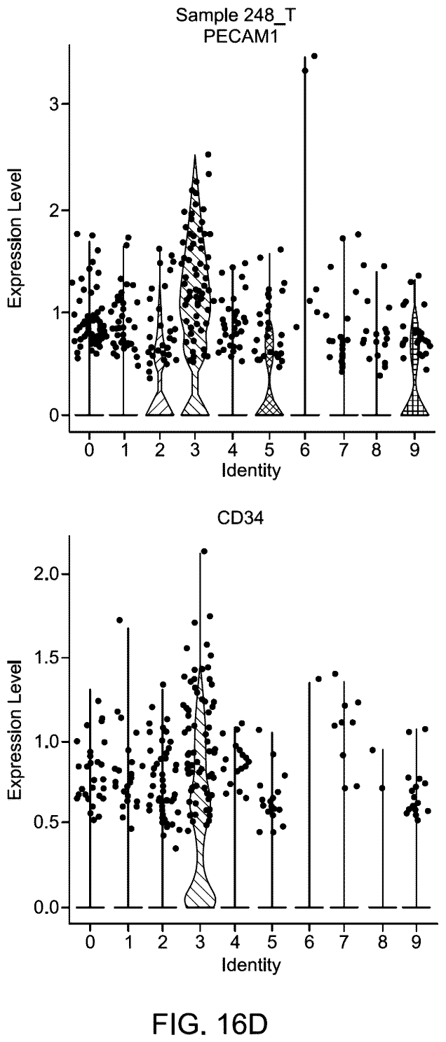

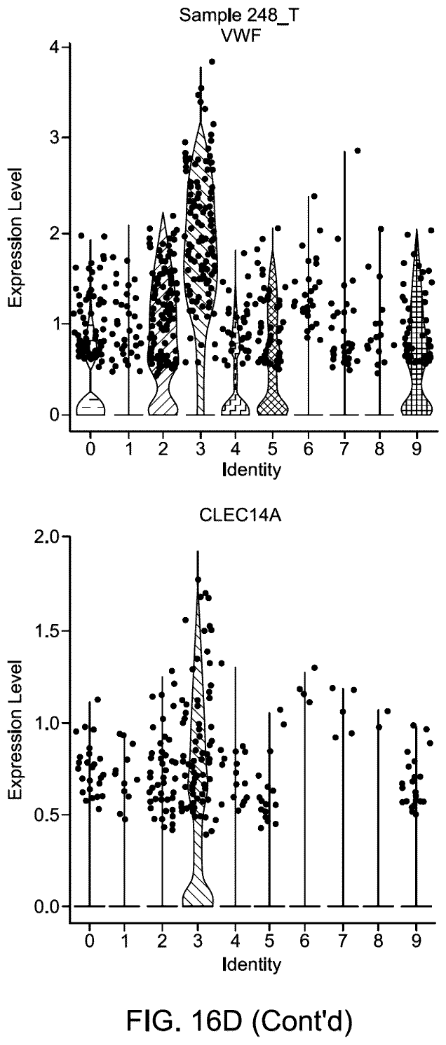

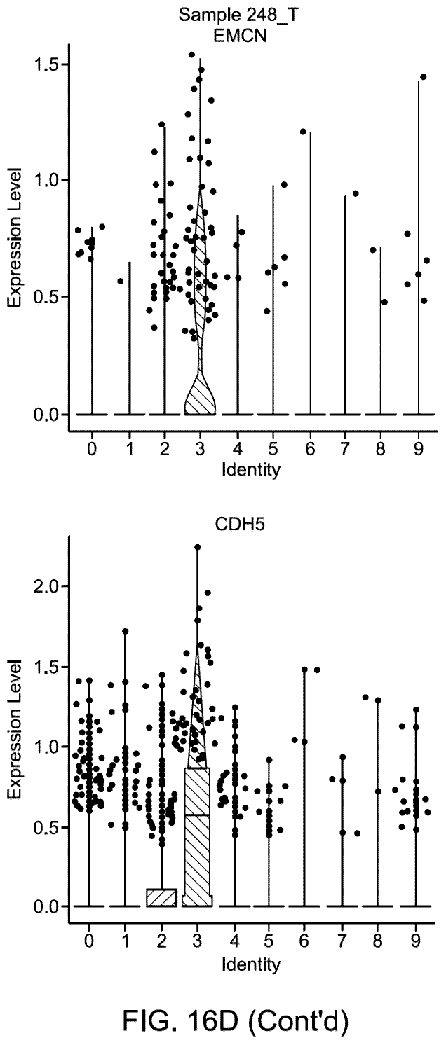

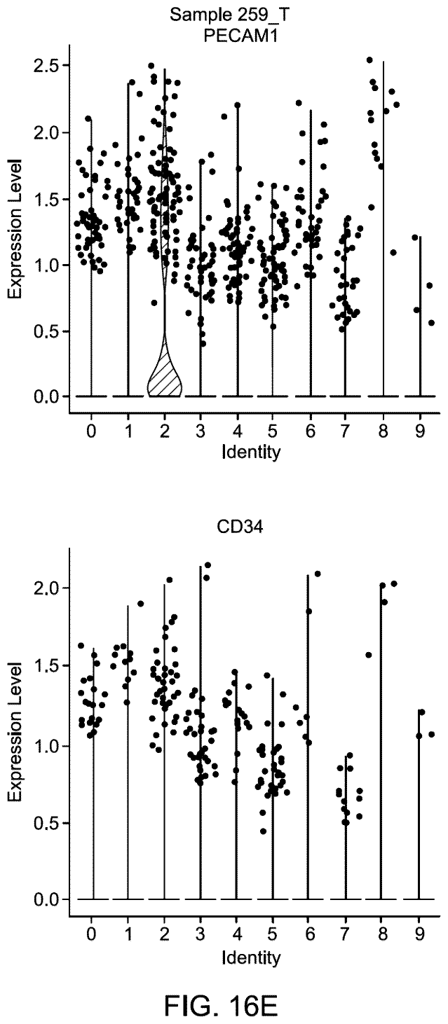

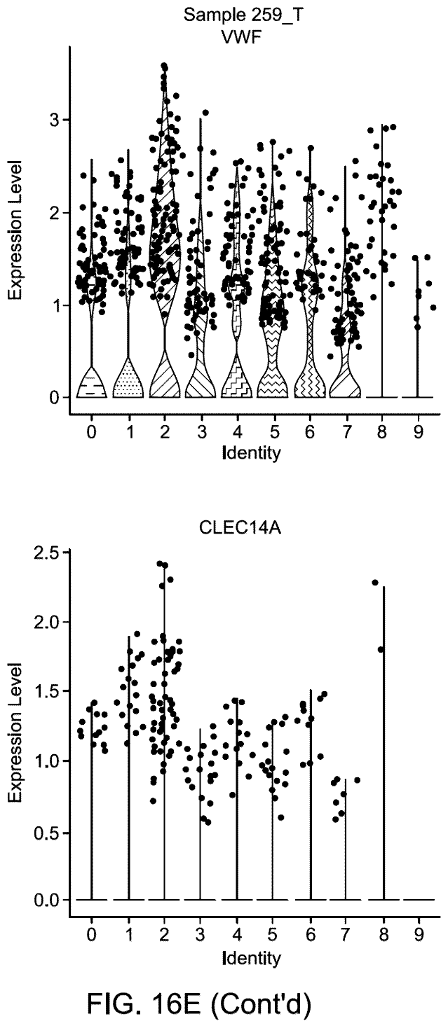

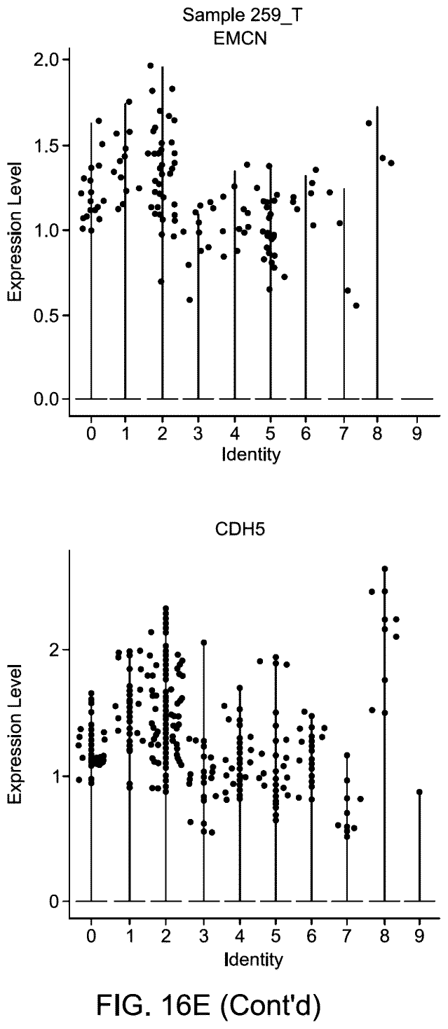

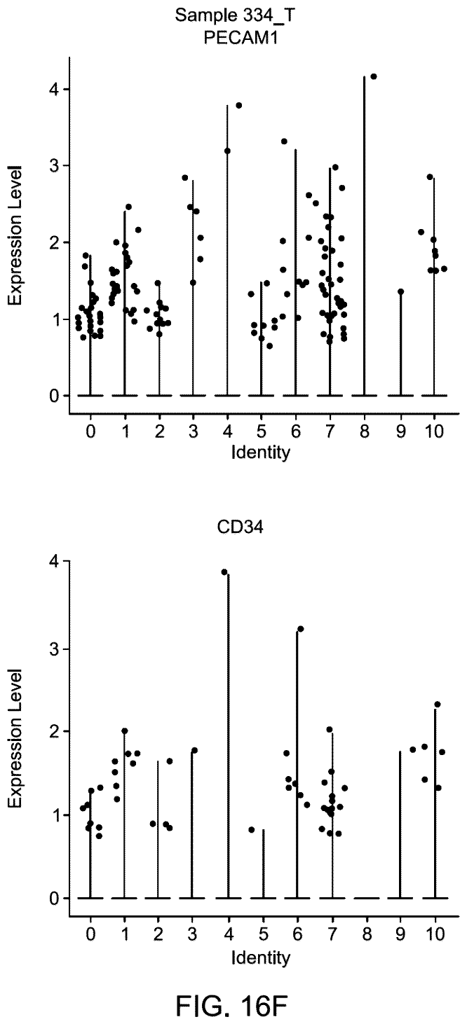

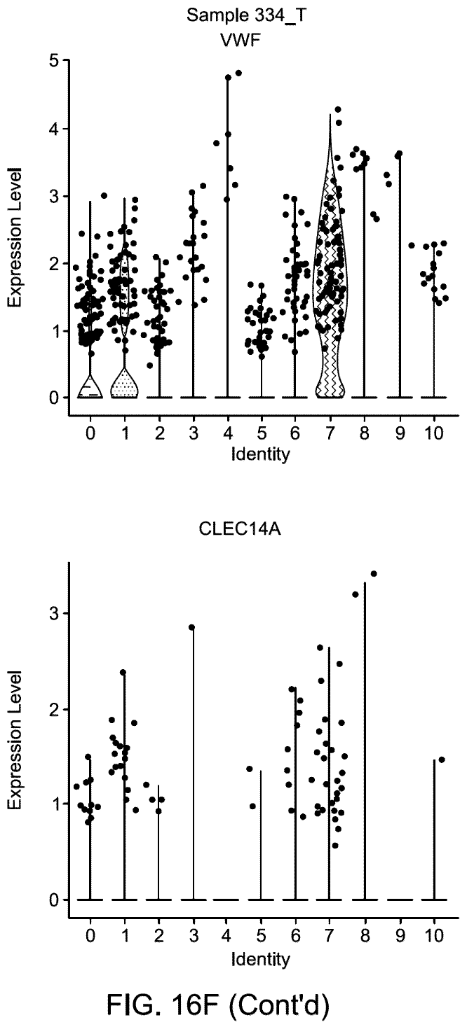

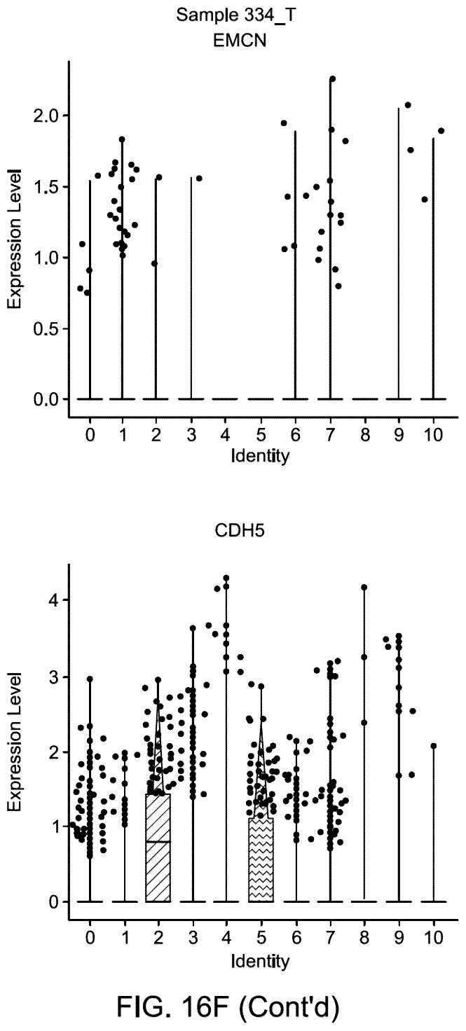

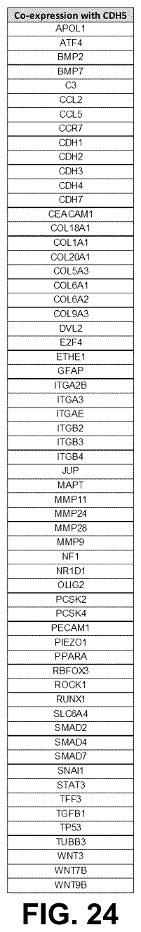

Spatial transcriptomics of GBM patient samples confirm CDH5 upregulation in tumor associated endothelium: The STRING analysis showed that CDH5 (VE-cadherin, CD144) is a major hub in the BTB-associated gene network. CDH5 is a calcium-dependent adherens junction with a fundamental role in maintaining BBB integrity. To further investigate its potential role in the BTB of GBM, we reanalyzed spatial transcriptomics data from malignant glioma tissue samples from Ravi et al (2022)⁴⁸ and confirmed the expression of CDH5 in comparison to matching non-tumorigenic brain cortex (FIG. 10A). This data showed considerable expression of CDH5 above cortex controls. CDH5 is spatially distributed across the tumor (FIG. 10B), and spatially clustered in comparison to non-tumorigenic brain regions across different clinical samples (FIG. 10C and FIG. 10D, and FIG. 16A, FIG. 16B, FIG. 16C). Indeed, CDH5 expression correlates with cell clusters expressing the endothelial markers PECAM1, CD34, VWF, CLEC14A and EMCN (FIG. 10E and FIG. 16D, FIG. 16E, FIG. 16F). CDH5 expression clustered and co-expressed with BBB-endothelial markers (CLDN5, VWF), extracellular matrix modulators (COL1A2, COL4A1, COL4A2, COL18A1, FN1 and MMP9) and the BBB amino acid transporter SLC7A5, and SLC38A5 (not shown). Moreover, we identified a list of 61 additional genes regionally co-expressed with CDH5 (FIG. 24, Supplementary Table 2), which includes CD31, and some other genes reported to modulate BTB permeability such as CCL2³⁴ and WNT7B.^{49,50} This supports the notion that CDH5 is highly expressed in tumoral vasculature and may be relevant to modulate BTB properties.

The spatial transcriptomic of GBM patient samples confirm CDH5 upregulation in tumor associated endothelium. (FIG. 10A) Surface plot of CDH5 expression from spatial transcriptomic performed on GBM tumors and non-tumorigenic cortex. (FIG. 10B) Spatial plot of CDH5 expression in cortex and tumor Ossue from pa0ent UKF_248. (FIG. 10C) UMAP plot from cortex and tumor indica0ng spatial CDH5 expression from (FIG. 10B) and, (FIG. 10D) integrated-clustered UMAP of the cortex and tumor transcriptional profiles (lea) and DimPlot of CDH5 expression (right). (FIG. 10E) Violin plot showing CDH5 and endothelial markers clustered-expression levels. (FIG. 10F) Gene Ontology biological processes enriched in the CDH5-expressing clusters, indicating vasculature development and regulation of angiogenesis as pathways involved. Data was obtained and re-analyzed from Ravi et al., (2022) using the SPATA2 package from R-studio.

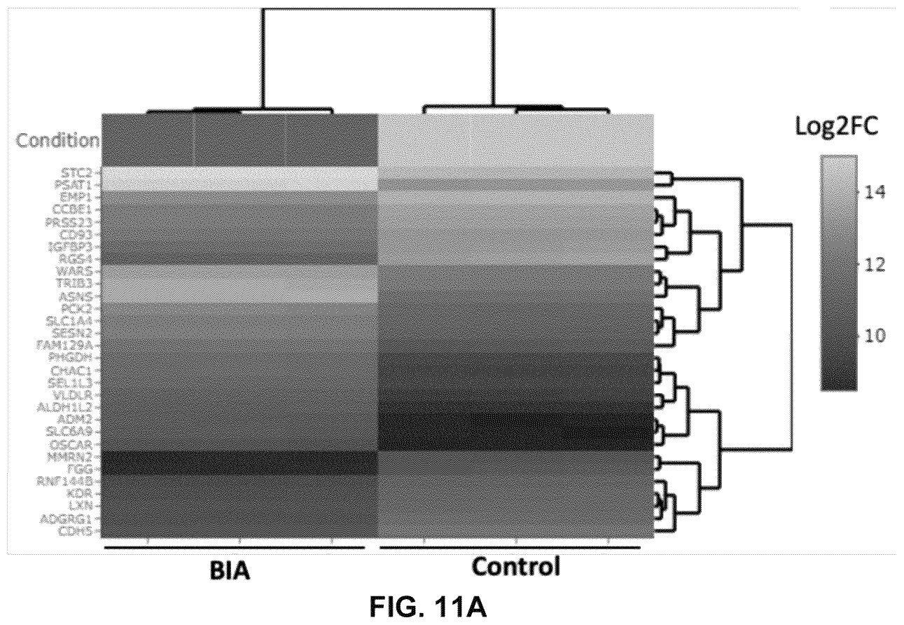

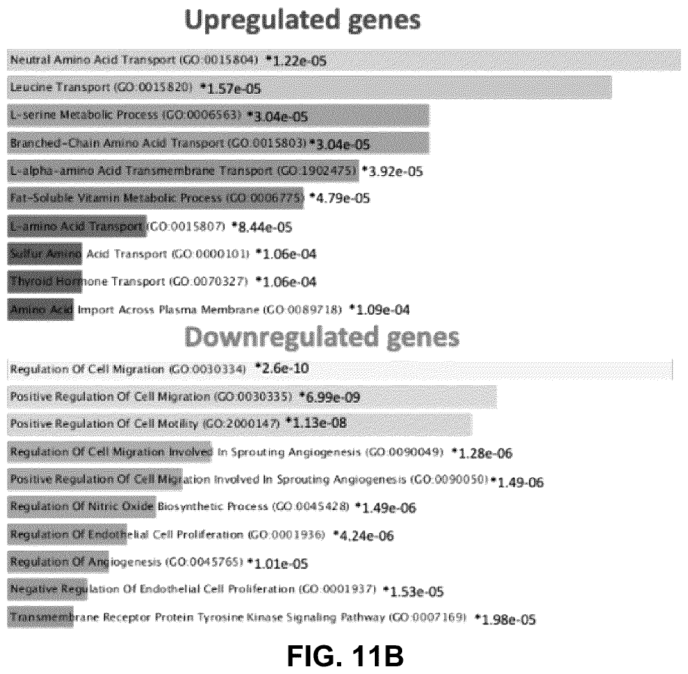

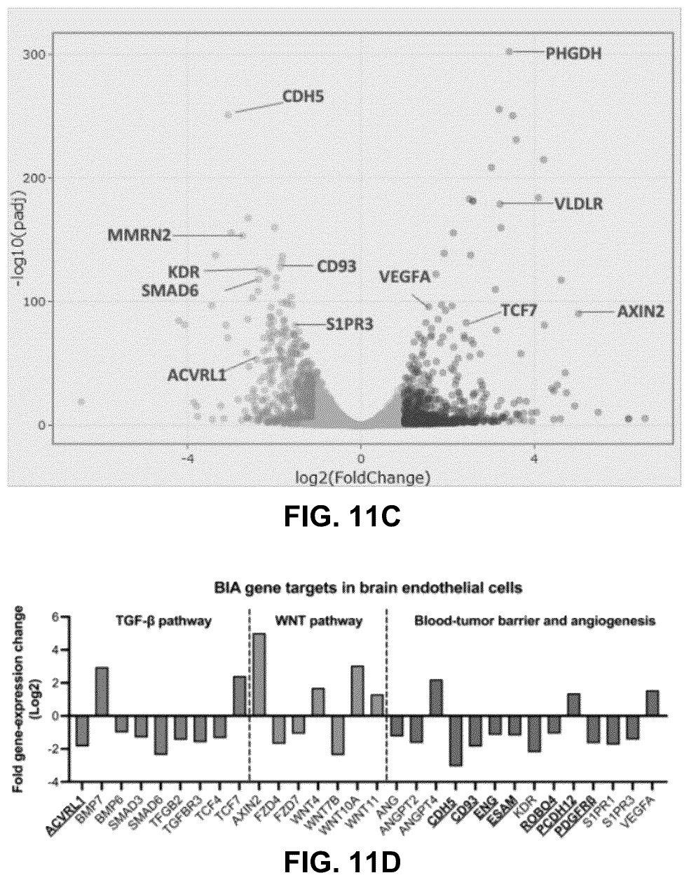

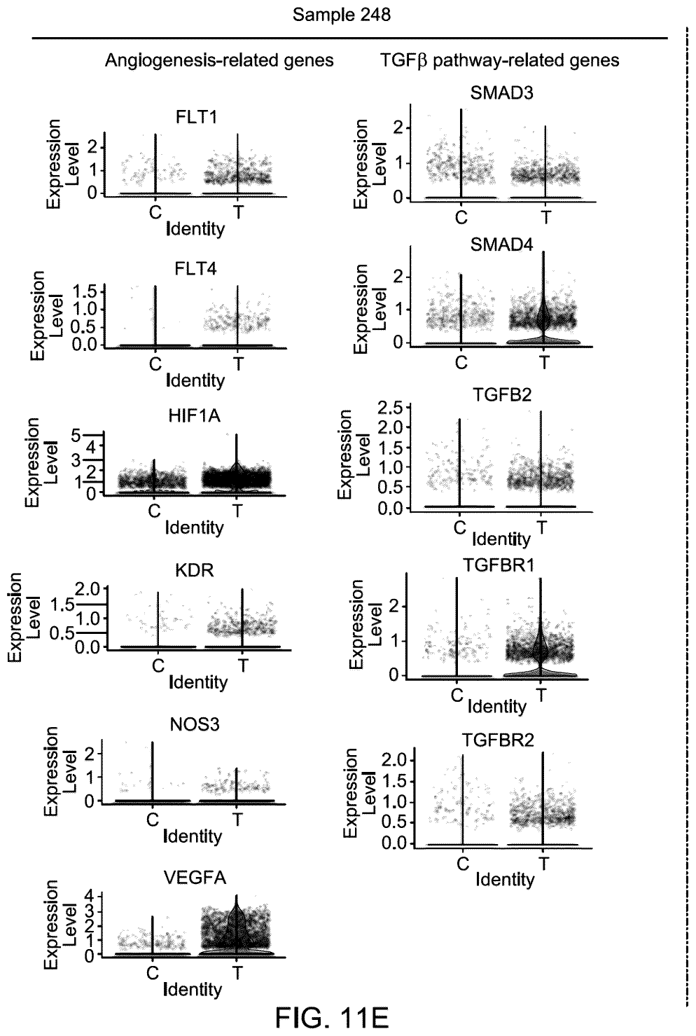

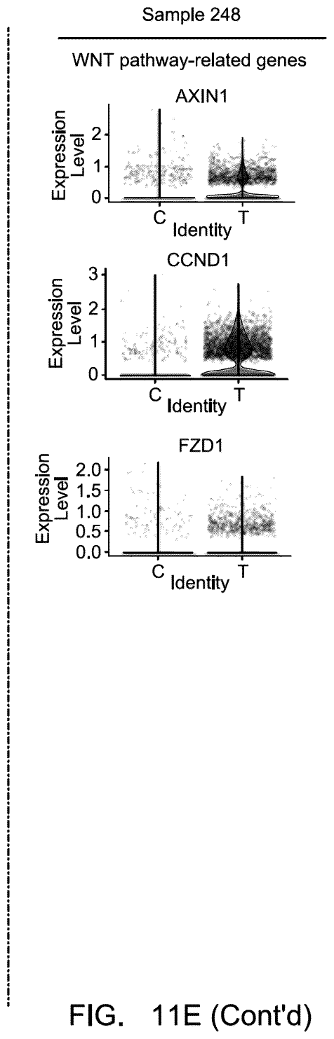

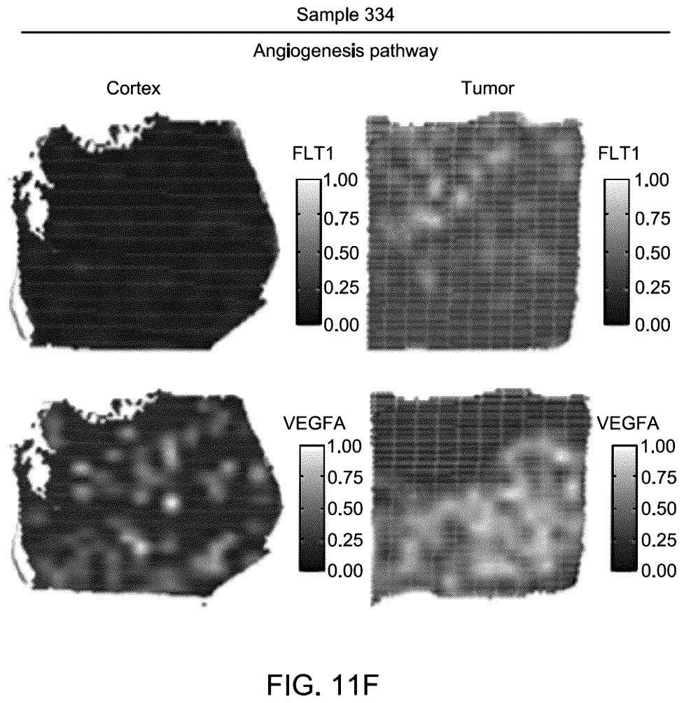

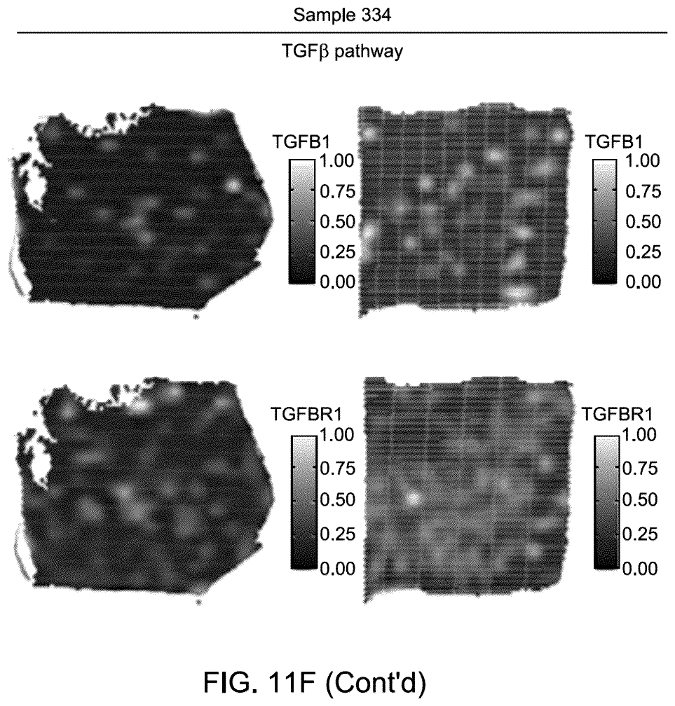

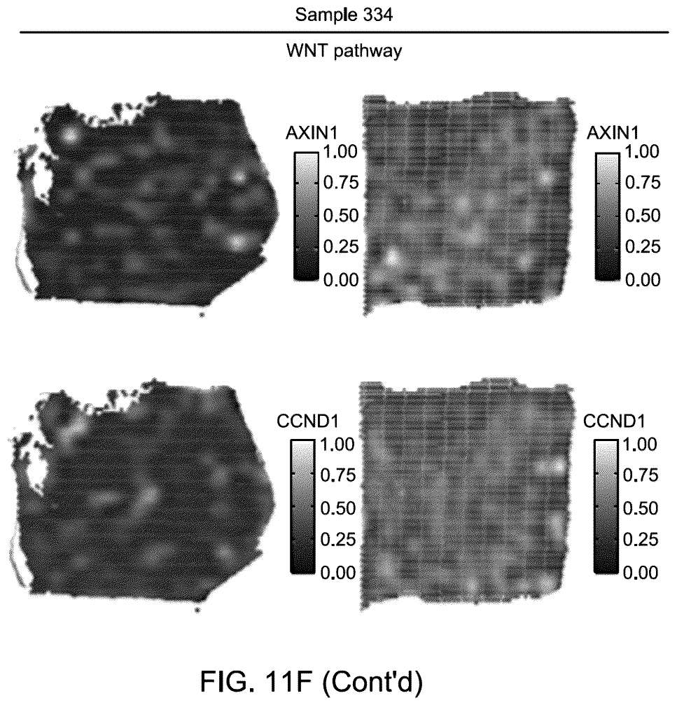

BIA targets angiogenesis and BTB-related transcrip&onal programs in brain endothelial cells: Previously we demonstrated BIA has anti-angiogenic effects in murine intra-cranial models of GBM.⁴⁵ This led us to investigate the transcriptional alterations associated with BIA treatment of brain endothelium. Bulk RNA-sequencing analysis of a well characterized human brain microvascular endothelial cell line, HCMEC/D3, treated with BIA showed considerable transcriptional dysregulation. The top 15 differentially expressed genes (DEGs) are displayed according to significance in a heatmap (FIG. 11A). Interestingly, CDH5 was one of the most downregulated genes upon BIA treatment (−3.06-fold, log 2). Gene Ontology (GO) analysis of significantly upregulated (862 genes) and downregulated (652 genes) differentially expressed genes revealed that BIA mostly induced expression of genes in processes related to amino acid transport. BIA also decreased expression of genes involved in annotated processes of cell migration, motility, angiogenesis, and endothelial proliferation, as well as nitric oxide synthesis and pathways of receptor tyrosine kinases (FIG. 111B). Volcano plot analysis (FIG. 11C) of expression log 2 fold-change vs. p-value significance of downregulated DEGs highlights CDH5 and other angiogenesis-related genes such as MMRN2, a direct interactor to CDH5, CD93, ACVRL1, KDR, SMAD6 and S1PR3. BIA also promoted expression of genes such as PHGDH, AXIN2, TCF7, VLDLR and VEGFA. Showing that genes BIA has broad effects on genes involved in diverse pathways. This led us to identify and arrange those dysregulated genes that are members of pathways involved in BBB permeability/integrity and in biological functions of angiogenesis and the BTB (FIG. 11D). Indeed, BIA modulates 8 of the 12 BTB-genes identified in the in-silico screening from clinical samples (FIG. 11D, highlighted). Most of these genes were downregulated by BIA, except PCDH12, which increased its expression. This finding suggests that BIA targets the expression of BTB-associated transcriptional programs in brain endothelial cells. Our spatial transcriptomic analysis confirmed the increased expression of Angiogenesis (FLT1, FLT4, KDR, VEGFA, NOS3 and HIF1A), TGF-β (SMAD3/4, TGFBR1/2, and TGFB2) and WNT (AXIN1, FZD1 and CCND1) pathways in GBM tumors above non-tumorigenic cortex (FIG. 11E). These genes were expressed across tumor samples (FIG. 11F), and indicate the relevance of these pathways in GBM.

BIA modulates vascular development, migration, L-serine metabolism, WNT and TGF-β pathways in brain endothelial cells. (FIG. 11A) Heat-map generated from the top-15 upregulated and downregulated genes from Bulk-RNA sequencing analysis performed on HCMEC/D3 cells treated with BIA (1 μM, 24 hrs). (FIG. 11B) Gene ontology analysis of >1.5 significantly upregulated and downregulated genes (Log 2 fold-change) by BIA in brain endothelial cells from (FIG. 11A). Biological processes are ranked by p-values, which are shown next to the GO designation. Analysis performed using the EnrichR software. (FIG. 11C) Volcano plot analysis from all the upregulated and downregulated genes by BIA. Labels on genes related to angiogenesis, TGF-β and WNT pathways are highlighted. (FIG. 11D) Gene expression fold-change (Log 2) levels of dysregulated genes by BIA related to the TGF-β and WNT pathways, angiogenesis and the tumor vascular associated genes (BTB-genes, highlighted). (FIG. 11E) Violin plots displaying expression levels of genes related to Angiogenesis, TGF-β and WNT pathways in sample UKF_248 which expression or activity are modulated by BIA. (FIG. 11F) Surface plot of genes related to angiogenesis, TGF-β and WNT pathways in sample UKF_334.

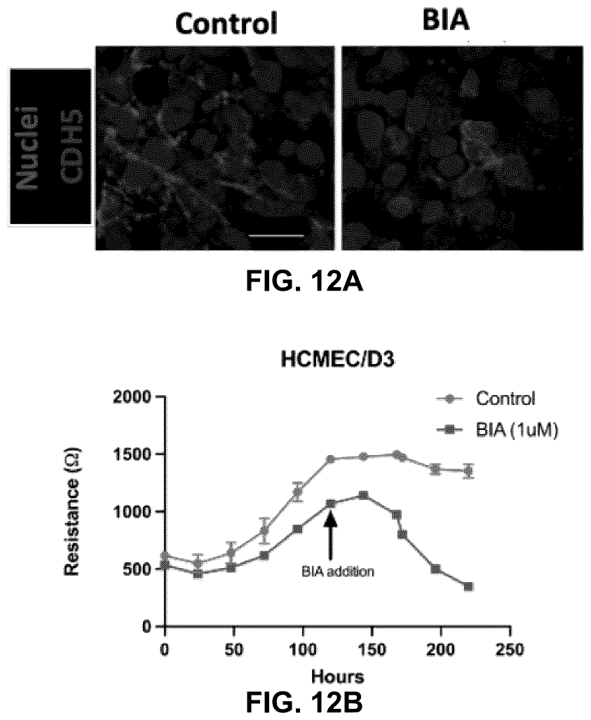

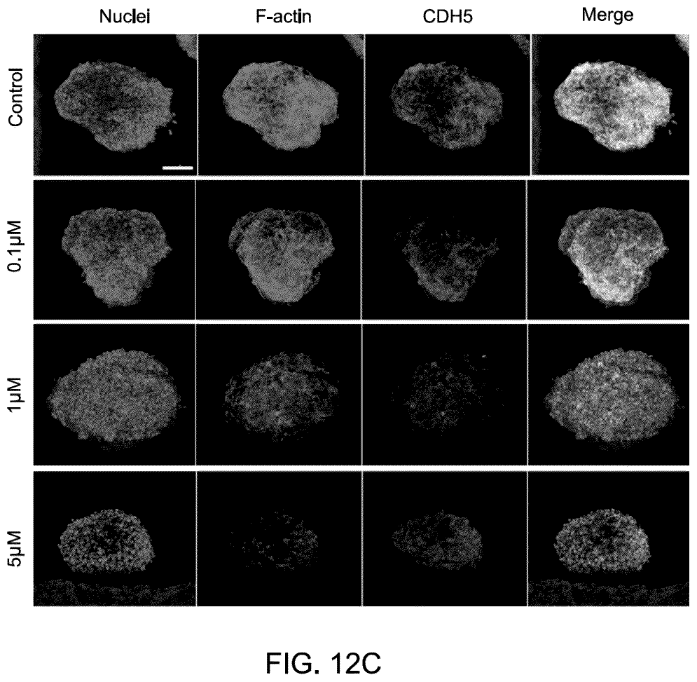

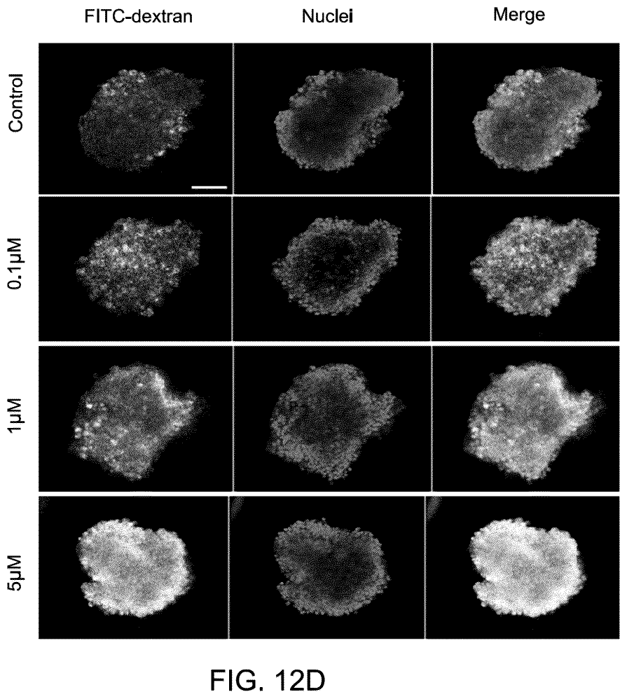

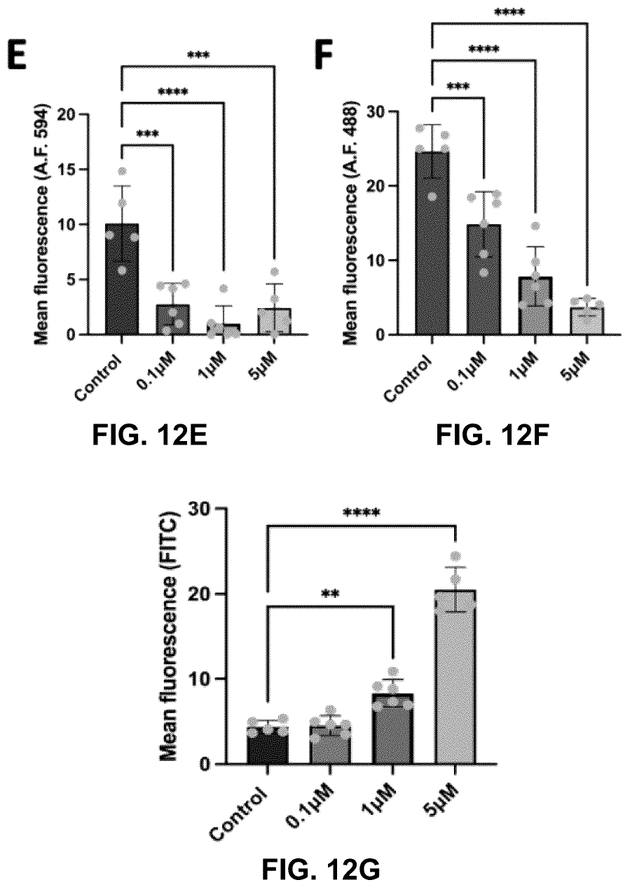

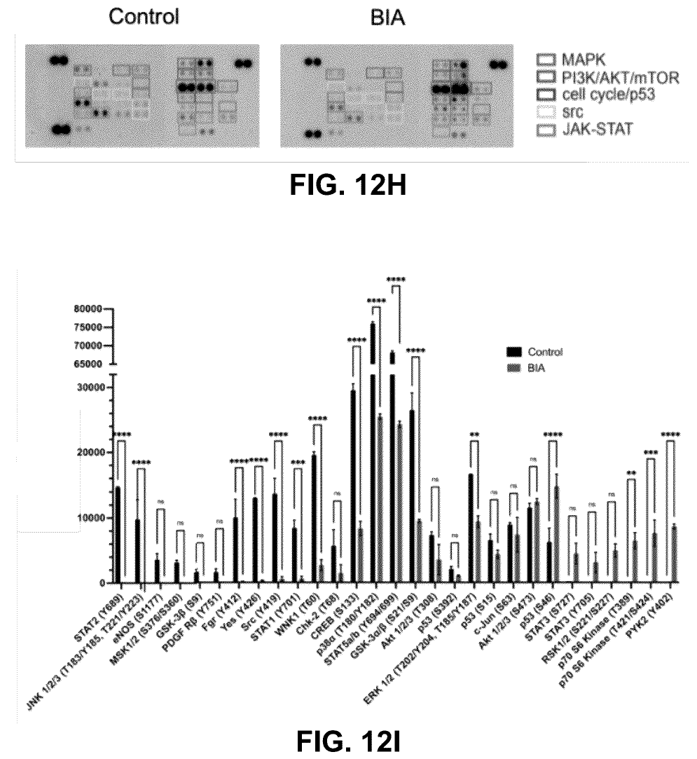

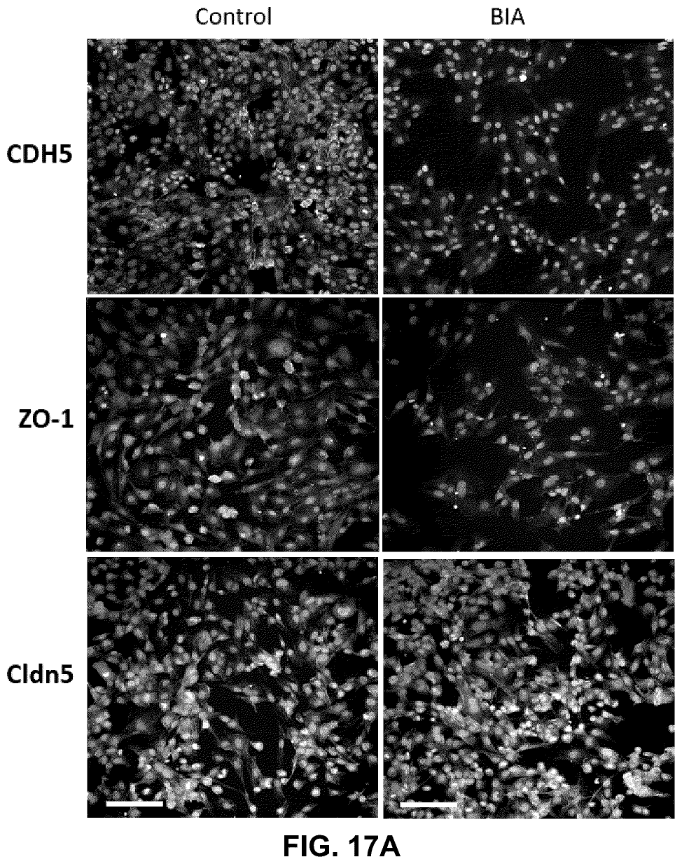

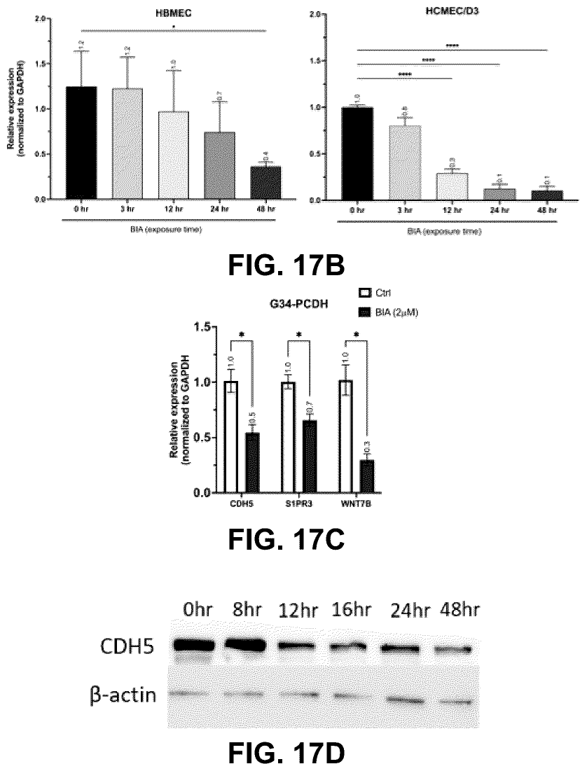

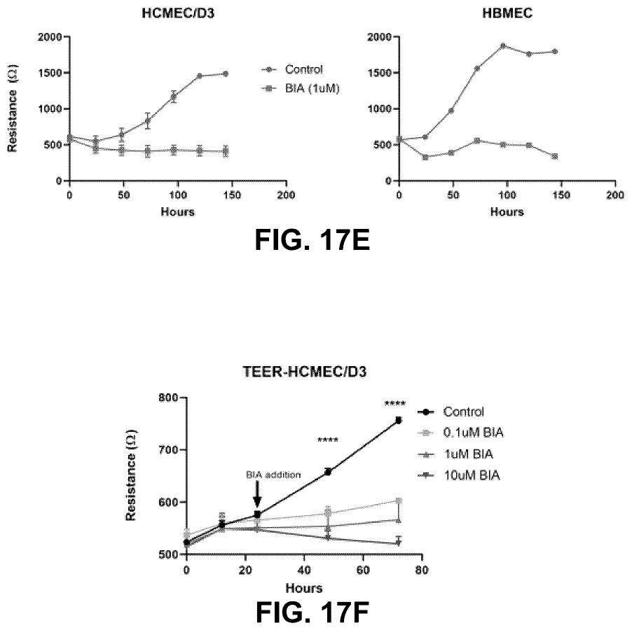

BIA disrupts barrier formation and increases permeability in BBB models in vitro: Given the importance of CDH5 to the BTB transcriptome, and its known role of maintaining vascular barrier integrity, we focused our efforts in further characterizing CDH5 expression in the BBB upon BIA treatment. Immunofluorescence (IF) staining of CDH5 showed a marked decrease at the membrane periphery in endothelial cells in vitro after treatment with BIA (FIG. 12A and FIG. 17A). We observed a marked reduction in ZO-1, but no difference for the BBB tight junction molecule Claudin-5. BIA decreased expression of CDH5 at the transcriptional level in two different brain endothelial cell lines, which remained in constant declining for up to 48 hours (FIG. 17B). Interestingly, BIA also reduced the expression of CDH5 in G34 GBM cells, with simultaneous diminishment of WNT7B and S1 PR3 expression, suggesting that BIA can modulate these endothelial barrier-related molecules in the tumoral context as well and is not restricted to vascular cells only (FIG. 17C). Protein levels of CDH5 reached maximum reduction at 12 hours post-BIA addition, which remained for 2 days (FIG. 17D).

To understand whether BIA might alter barrier formation properties in brain endothelial cells, we used a trans-endothelial electrical resistance (TEER) analysis of plated monolayers of HCMEC/D3. We added BIA to confluent monolayers and continued measuring TEER values in real time. Treatment with BIA led to a marked decreased of barrier formation by these cells (FIG. 12B). Moreover, addition of BIA 24 hours after plating prevented barrier establishment in two different brain endothelial cell lines (FIG. 17E). These effects occurred from 100 nM to 10 μM BIA (FIG. 17F), confirming that BIA can disrupt BBB integrity in vitro.

A loss of barrier integrity relates to leakiness and internalization of material across the endothelium. To determine if BIA can promote vascular permeability and material infiltration, we leveraged our in vitro multicellular BBB spheroid model.⁵¹ BIA decreased the expression of CDH5 in a dose-dependent manner (FIG. 12C and FIG. 12E) as shown by IF staining. F-actin was also reduced considerably (FIG. 12C and FIG. 12F). Incubation of a fluorescent-dextran (70 kDa) with the BBB spheroids treated with BIA showed a dose-dependent increase in permeability (FIG. 12D and FIG. 12G).

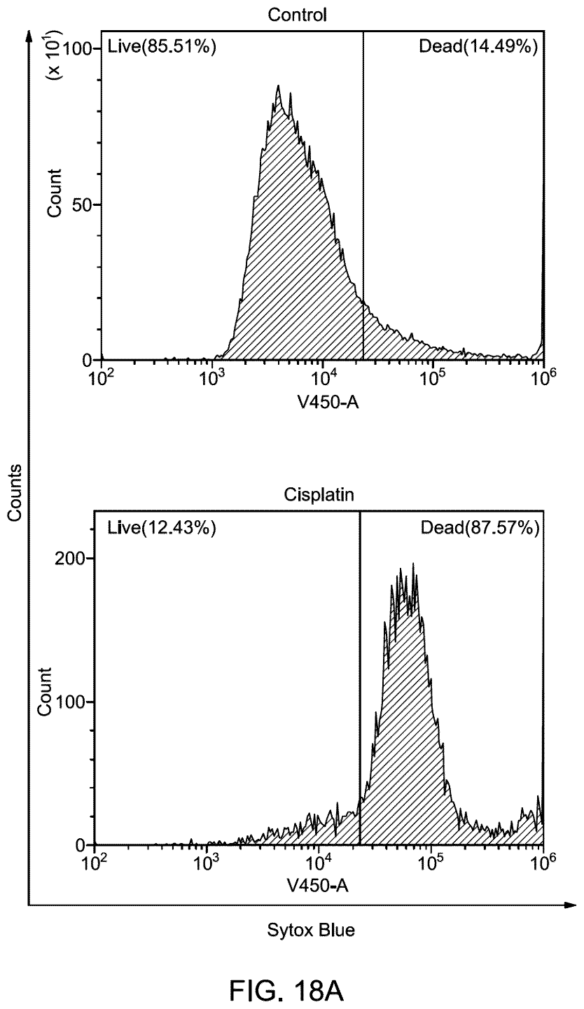

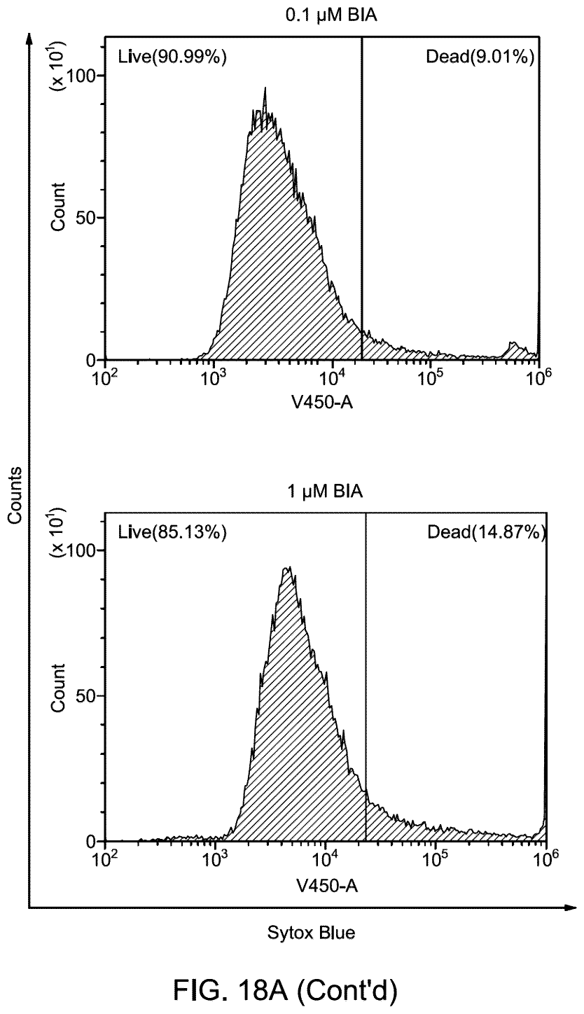

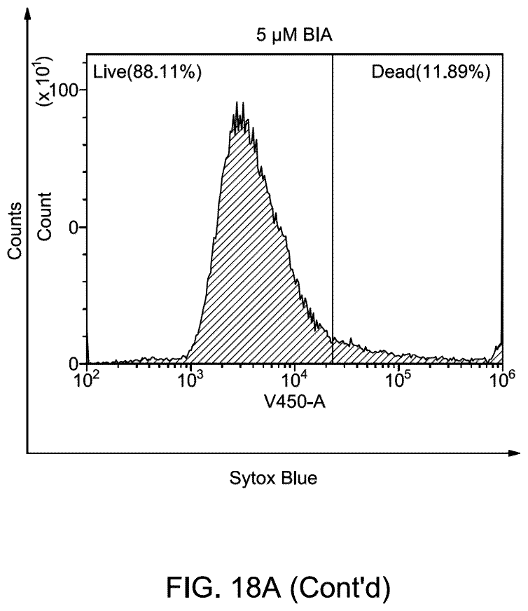

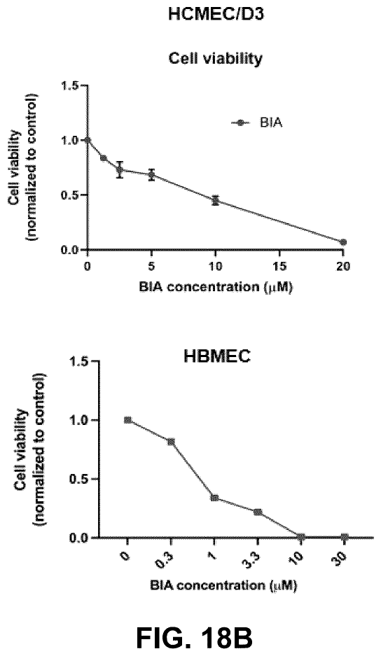

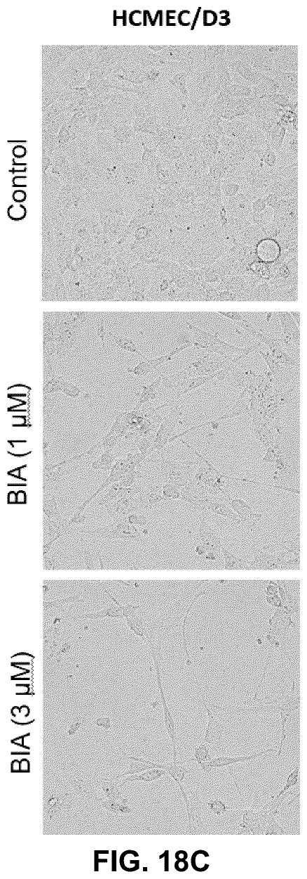

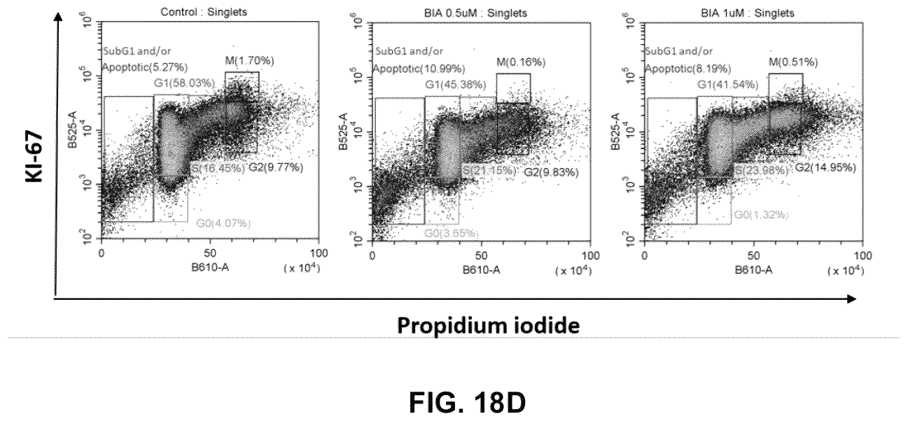

To understand whether the effects we observed are a consequence of endothelial cell death, we screened for apoptosis via flow cytometry, which did not show late apoptosis/necrosis at any of the used BIA doses in comparison to a cisplatin control (FIG. 18A). Viability was reduced up to 30% when treating within a ˜1-5 μM BIA range, and ˜50% and above for HBMEC cells treated at the same concentrations (FIG. 18B), indicating that BIA impacts endothelial cell metabolic vigor. Visual assessment of HCMEC/D3 cells treated with BIA did not show signs of apoptosis or necrosis, but an elongated phenotype with long filipodia (FIG. 18C), an interesting feature of endothelial cells treated with BIA. Cell cycle analysis via flow cytometry showed a slight decreased G1 and M states, indicating that BIA alters proliferative capacities of endothelial cells (FIG. 18D).

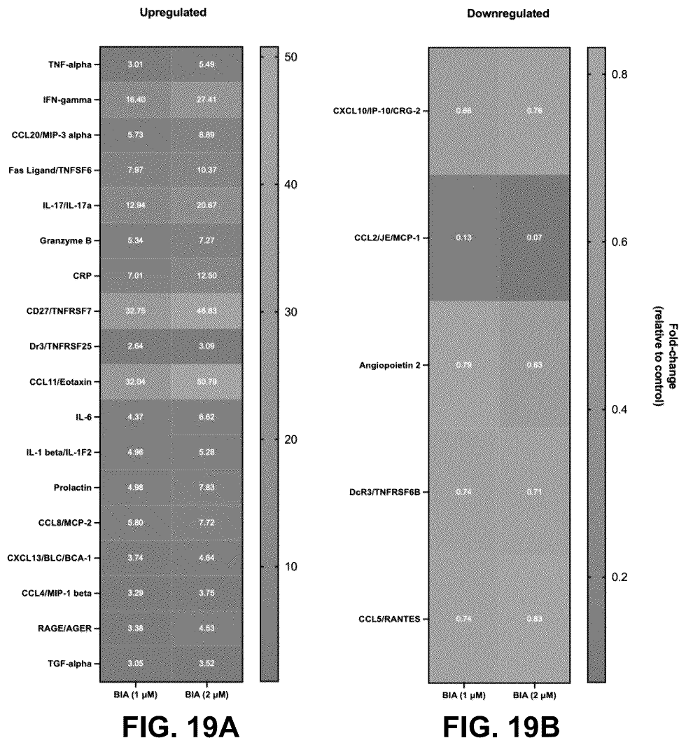

BIA targets several kinases in brain endothelium in vitro: To elucidate the kinase signaling pathways altered by BIA that could be involved in barrier modulation, we treated HCMEC/D3 cells with BIA and performed phospho-kinase array profiling (FIG. 12A and FIG. 12B). We observed a decrease of activating phosphorylation in members of the MAPK family (p38a, JNK1, MSK1/2 and ERK1/2), SRC family (SRC, YES, FGR) and transcription factors at activator sites (CREB, STAT1, STAT2, STAT5a/b and c-JUN). The MAPK and SRC pathways are known to control endothelial transcriptional programs through CREB and other transcriptional regulators.⁵²-5⁴ On the other hand, we observed phosphorylation of STAT3 at S727 and Y705, and in the p70 S6 kinase, which suggests activation of the mTOR pathway. Finally, secretome analysis of HCMEC/D3 cells treated with BIA indicates a pro-inflammatory secretion profile with an increase of cytokines such as TNF-α, IFNg, IL-17A, IL6, IL-1 b, prolactin, CCL8 and CCL4, among others (FIG. 19A). Whereas significant downregulation was seen to occur for CCL2 (FIG. 19B). Overall, our results indicate that BIA operates at different cellular signaling levels that induce diverse biological changes in brain endothelium, which might be required to induce the endothelial barrier disruption phenotype observed.

BIA prevents barrier formation by brain endothelial cells in vitro and increases dextran uptake in a three-dimensional BBB spheroid model. (FIG. 12A) Immunofluorescence staining of CDH5 with Alexa Fluor 594 (red) and nuclei with Hoechst 33342 (blue) in HCMEC/D3 cells treated with BIA (1 μM, 24 hrs). Representative image shown at 20×, scale bar=20 μm. (FIG. 12B) TEER assay resistance values of HCMEC/D3 treated with BIA upon monolayer confluence (capacitance ˜10 nF). Time-point of BIA addition is indicated (˜110 hrs.). (FIG. 12C) Confocal microscopy immunofluorescence images of BBB spheroids treated with indicated doses of BIA for 72 hours. Staining of F-actin with Phalloidin-Alexa Fluor 488 (green), CDH5 with Alexa-Fluor 594 (red) and nuclei with Hoechst 33342 (blue). Maximal projection intensity is shown from z-stack images (5 μm depth, 20 layers). Pictures taken at 10×, scale bar=100 μm. (FIG. 12D) FITC-conjugated dextran (70 kDa) permeability assay in BBB spheroids. Dextran (gray) and nuclei stained with Hoechst 33342 (purple) are shown. Maximal projection intensity is shown from z-stack images (5 μm thick, 20 layers). Pictures taken at 10×, scale bar=100 μm. Mean fluorescence quantification of nuclei, (FIG. 12E) CDH5, (FIG. 12F) Phalloidin and (FIG. 12G) FITC-dextran from images in (FIG. 12C) and (FIG. 12D) using the Image J (Fiji) soaware. Data shows individual values, mean and standard deviation, n=4-5. Ordinary one-way ANOVA for statistical significance. **p=0.0028, ***p=0.008, ****pti0.0001. (FIG. 12H) Human phospho-kinase array of HCMEC/D3 cells exposed to 1 uM BIA for 24 hours. Colored squares highlight wells related to the indicated pathways. Samples were analyzed in duplicates. (FIG. 12I) Quantification of signal by Image J (Fiji) of dot-blot shown in (FIG. 12H). Mean and standard deviation of duplicates are shown. Two-way ANOVA analysis was performed. **p=0.0015, ***p=0.005, ****pti0.0001.

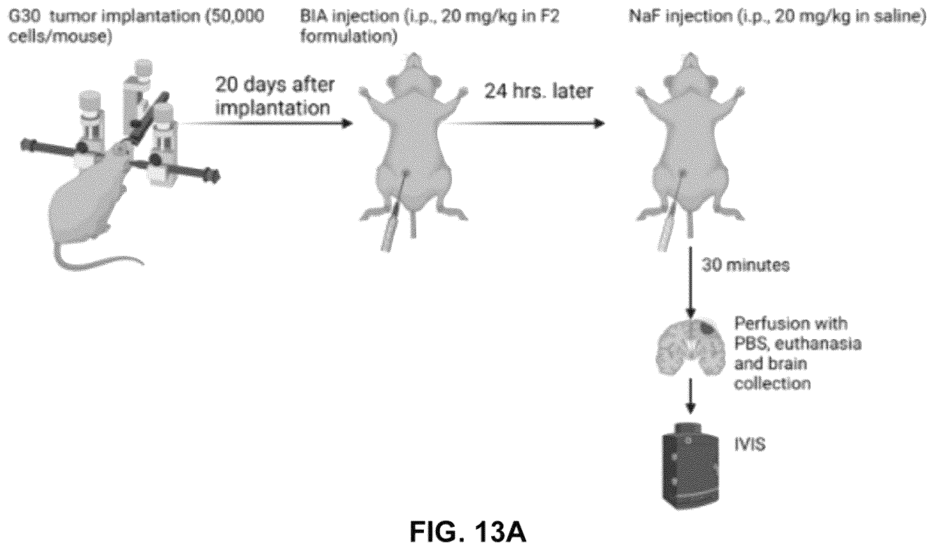

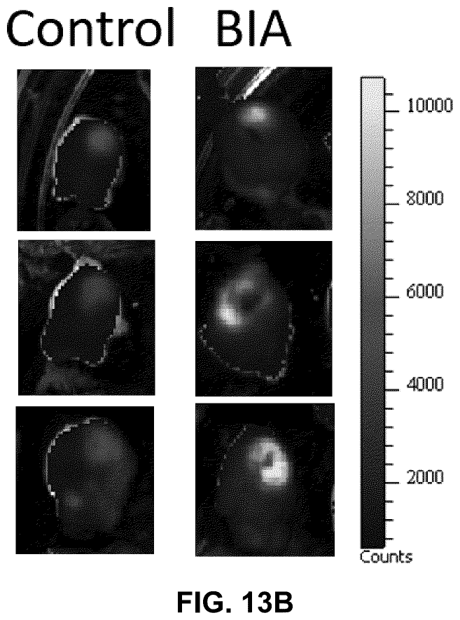

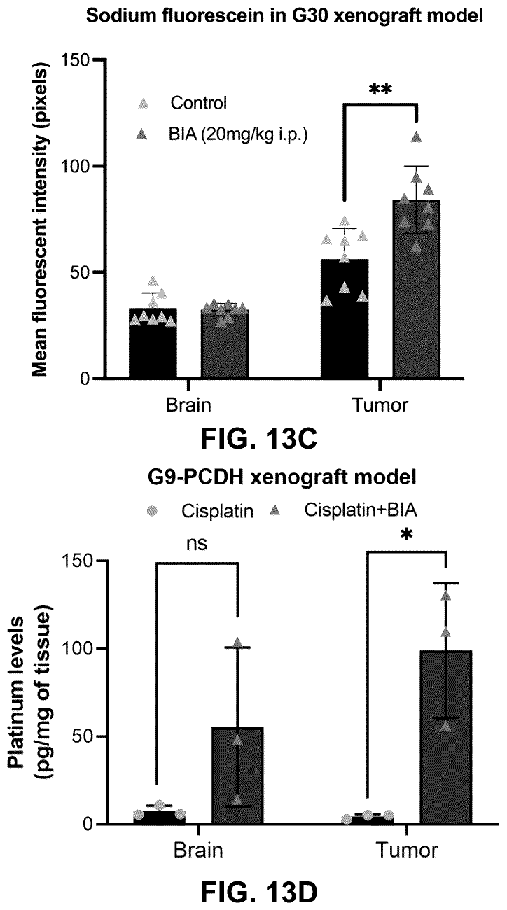

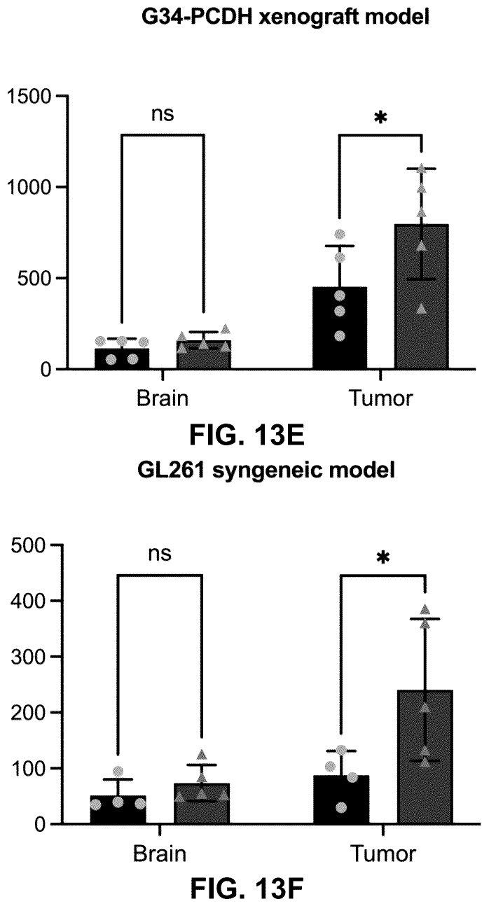

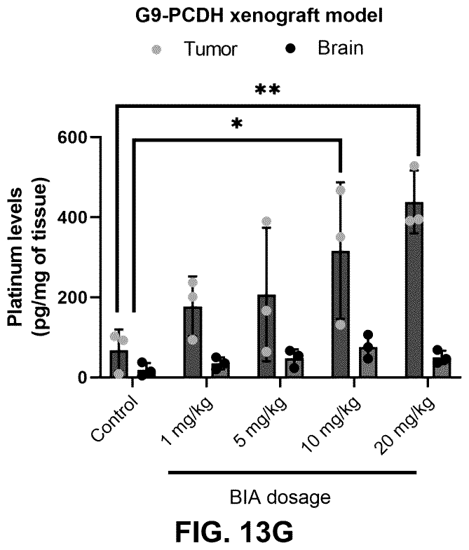

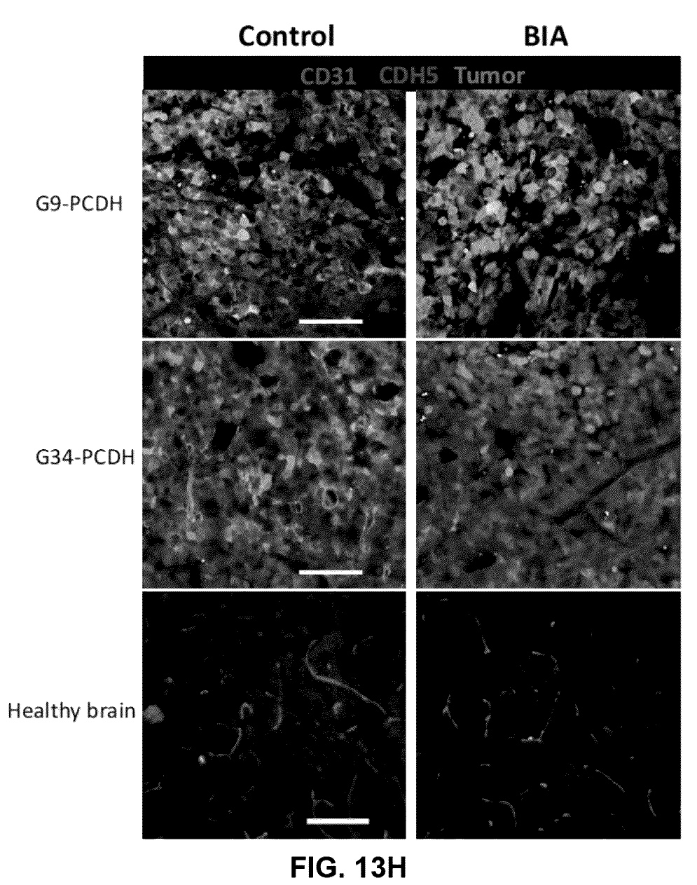

BIA increases intra-tumoral drug accumulation in murine intra-cranial models of GBM: To understand whether BIA could also increase permeability in the BTB under a GBM context in vivo, we implanted patient-derived GBM cells (G30) in nude mice and treated them with BIA and administered NaF as indicated in FIG. 13A. Increased accumulation of NaF within the tumor was observed amer BIA administration, in comparison with untreated controls (FIG. 13B). Quantified fluorescent signal showed significant accumulation in the tumor, but not in healthy brain, suggesting that BIA administration promoted intra-tumoral uptake of NaF.

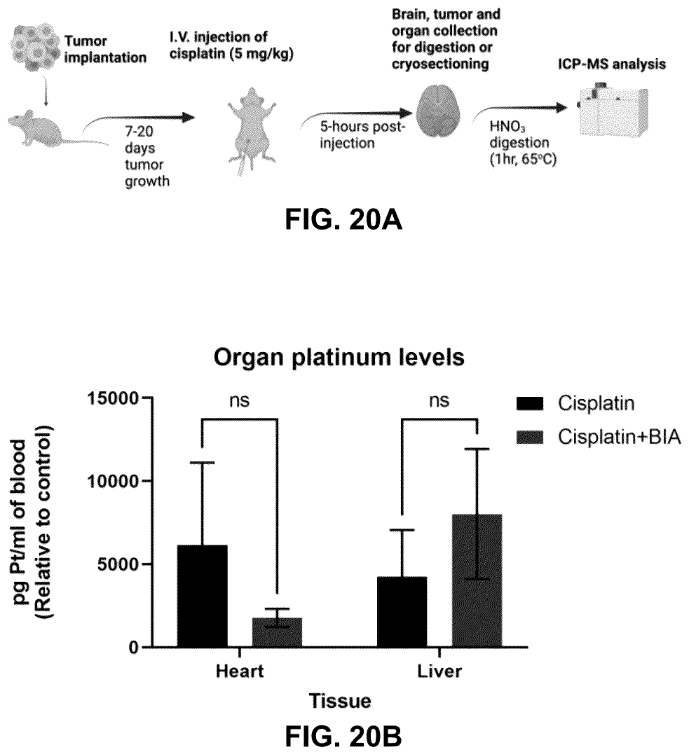

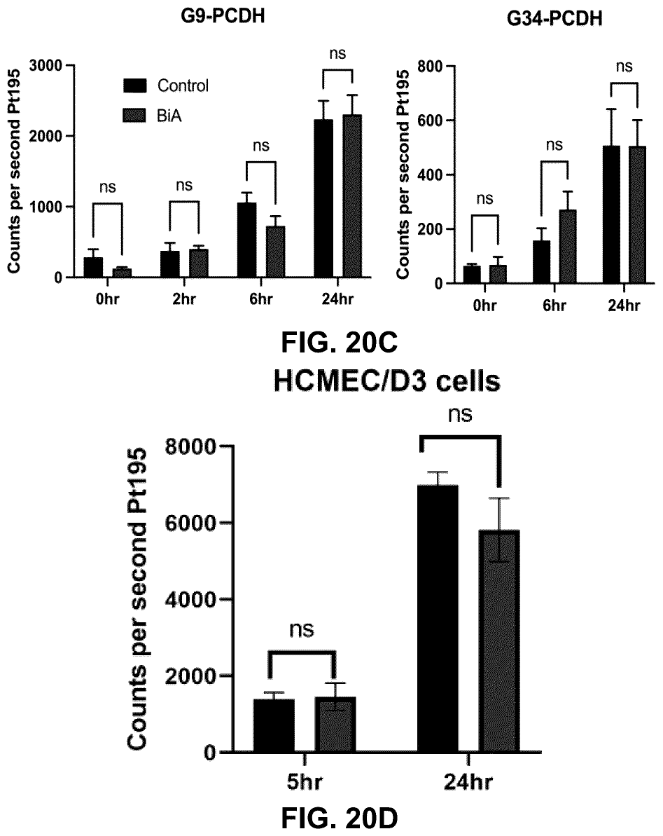

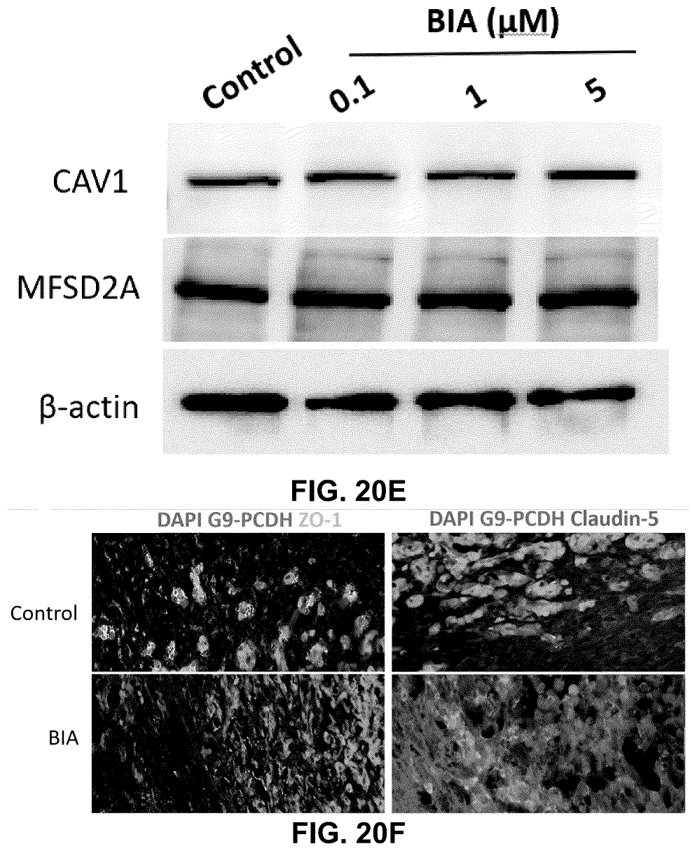

We then interrogated whether the non-BBB penetrant chemotherapeutic drug cisplatin, would increase its accumulation in the tumor, but not surrounding tissue, due to BIA. For this, we injected 5 mg/kg of cisplatin and allowed circulation in the system for 5 hours We collected processed the tissue downstream (see Materials and methods 2 section) for Inductively Coupled Mass-Spectrometry (ICP-MS) analysis-based platinum quantification. (FIG. 20A). Pre-treatment of BIA permitted significant cisplatin intra-tumoral accumulation in patient-derived (FIG. 13D and FIG. 13E) and syngeneic murine GBM tumors (FIG. 13F). Importantly, no significant difference of uptake was seen in contralateral healthy brain regions, indicating that BIA acts selectively in the tumor but not in the brain. Moreover, no difference in platinum accumulation was seen in peripheral tissues such as heart or liver, thereby supporting the notion that BIA selectively increases cisplatin uptake in tumor but not healthy tissue (FIG. 20B).

Further studies showed that the uptake of cisplatin is dependent on the dose of BIA (FIG. 13G). Indeed, administration of BIA above 10 mg/kg already showed significant cisplatin accumulation in the tumor. To test possible mechanisms of how BIA operates in augmenting drug accumulation in tumors, we treated GBM cells (FIG. 20C) and brain endothelial cells (FIG. 20D) with BIA and cisplatin simultaneously. In either case, we did not observe any advantage in drug accumulation due to BIA addition, suggesting that direct cellular internalization is not a mechanism of operation for BIA. In fact, treatment of endothelial cells with BIA did not show any changes of protein levels of CAV1 or MFSD2A (FIG. 20E), important molecular actors in endocytosis and transcytosis in the BBB.

Next, we evaluated CDH5 expression in our patient-derived xenograft GBM models and its potential alterations upon BIA treatment. Administration with BIA showed a striking decrease of CDH5 in CD31+endothelial cells and in CDH5+ tumor cells 24 hours after treatment (FIG. 13H and FIG. 13I). On the other hand, we did not observe significant changes in expression of CDH5 in contra-lateral healthy brain regions, correlating with the observation that increased drug delivery effects due to BIA are mostly tumoral endothelium-specific. Additionally, we assessed the expression of ZO-1 and Claudin-5 in these tissues. We observed mild reductions of ZO-1 expression as well, but no visible differences in Claudin-5 staining (FIG. 20F). Collectively, these data provide evidence that BIA selectively targets the tumoral vasculature at the BTB, which downregulates CDH5 expression, disrupting tight junction formation and increasing accumulation of chemotherapy in GBM murine tumors.

Systemic administration of BIA increases the selective uptake of sodium fluorescein and platinum chemotherapy in GBM murine tumors. (FIG. 13A) Workflow schematic of BIA administration and subsequent injection of NaF for BTB permeability assessment. (FIG. 13B) In vivo imaging system (IVIS) pictures of G30-tumor bearing brains from mice injected with BIA and NaF as shown in (FIG. 13A). (FIG. 13C) Quantification of image intensity was performed with Image J (Fiji). Mean and standard deviation are shown, n=7-8. Unpaired t-test for statistical significance, **p=0.0024. (FIG. 13D) Platinum quantification via ICP-MS of brain and tumor tissue from tumor-bearing mice injected with cisplatin in G9-PCDH, (FIG. 13E) G34-PCDH and (FIG. 13F) GL-261 murine models. Cisplatin (5 mg/kg) was administered 24 hours aaer BIA injection. Mean and standard deviation are shown, n=3-5/group. Two-way ANOVA statistical analysis was performed, *pti0.05. (FIG. 13G) Platinum quantification via ICP-MS of tumor and brain tissue of a G9-PCDH tumor-bearing xenograa model administered with increasing BIA doses. Mean and standard deviation are shown, n=3/group. Two-way ANOVA test, *p=0.0177, **p=0.0016. (FIG. 13H) Confocal immunofluorescence imaging from frozen and sectioned brain tissue from G9-PCDH and G34-PCDH xenograa murine models, 24 hours aaer injection with 20 mg/kg of BIA. CDH5 was stained in tumor and healthy brain tissue with Alexa Fluor 594 and blood-vessels with anti-CD31 and Alexa Fluor 405. GBM cells are pre-labelled with GFP. Images shown at 20×, with scale bars at 100 μm, accordingly. (FIG. 13I) CDH5 fluorescence quantification (Alexa Fluor 594) from experiment in (E) using Image J (Fiji). Mean and standard deviation are shown. Unpaired t-test (n=3/group). ** p=0.0013, *p=0.0334.

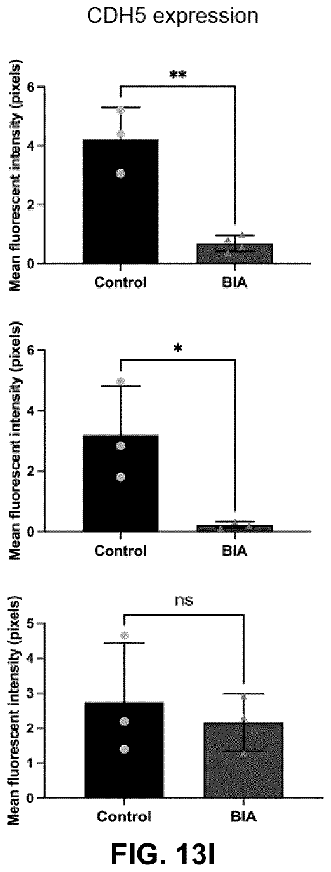

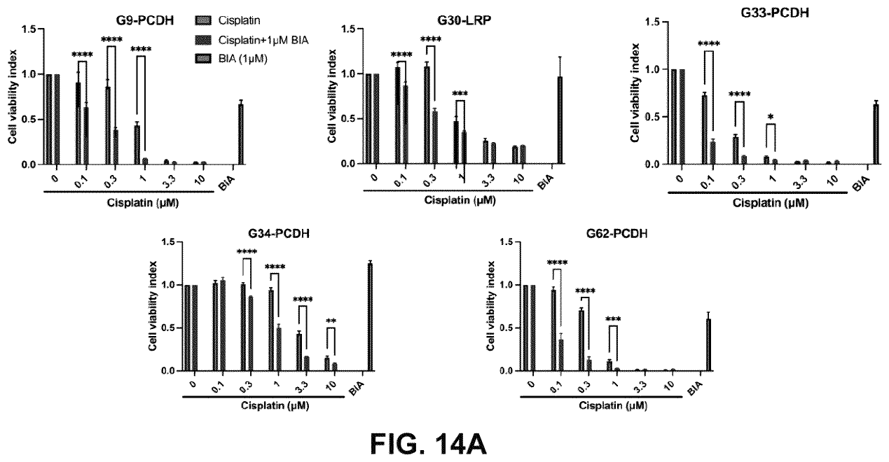

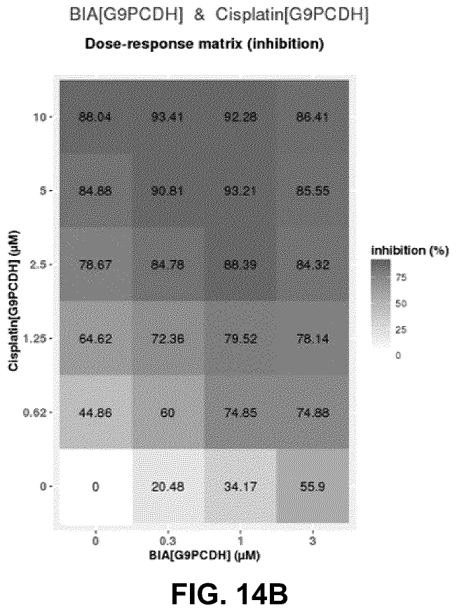

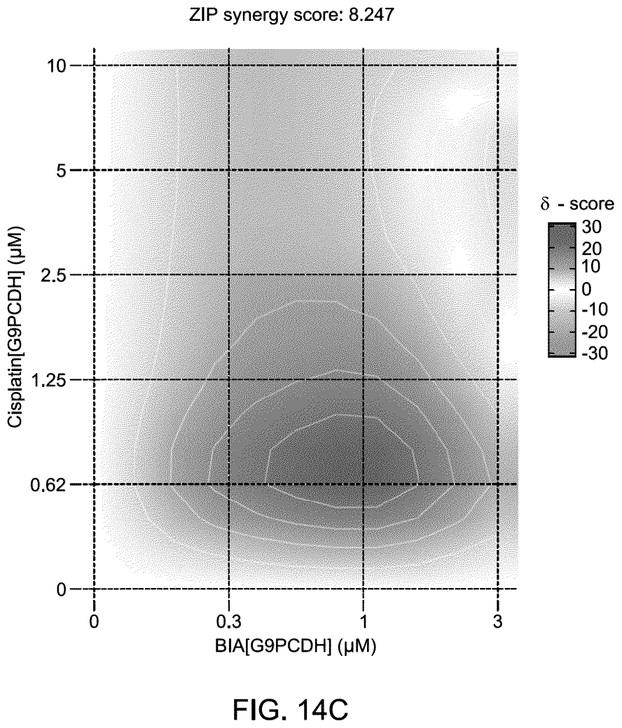

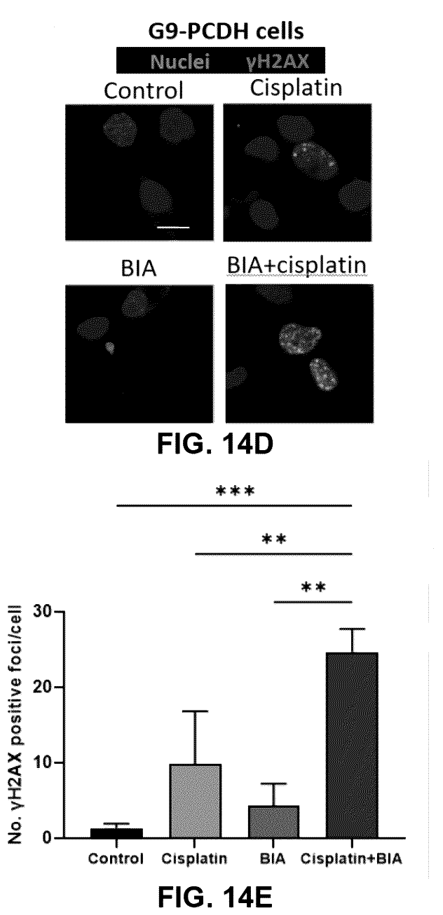

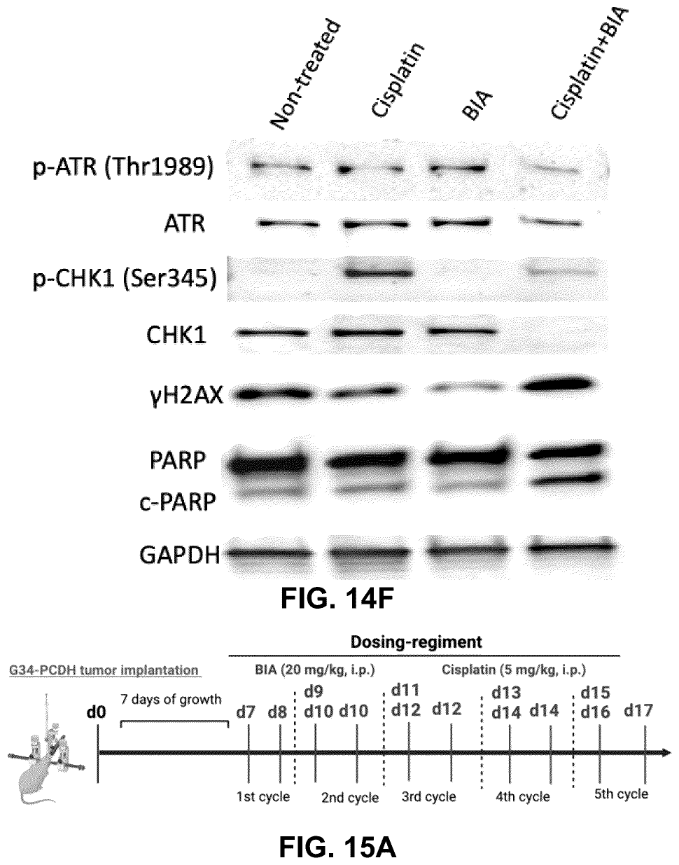

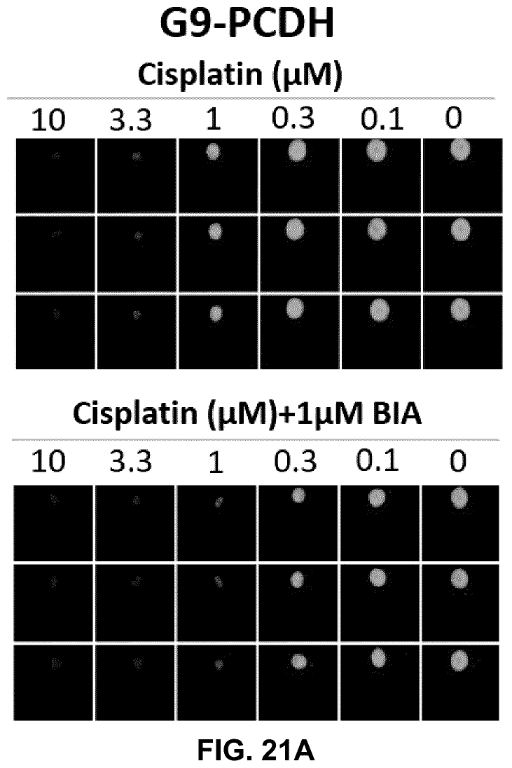

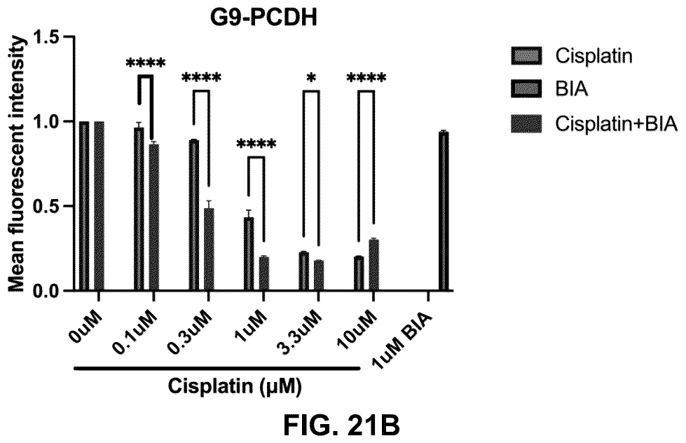

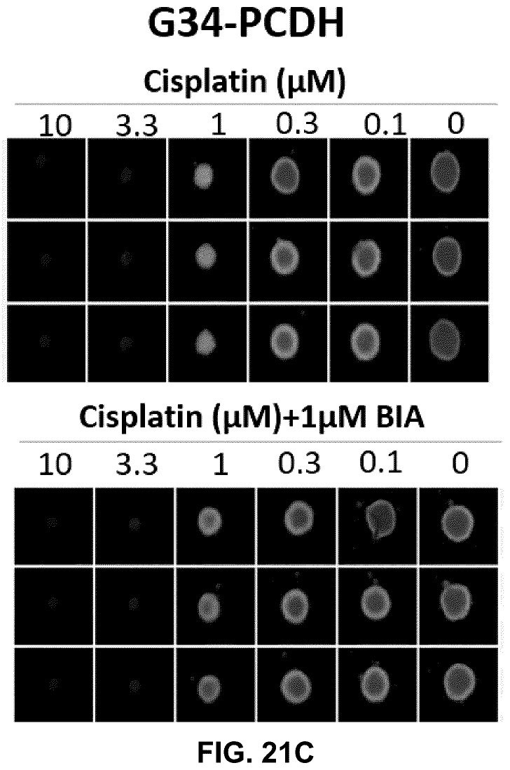

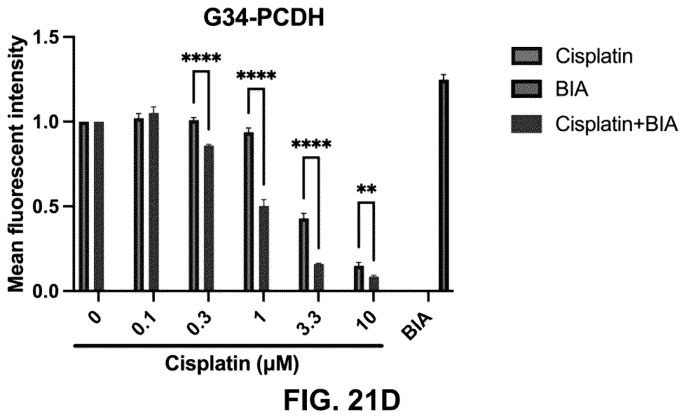

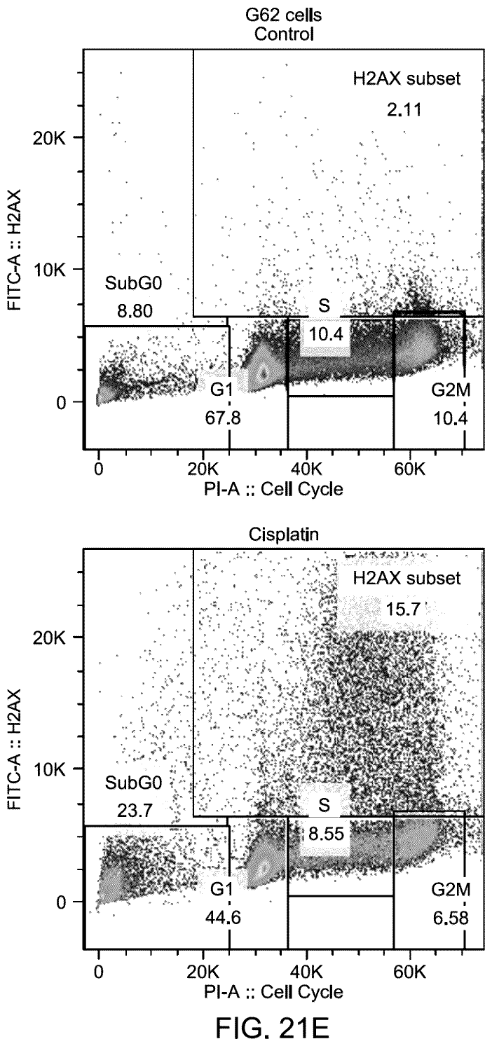

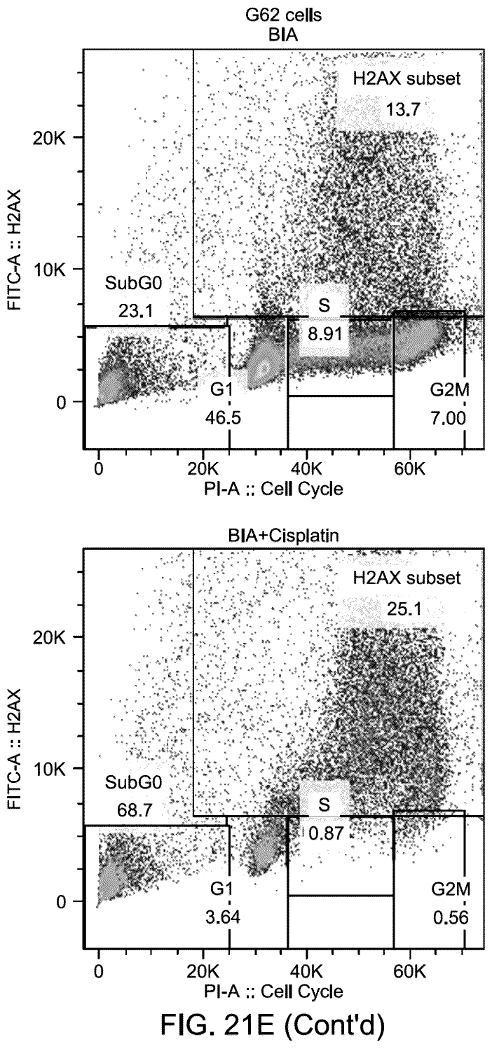

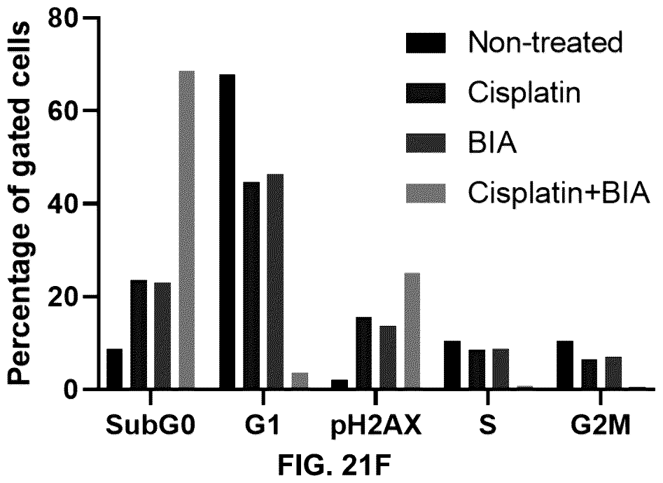

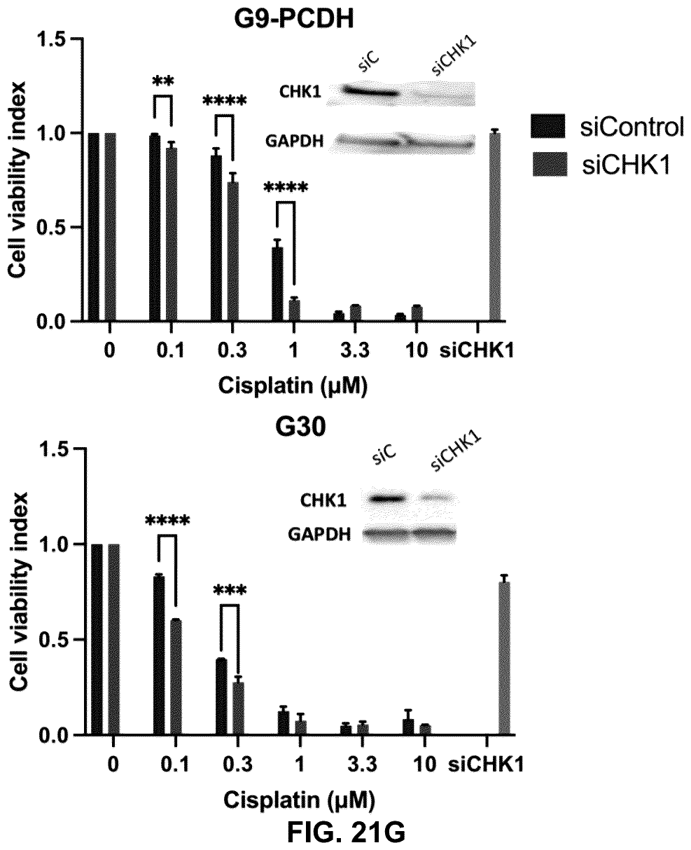

BIA potentiates cisplatin cytotoxicity by fostering its DNA-damage capacity in GBM cells: We also questioned whether BIA and cisplatin in combination could also show a therapeutic advantage than administration of each agent alone. This thought was based on several reports showing cytotoxic synergism of small-molecule kinase inhibitors in combination with cisplatin in cancer.⁵⁵⁻⁵⁷ Accordingly, we cultured a panel of patient-derived GBM neuro-spheres and treated with BIA and cisplatin combination, with single-treatment groups as controls (FIG. 14A). Using a cell viability assay, we observed that combination of BIA and cisplatin dramatically increased the cytotoxic power of each of these compounds alone. Best combinatorial effects were significant at cisplatin ˜1 μM and below. BIA single-treatment controls reduced the cell viability of cells mildly. In accordance, this BIA/cisplatin combination decreased the neuro-sphere formation capacity and growth of G9 and G34 cells (FIG. 21A, FIG. 21B, FIG. 21C, FIG. 21D). To identify potential synergistic interactions between BIA and cisplatin, we utilized the cell viability data in the SynergyFinder 3.0 software. BIA potentiated cisplatin inhibition capacity (overall d-score=8.24), mainly at doses at 2.5 μM and below (FIG. 14B). We also identified high likelihood of synergism (highlighted area, d-scores>10) at the lower doses for cisplatin (˜0.6 μM-2.5 μM) in combination with all tested BIA doses (FIG. 14C). Interestingly, at the upper cisplatin dose-ranges, its interaction with BIA remained non-synergistic. Thus, cisplatin and BIA in combination show synergistic anti-glioma cytotoxic effects.

Next, we assessed the DNA damage levels of the BIA/cisplatin combination by IF imaging of γH2AX nuclear foci. Indeed, the BIA/cisplatin combination significantly augmented the frequency of γH2AX foci in the nucleus of GBM cells above single-treatments and non-treated controls (FIG. 14D and FIG. 14E). This increase in γH2AX events due to the BIA/cisplatin combination was also observed by flow cytometry, which correlated with loss of cell cycle progression (FIG. 21E and FIG. 21F). We evaluated the phosphorylation and protein levels of the ATR/CHK1 axis, important regulators of the DNA damage response during cisplatin exposure.⁵⁸ Simultaneous exposure of BIA and cisplatin lowered activation phosphorylation of ATR (Thr1989), and interestingly, reduced the expression of CHK1 and its activation (Ser345) greater than single-treatment controls. In turn, γH2AX levels and cleavage of PARP were induced upon this combination (FIG. 14F). Given the strong depletion of the ATR/CHK1 axis activity, we performed siRNA-dependent knock-down in our GBM cell lines. Use of siCHK1 increased the susceptibility of these cells to cisplatin titrations, mainly at concentrations below 1 μM (FIG. 21G), supporting the notion that targeting of the ATR/CHK1 axis is an important factor in the BIA-induced potentiation of cisplatin cytotoxicity.

BIA potentiates platinum-based cytotoxicity by targeting DNA-repair pathways in patient-derived GBM cells. (FIG. 14A) GBM cell viability assay (ATP-based) using Cell-Titer Glo 3D of BIA and cisplatin combination treatment. Cisplatin doses are indicated in x-axis, BIA remained at a constant concentration of 1 μM. Cells were treated for 5 days and analyzed using a plate reader for luminescence quantification. Mean and standard deviation are shown, n=3/group. (FIG. 14B) Immunofluorescence staining of γH2AX (Alexa-Fluor 647) in G9-PCDH cells treated with 1 μM of cisplatin and/or BIA, for 72 hours. Nuclei were stained using Hoechst 33342. Representative image of 5 pictures per condition. Pictures taken at 40×, scale bar=20 μm. (FIG. 14C) Quantification of γH2AX foci from (FIG. 14B) using Image J. (FIG. 14D) Western blot of G9-PCDH cells treated with 1 μM of cisplatin and/or BIA, for 72 hours, probing for the ATR/CHK1 axis proteins. GAPDH was used as loading control and cleaved-PARP as a cell-death marker. Representative image from triplicate experiments. (FIG. 14E) Dose-response matrix showing inhibition percentage of BIA and cisplatin combinations at various concentrations using SynergyFinder 3.0. G9-PCDH cells were treated and viability analyzed as indicated in the Cell viability assay section (see Materials and methods 2). (FIG. 14F) ZIP method synergy score of BIA and cisplatin combinations. The overall average 6-score is indicated on top of the chart. The dose combinations showing an increased likelihood of synergy is highlighted.

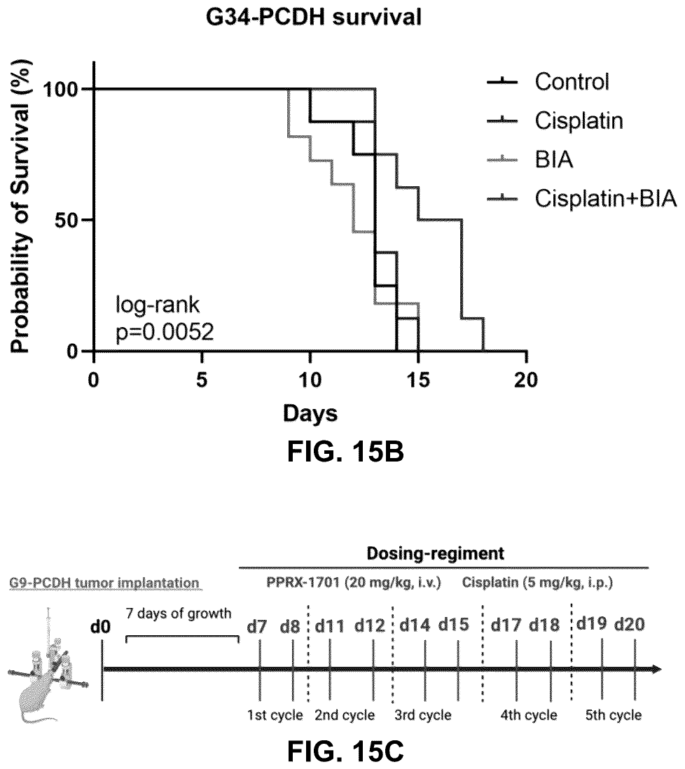

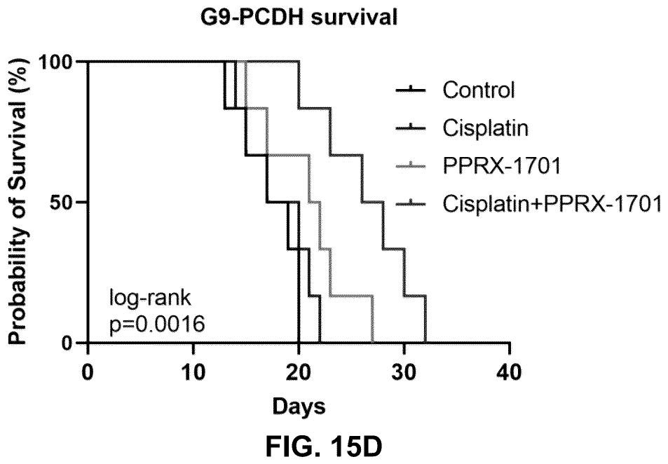

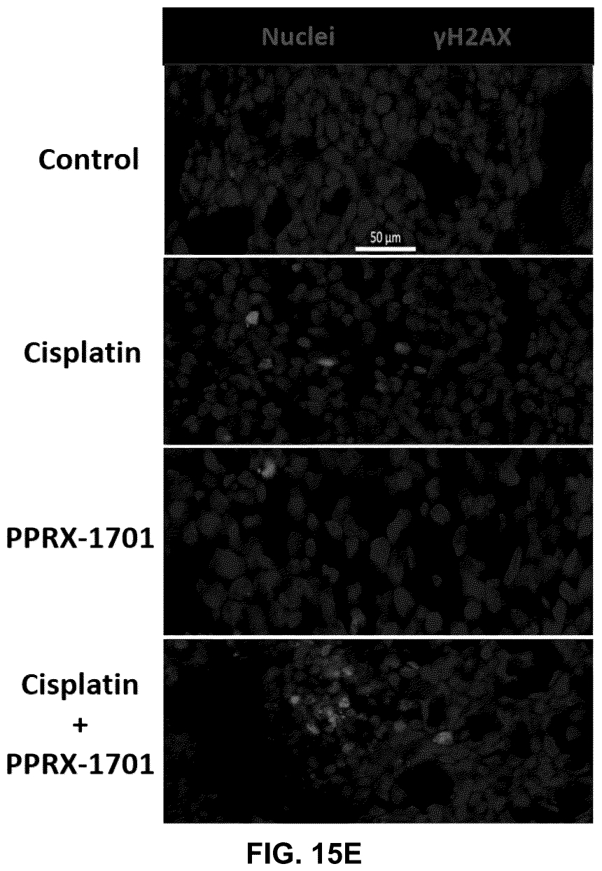

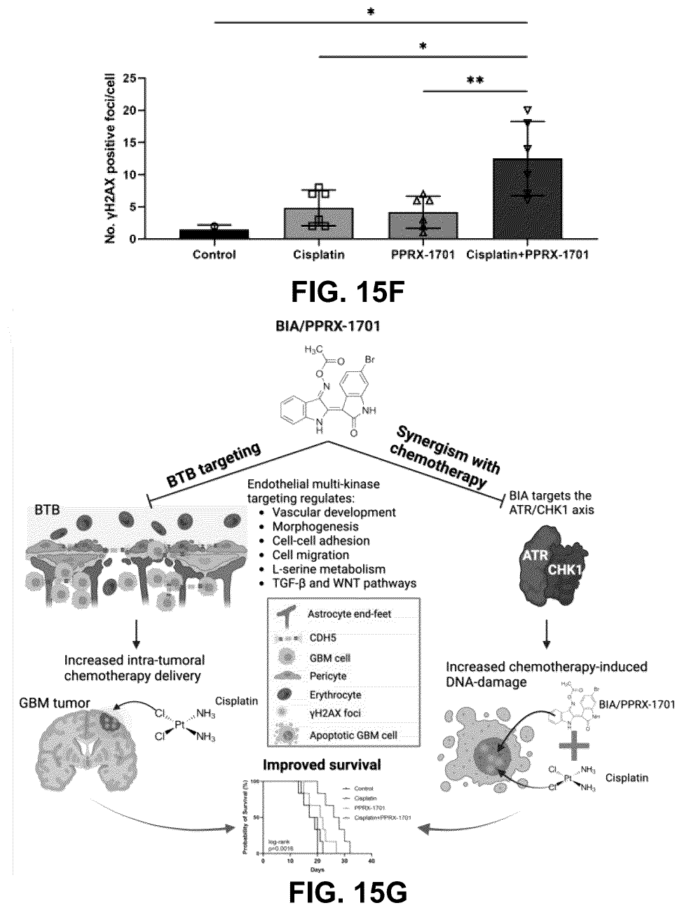

Administration of BIA prior to cisplatin treatment presents pre-clinical efficacy in patient derived xenografts of GBM: Finally, we looked to determine whether BIA and cisplatin combination regimens could provide a therapeutic effect in our intra-cranial GBM murine models. We proceeded with a dose-regime of BIA pre- and co-administration with cisplatin at 5 mg/kg to promote and maintain increased platinum delivery (FIG. 15A). The BIA and cisplatin combination regimens prolonged the survival of tumor-bearing mice significantly over the single-treatment and control arms, indicating efficacious results by this approach (FIG. 15B).

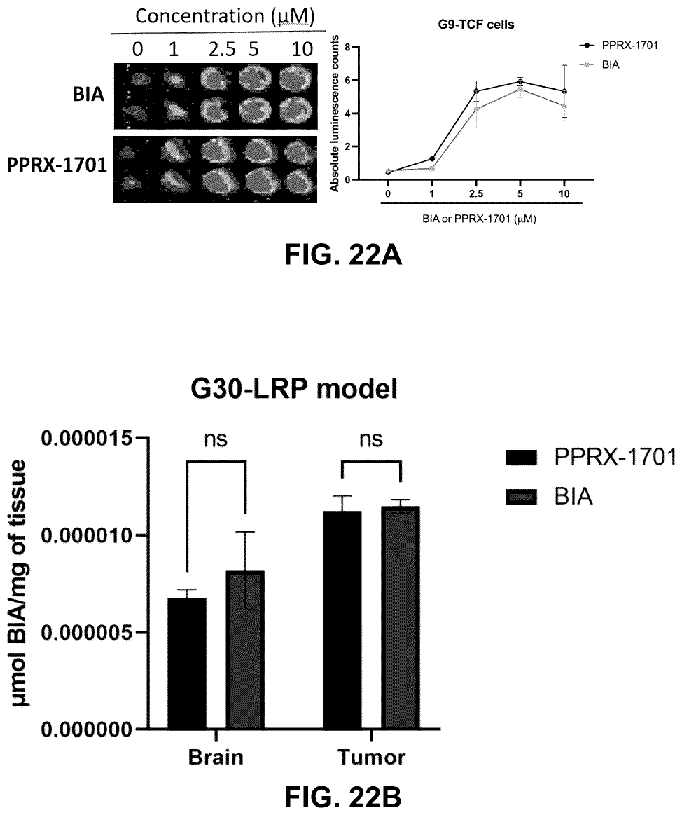

BIA is highly hydrophobic, making it difficult to dissolve in physiological solutions, which limits its clinical translation. To address this, we began a collaboration with Cytodigm, Inc., Natick, MA (previously Phosphorex, Inc., Hopkinton, MA), for generating suspendable BIA-loaded nanoparticles. These would enable intravenous BIA administration for systemic delivery and facilitate future toxicologic, pharmacodynamic and pharmacokinetic studies. The resulting BIA loaded nanoparticles, called PPRX-1701, have shown pre-clinical efficacy in murine GBM,⁴⁶ maintains GSK3-b inhibition capacity, as indicated by a G9-TCF cell line reporter (FIG. 22A). Moreover, biodistribution analysis of i.p. BIA or PPRX-1701 administration show similar levels of BIA localization in brain and GBM tumor tissues in mice (FIG. 22B), confirming that BIA retains kinase inhibition and biodistribution properties when loaded into the nanoparticles. We implanted a second patient-derived GBM xenograft model (FIG. 15C) and performed systemic pre-administrations of PPRX-1701 before cisplatin injections. Combination of PPRX-1701 with cisplatin were also more efficacious in comparison with empty nanoparticles plus cisplatin, PPRX-1701 alone and non-treated controls (FIG. 15D). Assessment of DNA damage by gH2AX staining indicated that PPRX-1701 enhanced the genotoxicity of cisplatin, correlating with the extended survival observed (FIG. 15E and FIG. 15F). Altogether, our data highlights potential molecular targets associated to the BTB in GBM. In addition, we demonstrated that BIA exerts pre-clinical efficacy in GBM murine models through its dual capacity to selectively target transcriptional programs of the BTB, promoting intra-tumoral drug delivery and by showing cytotoxic synergistic effects with DNA-damaging chemotherapy (FIG. 15G).

Systemic administration of BIA or PPRX-1701 in combination with cisplatin treatment shows enhanced pre-clinical efficacy in murine GBM models. (FIG. 15A) and (FIG. 15C) Diagrams of the experimental design for G34-PCDH and G9-PCDH xenograft efficacy studies using BIA/PPRX-1701 and cisplatin combinations. (FIG. 15B) Efficacy studies of G34-PCDH xenograa using BIA and (FIG. 15D) PPRX-1701 in combination with cisplatin. For PPRX-1701 studies, empty nanoparticles were used as controls and in combination with cisplatin. N=8/group. Log-rank test analysis for statistical significance. (FIG. 15E) Confocal immunofluorescence imaging of γH2AX (Alexa Fluor 647, red) nuclear foci from tumor tissue collected from study (D). Nuclei were stained with Hoechst 33342 (blue). Representative pictures taken at 20×. Scale bar=50 μm. (FIG. 15F) Quantification of γH2AX foci from (FIG. 15E) using Image J, n=6/group. Ordinary One-way ANOVA was performed for statistical evaluation. *p=0.01, **p=0,0086. (FIG. 15G) Schematic of proposed model of BIA/PPRX-1701 mechanism of action and its effects in GBM tumor drug delivery and anti-oncogenicity.

Discussion: Effective intra-tumoral drug delivery in brain cancer has undermined existing therapies to provide clinical opportunities for patients. Here, we have identified a network of genes associated with the BTB in GBM, and have demonstrated the dual functionality of the indirubin-derivative, BIA, to increase intra-tumoral drug delivery by targeting the BTB and enhance chemotherapy cytotoxicity via DNA-repair machinery modulation. Our work should provide grounds to establish further pre-clinical and, potentially, clinical studies for progressing BIA towards neuro-oncologic applications.

Following an in-silico strategy, we identified a set of 12 genes with elevated regional expression within the tumoral endothelium in GBM, suggesting a functional relevance for this disease. Most of these genes have been related to angiogenesis and blood-vessel recruitment, especially ACVRL1 (also called ALK1), CD93, ENG, FLT4 (VEGFR3) and PDGFRB. Previous studies⁵⁹ have indicated the co-expression of ACVRL1, CDH5, CLEC14A, PECAM1, ENG, GRP4, ROBO4 and PCDH12 in the tumor associated endothelium in several solid tumor types, including GBM. In fact, this gene set has been related to vascular development, blood vessel morphogenesis and tumor angiogenesis processes. These reports strongly correlate with our findings and support the functional relevance of these genes to the BTB in GBM. However, to the best of our knowledge, our work is first to link the modulation of these genes to BTB permeability for improved drug delivery in tumors. Future work by us would involve functional studies on these molecules for deeper understanding of their involvement in BTB biology.

Our screening led us to identify CDH5 (VE-cadherin) as a central element in the tumoral vasculature transcriptome. CDH5 is fundamental for endothelial barrier integrity, but its role in the BTB permeability is not fully understood. CDH5 showed a prominent expression in vascular hyperproliferative regions of GBM clinical samples and correlated its expression with endothelial markers above non-tumor cortex when analyzed by spatial transcriptomics. CDH5 downregulation strongly correlated with increased drug accumulation amer BIA injection in our GBM murine models. Functionally, CDH5 has been associated with enabling vascular mimicry capacity to GSCs,⁶⁰ permitting formation of vascular-like structures that supply with nutrients and facilitate anti-angiogenic therapy resistance. Our findings show that BIA targets the CDH5 gene expression in endothelial and tumoral compartments. It is possible that this simultaneous cellular targeting is, at least, partially responsible for the increased NaF and cisplatin accumulation we observed. On the other hand, we encountered altered expression levels of genes involved in TGFb and WNT pathways. The TGF-b pathway maintains BBB integrity through crosstalk with oligodendrocytes, and pericyte and endothelial cells.^{61,62} We observed downregulation of several members of this pathway, except for TCF7, that showed an elevated expression. The WNT/bcatenin pathway is fundamental for brain and retinal barrier genesis and maintenance, especially the Norrin/WNT7A/B axis.^{63,64} We observed a decrease of WNT7B, and WNT ligand receptors FZD4 and FZD7, with simultaneous increase of expression of WNT4, WNT10A and WNT11 ligands. Additionally, transcriptional alteration of genes involved in angiogenesis (i.e. ANGPT2, ENG, ANG) and BTB permeability (S1PR1 and S1PR3) was also seen. As such, CDH5 downregulation and alteration of BBB-integrity components might altogether contribute to the BTB permeability modulation exerted by BIA. Future work by our team will focus on functional interrogation of the potential roles of CDH5 in the tumor-associated vasculature and relevance in the permeability of the BTB for drug delivery purposes.

The administration of BIA to tumor-bearing xenograft and syngeneic mice enhanced the accumulation of cisplatin and NaF in brain tumor tissue but not healthy brain. The specificity of this effect towards tumorigenic regions remains under study by us. It is likely that BIA, being a small-molecule kinase inhibitor, targets cells undergoing elevated kinase signaling activity, such as the case of angiogenic/proliferative endothelium, but spares slow cycling/quiescent cells that constitute the non-tumorigenic brain vascular networks. Vascular development and morphogenesis programs are active in angiogenic endothelial cells, and the multi-targeting quality of BIA can dysregulate multiple arms of these programs. Our findings showed downregulation of ERG, a transcriptional master regulator of endothelial identity and a relevant marker for tumor-associated vasculature.⁶⁵⁻⁶⁹ We also observed decreased activity of the endothelial nitric oxide synthase (eNOS), important blood-pressure regulator, and the p38a/CREB axis, which can control gene expression of CDH5 and other genes important to endothelial biology. On the other hand, BIA promoted the expression of genes relevant to L-serine metabolism and amino-acid transport processes. L-serine has been reported to improve cerebral blood-flow, which provides neuroprotection during CNS disease.^{65,70} In this regard, a normalized blood-irrigation can also promote drug accumulation in solid tumors. The mTORC1 complex is an important amino-acid sensor, which regulates protein synthesis and energy modulation. We observed an increased phosphorylation of p70 S6 kinase, a downstream target of the mTORC1 pathway. This corroborates with the observation that BIA promotes amino-acid transport and synthesis, which might explain mTORC1 activation.

Simultaneous exposure to BIA and cisplatin had a synergistic killing effect in GSC-like cells. This correlated with increased DNA damage and ATR/CHK1 axis inhibition. Other studies have shown indirubin derivatives to induce DNA-damage in HCT-116 cancer cells.⁷¹ However, the present work reveals a novel applicability of BIA, and potentially other indirubins, in combinatorial regimens to synergize with DNA-damaging chemotherapy. Administration of BIA or PPRX-1701 nanoparticles, followed by cisplatin after 24 hours, permitted an extension of survival of two different GBM xenografts. Most likely this improved pre-clinical efficacy stems from the increased platinum delivery intratumorally and the additive cytotoxicity exerted by both agents. Given this finding, other DNA-damaging chemotherapeutics should be screened in combination with BIA to identify alternative drug candidates that would benefit from the increased accumulation and BIA anti-neoplastic synergism in GBM treatment. The mechanism of how BIA downregulates CHK1 expression at the protein level, and what alternative chemotherapy modalities will benefit with BIA remains in ongoing work by us.

Altogether, our work reveals novel molecular markers of the BTB, which in future studies should be functionally characterized to understand their role in the biology of the BTB-GBM interaction. The identification of BIA as a selective regulator of BTB permeability for improved drug delivery and potentiating agent of DNA-damaging chemotherapy supports the use of BIA in further preclinical and clinical studies of GBM. Primarily, further research should be pursued on screening for non-BBB penetrant chemotherapies and biologicals that would benefit from higher intratumoral internalization in combination with BIA, such as small molecule inhibitors, chemotherapies, and therapeutic antibodies.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Materials and Methods 1

Magnetic Resonance Imaging (MRI): MRI was performed at the BWH Imaging Core on a 7T Bruker MRI apparatus. G30 tumors were implanted (50,000 cells per mouse) intracranially into the striatum using a stereotactic frame. After three weeks of tumor growth, MRI images were collected according to BWH Imaging Core protocols using Gadolinium contrast enhancement. After the first image was collected, BiA was administered IP at 10 mg/kg), and imaging were performed again.

Electrical Impedance Measurements: Human Brain microvascular endothelial cell (purchased from Sciencell) monolayers were plated on special culture chambers on top of opposing circular gold electrodes (Applied Biophysics Inc). Impedance was automatically recorded during cell monolayer formation.

Inductively Coupled Plasma-Mass Spectrometry (ICP-MS): For platinum quantitation, ICP-MS was performed in an Agilent 7900 ICP mass spectrometer and controlled using the ICP-MS MassHunter software. Machine calibration was performed using a tuning solution (Agilent), containing 1 ng/mL (1 ppb) of Li, Co, Y, Ce and Ti trace-metals. In each experiment, the desired internal standard was added to each sample and standard solution. Standard curves were prepared with potassium tetrachloroplatinate (II) (Thermo Fisher) by generating dilutions of 0.1, 1, 10, 50, 100 and 1000 parts per billion (ppb). Isotopes selected for platinum detection were Pt¹⁹⁵. Performance reports were registered daily for calibration and accuracy records.

Survival studies: For survival studies, G9 tumors were implanted intracranially according to our established protocols. At day 7, animals were treated with 20 mg/kg BiA via tail vein, and on Day 8 with 5 mg/kg cisplatin via tail vein, with the cycle being repeated every three days. Animals were monitored every two days for weight loss and removed when predefined euthanasia endpoints were reached.

RNA Sequencing (RNAseq): RNA sequencing was performed by the Dana Faber molecular biology core according to their standard methods. RNA was prepared from human brain microvascular endothelial cell monolayers after 24 hours treatment with BiA. EXAMPLE 1 EFFECT OF INDIRUBIN ON THE BLOOD-TUMOR BARRIER (BTB)

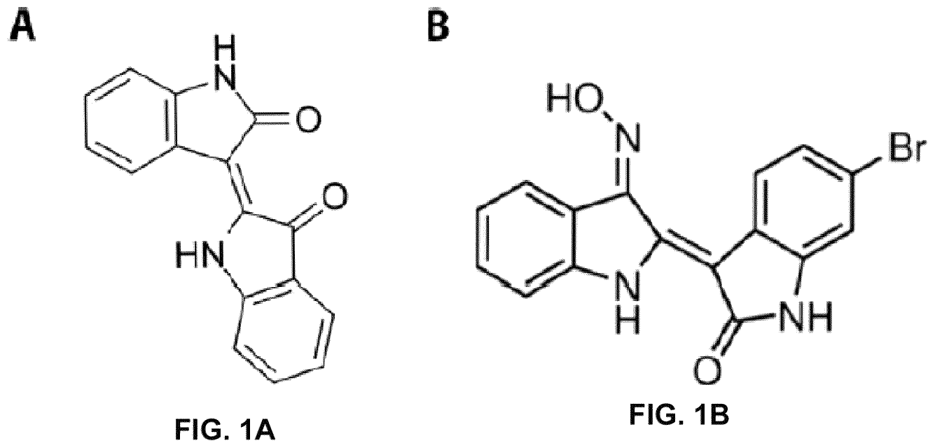

The present study investigated whether the indirubin derivative, BiA (see FIG. 1A and FIG. 1B), can disrupt the permeability of the BTB. Blood flow within a murine glioblastoma was assessed using dynamic contrast-enhanced magnetic resonance imaging (DCE-MRI) analysis and as measured by k^{trans}.

K^{trans} is calculated by measuring the accumulation of gadolinium-based contrast agent in the extravascular-extracellular space. Interpretation of k^{trans} varied according to permeability and blood flow. In situations where permeability is very low (near-intact blood brain barrier), then k^{trans} is a useful measure of permeability, whereas in situations where permeability is very high (disrupted blood brain barrier), then k^{trans} will reflect perfusion.

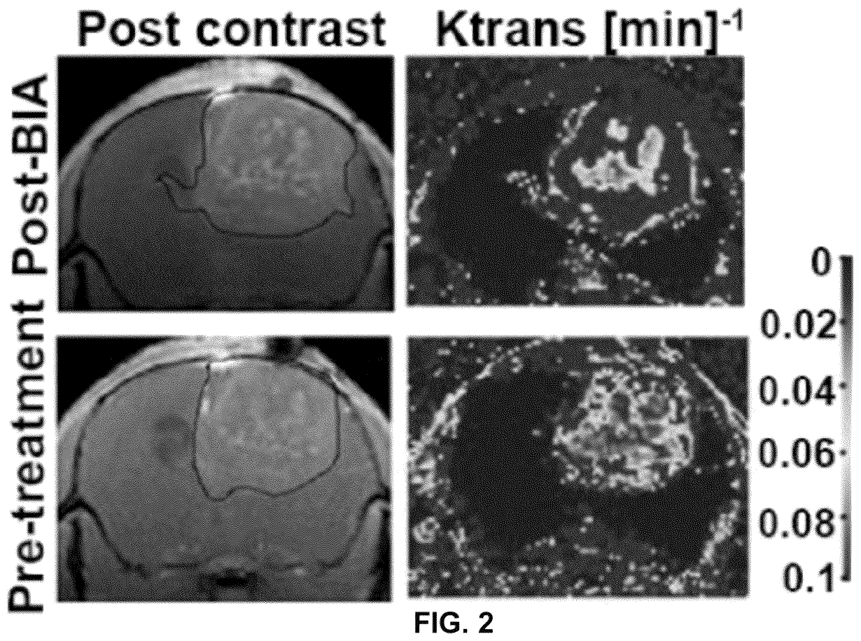

As shown in FIG. 2, k^{trans} values were significantly increased in the murine glioblastoma 24 hours after BiA treatment (FIG. 2, top right panel) when compared to pretreatment (FIG. 2, bottom right panel). Accordingly, the present data indicates that indirubin can effectively disrupt the blood-tumor barrier and increase vascular permeability to the brain tumor.

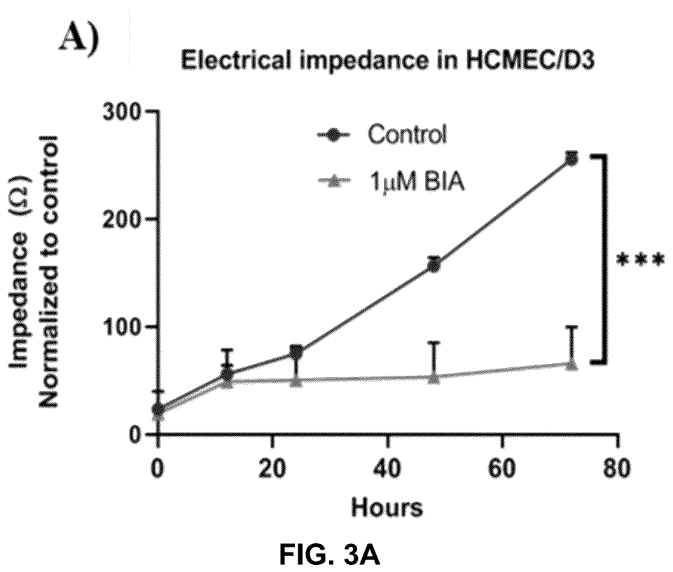

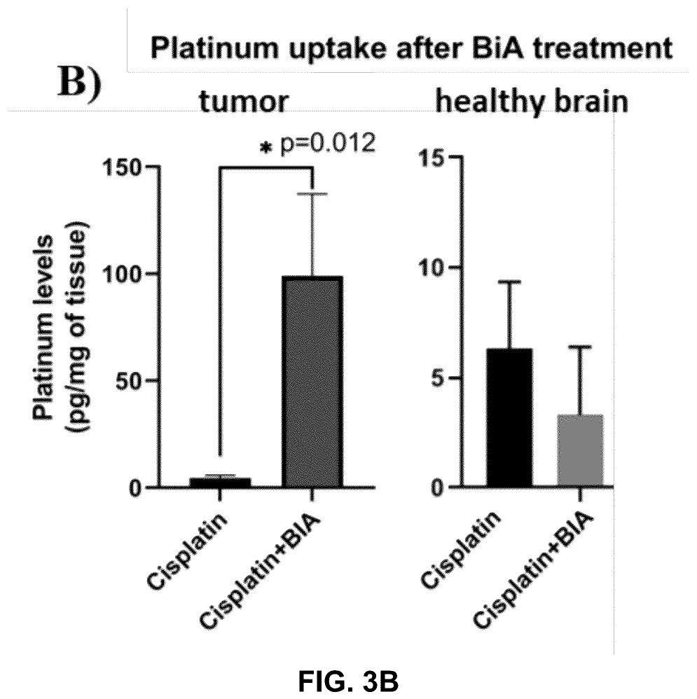

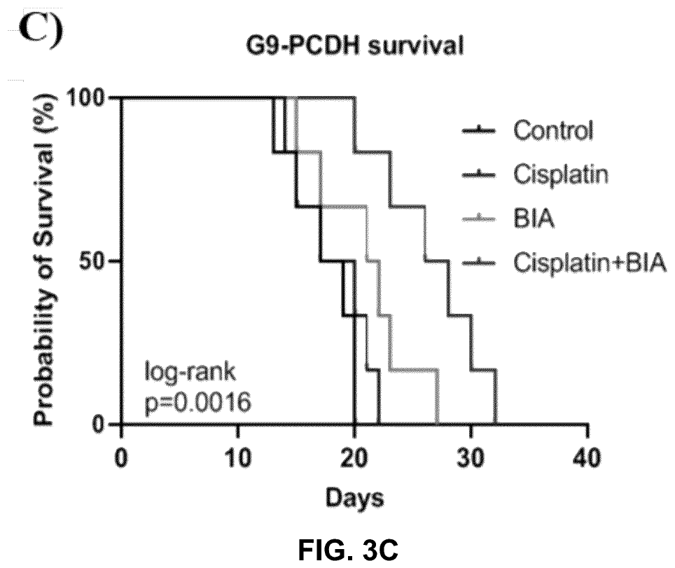

The in vitro effects of indirubin on endothelial monolayer permeability were also assess using Electric Cell-substrate Impedance Sensing (ECIS®) to provide a real-time, label-free, impedance-based study of cell behaviors in tissue culture. BiA (1 μM) was added to endothelial monolayers in an ECIS impedance measurement culture system (Applied Physics). As shown in FIG. 3A, BiA was shown to significantly decrease trans-endothelial electrical resistance of hCMEC/D3 brain microvascular endothelial cells (p<0.05). These data indicate that indirubin induces the loss of tight junction loss.

### Example 2 Effects of Indirubin Administration on Platinum Uptake in Healthy Brain and Brain with Glioblastoma Multiforme (GBM) Tumor and In Vivo Survival

The present study investigated whether the indirubin derivative, BiA, can increase the intratumoral accumulation of the anticancer drug, cisplatin, and whether such effects could extend the survival of animals afflicted with a GBM tumor.

For the assessment of the effects of indirubin on intratumoral accumulation of platinum, G9 tumors were implanted intracranially according to the protocols described above. Cisplatin (5 mg/kg) was injected via the tail vein 24 hours post-BIA injection (20 mg/kg vial tail vein), and accumulation measured after a further 5 hours. As shown in FIG. 3B, the administration of BiA significantly increases the in vivo uptake of platinum in tumor tissue of brain-tumor bearing mice, but not in healthy mouse brain.

For survival studies, G9 tumors were implanted intracranially as described above. At day 7, mice were treated with 20 mg/kg BiA via tail vein, and on Day 8 with 5 mg/kg cisplatin via tail vein, with the cycle being repeated every three days. As shown in FIG. 3C, BiA in combination with cisplatin increased survival in mice in GBM mouse xenografts. Single agent controls are also shown in FIG. 3C. Importantly, these data confirm that the increased in vivo uptake of the anticancer drug in the tumor significantly extends survival in the murine glioblastoma animal model.

### Example 3 Alterations to Gene Expression Profiles Induced by Indirubin

In order to assess changes in gene expression profiles induced by alterations to barrier functions induced by indirubin, RNA sequencing was performed RNA prepared from human brain microvascular endothelial cell monolayers after 24 hours treatment with BiA. HBMEC monolayers were treated with BiA (1 μM) for 24 hours and gene expression profiles compared with untreated controls.

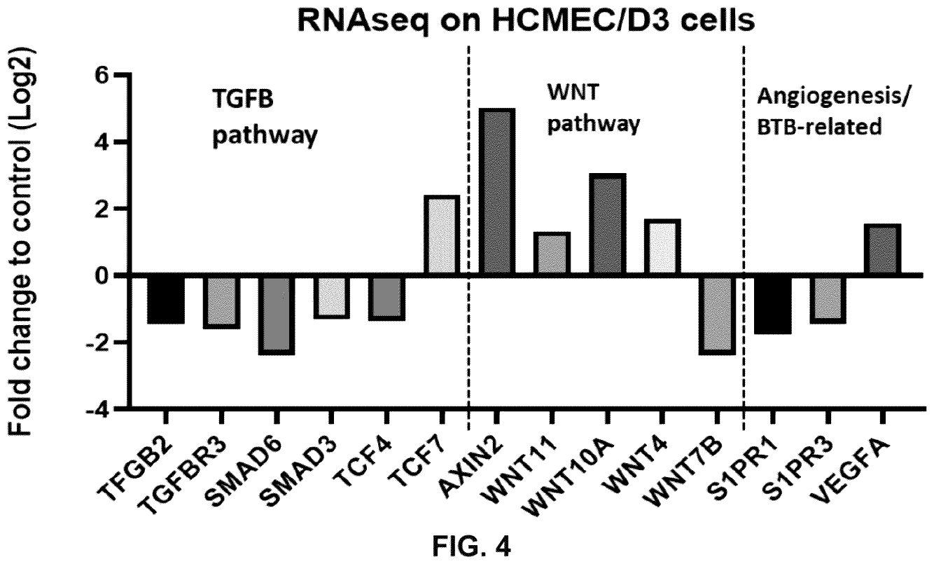

FIG. 4 shows the alteration to major pathways identified by gene ontology, including the Transforming Growth Factor β (TGF-β) pathway, the Wnt signaling pathway, and the angiogenesis/BTB pathway induced by BiA. Wnt signaling is known to be critical in the formation of the BBB.⁷² TGF-β is a pleiotropic growth factor which has been strongly linked to the promotion of tumor angiogenesis.⁷³ The BTB angiogenesis signature involving VEGF is well known and is a target of anti-angiogenic therapies such as avastin.

### Materials and Methods 2

BTB-gene in silico screening: To identify genes related to BTB function, we initiated an in silico-based approach by accessing bulk RNA-sequencing data from The Cancer Genome Atlas via the CBIO portal for Cancer Genomics (cbioportal.org). We initiated a correlation analysis of genes coexpressed with endothelial markers PECAM-1 (CD31), Von-Willebrand Factor (VWF), C-lectin 14 type A (CLEC14A) and CD34, previously identified as useful markers of GBM vasculature.⁴⁷ A selection of top-50 genes (Spearman's rank correlation) commonly observed in 3 out of the 4 markers was done and interrogated their expression levels in GBM tumors in comparison to healthy brain by using the GlioVIS portal (gliovis.bioinfo.cnio.es) to visualize the Rembrandt study.⁷⁴ Those genes significantly elevated in tumor over healthy brain were selected as candidate BTB targets due to their possible relevance in GBM. Then, their regional expression in GBM was assessed by using the IVY GAP (glioblastoma.alleninstitute.org) data visualized in the GlioVIS portal. Using this tool we confirmed their expression in microvascular proliferative regions, which are associated with the vasculature in tumors. All graphs of GlioVIS and cBIO portal datasets were generated in the corresponding websites and pairwise t-tests performed for statistical significance test.

Gene Ontology analysis: For Gene Ontology (GO) analyses, we used the EnrichR (maayanlab.cloud/Enrichr/) website, generated by the Ma′ayan's lab.⁷⁵⁻⁷⁷ We used the GO Biological Process 2023 visualization tool to identify biological processes of the identified gene-sets. The Appyters notebook⁷⁸ linked to EnrichR was used for graphics visualization.

Gene interaction network analysis and gene-set clustering: Gene-sets were submitted to the Search Tool for the Retrieval of Interacting Genes (STRING, /string-db.org/⁷⁹). Scores were set to medium interaction (0.4). For interaction analysis of the genes targeted by BIA, we selected genes upregulated and downregulated by BIA equals or above 2-fold change (Log 2). Only genes that presented interaction were associated by a 4-kmeans clustering. Gene-sets comprising each cluster were submitted to GO analysis (using EnrichR as mentioned above) and ranked by p-value significance. The most significant pathway by this method is indicated by color-code (or by different shading) in each cluster.

Spatial transcriptomics data set analysis: Using GBM sample spatial transcriptomics data available from Ravi et al., 2023,⁴⁸ we used SPATA2 (R-studio)⁸⁰ for spatial analysis of CDH5 gene expression in brain cortex and tumor regions. Spatial gene expression analysis was performed using SPATA and SPATA2 as indicated in Ravi, et al., 2023.⁴⁸

Mice: Female Nu/Nu mice (Envigo) and C57/BL6 (Charles River Laboratories) aged 8 weeks were used for in vivo experiments. All our procedures followed the guidelines by the Institutional Animal Care and Use Committee (IACUC) with support of the Center for Animal Resources and Education (CARE) at Brown University.

Cell lines: Glioma-stem cell-like cell lines G9-PCDH, G34-PCDH, G33-PCDH, G62-PCDH and G30-LRP were obtained and cultured as previously described.^{42,46,81} Briefly, cells were grown as neuro-spheres using Neurobasal medium (Gibco) supplemented with 20 ng/mL of human recombinant EGF (Peprotech), 20 ng/mL of human recombinant FGF (Peprotech), 2% B-27 supplement (Thermo Fisher Scientific), 0.1% GlutaMax (Thermo Fisher Scientific), and 0.1% penicillin/streptomycin (Thermo Fisher Scientific). Cells were left to grow at least overnight for sphere formation. For single-cell dissociation, Accutase (Gibco) was used for 5 minutes at 37 degrees. For culturing GL261-Luc2 cells, we used 10% fetal bovine serum (Gibco), with 0.1% GlutaMax and 0.1% penicillin/streptomycin in DMEM/F12 media (Gibco).

Growth and culturing of immortalized human cerebro-microvascular endothelial cells (HCMEC/D3) (Sigma), primary human brain microvascular endothelial cells (HBMEC) (ScienCell), human primary astrocytes (Lonza Biosciences) and human primary pericytes (ScienCell) was performed as previously reported.^{82,83} Briefly, HCMEC/D3 and HBMEC cells were cultured in Endothelial Cell Media (ScienCell) supplemented with fetal bovine serum, endothelial cell growth supplement and penicillin/streptomycin as provided by the company. Astrocyte and Pericyte cells were grown in complete formulations of astrocyte cell media (ScienCell) and pericyte cell media (ScienCell), respectively. For immunostaining experiments HCMEC/D3 and HBMEC cells were grown in type 1 rat collagen-coated plates. These endothelial cells were used below passage 20 for maintenance of their BBB properties.

Cell viability assay: Cells were plated at a density of 1500 cells/well in black-well clear bottom 96-well plates and left growing in culture conditions overnight. Next day, cells were treated with titrating doses of the indicated compounds. For BIA only cytotoxicity studies, cells were incubated with BIA for 96 hours. For BIA and cisplatin combinatorial studies, cells were incubated with BIA and cisplatin, and corresponding controls, for 5 days. Next, we used the Cell-Titer Glo 3D (Promega) following provider's guidelines and quantified for luminescence signal using a Molecular Devices SpectraMax M2 plate reader. Conditions were repeated in triplicates.

Growth in Low Attachment (GILA) assay: Fluorescently labelled GBM cells (G9-PCDH and G34-PCDH, GFP-labelled) were plated in clear ultra-low attachment 96-well plates (Costar) with a density of 2000 cells/well using 100 μl of complete Neurobasal medium. Then, cells were centrifuged at 1200 rpm for 3 minutes. Cells were treated as indicated above and fluorescence visualized using a Nikon Eclipse Ti2 microscope. Sphere diameter was measured using Image J software. Conditions were repeated in triplicates.

Synergy analysis of BIA and cisplatin combinations in GBM neuro-spheres in vitro: To identify if the BIA and cisplatin combinations present synergistic anti-neoplastic effects in GBM cell line neuro-spheres, we used the SynergyFinder 3.0 software.⁸⁴ For this, cell viability assays (see above) were performed. Concentrations of 0 μM, 0.3 μM, 1 μM and 3 μM were added in combination with 0 μM, 0.62 μM, 1.25 μM, 2.5 μM, 5 μM and 10 μM of cisplatin, accordingly, for an exposure duration of 5 days. Cell Titer Glo 3D assays were performed for cell viability assessment. SynergyFinder 3.0 analysis was done with LL4 curve fitting, with outlier correction, following a ZIP synergy score. We performed a ZIP-based analysis since this model low false positive rates while calculating synergy of anti-oncogenic drugs.⁸⁵ For reference, a d-score of less than −10, could signify antagonism, −10 to 10 could signify additivity, and above 10 could signify synergism.

RNA-sequencing of HCMEC/D3 cells treated with BIA: For RNA-sequencing, HCMEC/D3 cells were plated at a density of 500,000 cells/well in a 6-well plate. Left to grow for 24 hours, and then treated with 1 μM of BIA or DMSO (control). After 24 hours, cells were collected and processed for RNA extraction using the column-based RNeasy kit (QIAGEN), following provider's instructions. RNA quality and quantity were quantified using a Nanodrop™ One (Invitrogen). At least 500 ng of RNA was submitted for bulk RNA-sequencing at GeneWiz (Azenta Life Sciences). QC was accessed, and library was prepared with Poly(A) selection. Sequencing was performed using Illumina HiSeq.

Differential gene expression on the RNA-seq raw data (FASTQ files) was analyzed by Azenta Life Sciences using DESeq2 aligning to human transcriptome. Data QC was verified. Log₂ fold change (Log 2FC) was calculated by Log₂ (BIA group mean normalized counts/Control group mean normalized counts). The Wald test p-value and Benjamini-Hochberg adjusted p-value were calculated. A heatmap and volcano plot of top adjusted p-value differentially expressed genes (DEGs) in ensemble ID annotation bi-clustering to treatment conditions were generated. Control and BIA groups consisted of three-independent samples.

Immunofluorescent (IF) staining: For IF staining of HCMEC/D3 endothelial cells, we coated 8-well Nunc™ Lab-Tek™ chamber slides (Thermo Fisher Scientific) with 1× Type 1 rat-tail collagen (Corning) following provider's instructions. Then, we plated at a density of 50,000 cells/well and left in culture for 72 hours to allow for barrier formation. Next, we treated with BIA or control for 24-48 hours. Cells were then fixed with 10% formalin (Thermo Fisher Scientific) for 10 minutes, permeabilized for 30 minutes using 0.01% Triton X-100 and blocked with 0.1% normal donkey serum (Calbiochem) for 1 hour in 0.025% Tween-20 (Thermo Fisher Scientific) in Phosphate-Buffered Saline (PBS) (Gibco). Then, primary antibodies were added: mouse anti-CDH5 (VE-cadherin, BioLegend) 1:100, rabbit antiClaudin-5 (Thermo Fisher Scientific) 1:100, and mouse anti-ZO-1 (Invitrogen) 1:100, and incubated overnight in the cold. Next day, secondary antibodies were used for 2 hours at room temperature: Alexa Fluor 594 anti-mouse (1:500), Alexa Fluor 594 anti-rabbit (1:500), and Alexa Fluor 647 anti-mouse (1:500), all of these Thermo Fisher Scientific. For cytoskeleton staining Phalloidin-iFluor 488 (Abcam) 1:1000 for 30 minutes and nuclei staining using Hoechst 33342 (Thermo Fisher Scientific) 1:1000 for 5 minutes, at room temperature.

For GBM cell staining, cells were cultured in 10% DMSO in complete Neurobasal media for 2 days, and then plated at a density of 50,000 cells/well in 8-well Nunc™ Lab-Tek™ chamber slides. IF staining was performed as indicated above for endothelial cells. Primary antibodies used: Rabbit anti-gH2AX (Ser139) (Cell Signaling) at 1:100 dilution. A goat anti-rabbit Alexa Fluor 647 was used at 1:500.

For mouse brain tissue staining, brains were collected from CO₂ euthanized and PBS perfused tumor-bearing mice, and fixed in 10% formalin for 72 hours on rotation in the cold. Then, brains were transferred to 30% sucrose for 3 days at 4 degrees Celsius under rotation. Before cryo-sectioning, brains were frozen at −80 degree Celsius for more than 30 minutes, embedded in Optimal Cutting Temperature (OCT) compound (Fisher) and transferred to −27 degrees Celsius to a cryostat (Leica CM1950) for sectioning (20 μm thickness). Sections were placed on slides and staining followed as indicated above. All pictures were taken using a LSM 880 Zeiss confocal microscope.

BBB spheroids and dextran permeability assay: BBB spheroids were grown and cultured with a FITC-conjugated (70 kDa) fluorescent dextran (Millipore Sigma) as previously reported.^{51,86,87} BBB spheroids were grown for 48 hours and then treated with BIA at increasing doses for 72 hours. Then, spheroids were collected and stained as indicated above for CDH5 and F-actin (Phalloidin). In the case of fluorescent dextran incubation, BBB spheroids were collected in 1.5 mL microtubes (Eppendorf) and incubated for 3 hours at 37 degrees Celsius. Pictures were taken by confocal microscopy. For dextran permeability measurement, 21 images using Z-stack layers of 5 μm intervals for achieving a total depth of 100 μm within the sphere. Fluorescent-dextran intensity from maximal intensity projection was quantified using Image J (National Institute of Health).

Trans-endothelial electrical resistance: HCMEC/D3 or HBMEC cells were plated in 8W10E+ PET 8-well arrays (Applied Biophysics) at a density of 100,000 cells/well in 500 μl. These arrays were placed in a pre-stabilized ECIS Z-Theta instrument (Applied Biophysics). Using the ECIS Z-Theta software (Applied Biophysics), measurements were set to 4000 and 64000 Hz every 30 minutes. Cells were left to grow and form a barrier for 48-72 hours (normally, a resistance plateau would be reached, and capacitance showed at ˜10 nF for 64000 Hz). Cells would then be treated with BIA and left to grow up to 5 days, with frequent drug-containing media re-addition for maintenance of the culture. Resistance (W) and capacitance (nF) were recorded and plotted.

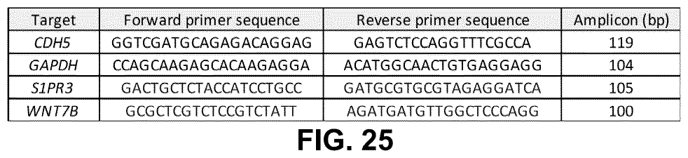

Real-time PCR: Total RNA from GBM and HCMEC/D3 cells was obtained and processed as indicated above. For cDNA generation, we used 1 μg of RNA and processed with with the iScript™ cDNA synthesis kit (BioRad), following the protocol indicated by the provider. All primers were designed using NCBI Primer-Blast tool. Detailed information on primer sequences can be found in FIG. 25 (Supplementary Table 3). Gene expression levels were quantified using PowerUp SYBR Green Master Mix (Applied Biosciences) on QuantStudio 6 Pro System (Applied Biosciences), normalized by housekeeping gene GAPDH expression and represented as relative expression using comparative ΔΔC_{T} method.

Western blot: HCMEC/D3 and GBM cell lysates were collected in RIPA Buffer (Thermo Fisher Scientific) supplemented with 1× protease/phosphatase inhibitor cocktail (Cell Signaling). Lysate collection from murine tumor tissue samples (˜30 mg) was performed under homogenization using 23G and 26G needles. Total protein concentration was measured using Pierce 660 nm Protein Assay Reagent (Thermo Fisher Scientific) at 660 nm absorbance in Molecular Devices SpectraMax M2 plate reader. Samples were incubated in 1× Laemmli sample buffer (BioRad) at 95 degrees Celsius for 5 minutes before loading onto 10% Mini-PROTEAN TGX precast protein gel (BioRad). PageRuler Plus Pre-stained Protein Ladder (Thermo Fisher Scientific) was used as ladder. Appropriate secondary antibodies, Goat anti-mouse-HRP (Sigma) or Goat anti-rabbit-HRP (Sigma) in 5% milk in 1×TBST with 1:5000 dilution.

Phospho-kinase array: HCMEC/D3 cells were plated at a density of 1 million cells and treated with either 1 μM BIA or vehicle DMSO for 24 hr. Cell lysates were collected with manufacturer provided Lysis Buffer 6 supplemented with 10 μg/mL Aprotinin (Tocris), 10 μg/mL Leupeptin hemisulfate (Tocris) and 10 μg/mL Pepstatin A (Tocris) for protein preservation. 50 μg of lysate from each sample were loaded into each membrane. All experiment procedures were performed using the Proteome Profiler Human Phospho-Kinase Array Kit (R&D Systems) following manufacturer's protocol.

siRNA transfections: G9-PCDH and G30 cells were cultured to approximately 60% confluency and transfected using Lipofectamine RNAiMax Transfection Reagent (Invitrogen) for 1 day, and then replated for western blot or cell viability assays. All experimental steps followed manufacturer's protocol. siCHK1 (Ambion) was used for CHK1 depletion. MISSION siRNA universal negative control (Sigma Aldrich) was used as control siRNA.

Flow cytometry for cell cycle, DNA damage and apoptosis assays: For cell cycle analysis, 100,000 HCMEC/D3 cells were plated in 6-well plates and treated with indicated concentrations of BIA or control for 48 hours. Then, cells were washed twice with PBS and fixed/permeabilized with 5 mL of cold 70% ethanol added dropped-wise while vortexing at low speed. Cells were stored for 1 day at −20 degrees Celsius, washed three times with PBS and treated with 20 μg/mL RNAse I (Thermo Fisher Scientific) and stained with anti-Ki-67 FITC-conjugated (1:1000) (BD Biosystems) and 1.5 μM propidium iodide (Thermo Fisher Scientific). Amer 30 minutes of incubation in the dark, cells were analyzed using a CytoFLEX system (Beckman Coulter). 50,000 events were counted, and data was analyzed using the CytoFLEX system software (Beckman Coulter).

For apoptosis assessment, HCMEC/D3 cells were treated as indicated above for 72 hours with BIA at indicated doses. Cells were collected from the 6-well plates and washed three times with PBS. Then, incubated with SYTOX™ Blue nucleic acid stain (5 mM) with a dilution of 1:1000 for 15 minutes. Cells were submitted and analyzed in the CytoFLEX system and its software as indicated above.

For DNA damage and cell cycle assessment of BIA and cisplatin, G62 cells were plated at a density of 100,000 cells/well in a 6-well plate and grown in complete Neurobasal media. Cells then were treated with BIA and/or cisplatin and control for 72 hours. Next, cells were collected and washed three times with PBS and stained with 1:500 of FITC anti-gH2AX Phospho (Ser139) (BioLegend) antibody and Propidium iodide (1 mg/ml) at 1:1000 dilution for 30 minutes in the dark. Cells were taken for analysis in a BD Fortessa cytometer and data analyzed using a FloJo software (BD Biosciences).

Secretome quantification: For cytokine analysis of brain endothelial cells after BIA exposure, HCMEC/D3 cells were plated at a density of 500,000 cells/well in a 6-well plate. Cells were treated with indicated doses of BIA or DMSO for 48 hours. Then, 1 mL of media was collected and processed for cytokine quantification in a Luminex platform (Thermo Fischer Scientific) following the provider's instructions.

BIA and PPRX-1701 preparation: BIA powder stocks (Millipore Sigma) were resuspended in DMSO at a concentration of 10 mM (in vitro usage) or 100 mM (in vivo usage). For animal experiments, 100 mM BIA was dissolved in 2% Tween-20 (Thermo Fisher Scientific), 1% polyethylene glycol (PEG) 400 (Thermo Fischer Scientific) in sterile PBS to achieve a concentration of 10 mM BIA. PPRX-1701 was prepared and generously provided by Cytodigm, Inc. as previously reported.⁴⁶

G9-TCF reporter assay: G9-TCF cells were engineered by over-expressing a luciferase gene (Luc2) controlled by a TCF7 recognized promoter in the G9-PCDH cell line. Cells were plated in 96-well dark-well clear flat bottom plates at a density of 1500 cells/well. Next day, cells were treated with increasing BIA doses for 5 hours and then exposed to 10 μg/mL of D-Luciferin (Goldbio). Luminescence signal was quantified in the IVIS system.

BIA quantification in vivo: G30-LRP cells were implanted in nude mice as previously indicated, left to grow for 14 days, and injected with 20 mg/kg of BIA or PPRX-1701 (intraperitoneal). After 1 hour in circulation, mice were euthanized, perfused and tumor and brain tissue were harvested. Tissue was frozen at −80 degrees Celsius until processing. Quantification of BIA was performed using a Q-Exactive HFX Orbitrap mass spectrometer (LC-HRMS) (Thermo Fisher Scientific). Sample processing and analysis was performed as previously described.⁴⁶

In vivo studies: For intra-cranial tumor implantation, GBM neuro-spheres were grown to 70% confluency before dissociated into single cell on the day of surgery. 50,000 cells were resuspended in 3 μl of sterile PBS and injected intracranially into the striatum (2 mm right hemisphere, 1 mm frontal, 3 mm depth from bregma) of mice under anesthesia and stereo-tactically fixed. Tumors were left to grow for approximately 2-3 weeks, depending the cell line. Animals were randomized to treatment groups. BIA injections consisted of 20 mg/kg (i.p.), except if indicated otherwise. PPRX-1701 was administered at 20 mg/kg (i.v.) via lateral tail-vein. Cisplatin injections were performed at 5 mg/kg (maximum tolerated dose, i.p.). All GBM tumor murine studies involved continuous condition and weight assessments, with endpoint considered when 20% of weight loss and/or moderate-to-high grimace scale and neurological symptoms were observed.

ICP-MS for platinum quantification: Mice treated with cisplatin after BIA administration were euthanized, intra-cardially perfused with PBS and tissue harvested to be stored at −80 degrees Celsius. Tissue was processed and platinum (Pt195) was quantified using an Agilent 7900 ICP-MS, as previously described.⁴²

Sodium fluorescein (NaF) BTB permeability studies: NaF was administered to G30 tumor-bearing mice intravenously (i.v.) via lateral tail vein at 20 mg/kg. Then, 30 minutes after administration when peak fluorescence is reached in the brain, mice were euthanized for immediate brain tissue harvest. Fresh brain samples were visualized in Xenogen in vivo imaging system (IVIS). Quantification of pixel intensities from acquired images was performed in ImageJ. Tissue samples were then homogenized in 1 mL of 60% trichloroacetic acid in PBS and measured at 488 nm in a Molecular Devices SpectraMax M2 plate reader.

Data and statistical analysis: Numerical results were analyzed, graphed, and statistically analyzed using the Prism software (GraphPad). Experiments were independently replicated at least three-times, unless indicated differently in the figure legends. Diagrams and workflow figures were generated using the BioRender software.

### CONCLUSIONS

The results of the above studies support the role of indirubin, or derivatives thereof, enhancing the uptake of a chemotherapeutic agent into a brain tumor and their use for the treatment of subjects afflicted with a malignant brain tumor.

### REFERENCES

^{1.} (2022) Cancer.Net Editorial Board. Brain Tumor: Statistics. https://www.cancer.net/cancer-types/brain-tumor/statistics.
^{2.} Isselbacher Kurt J., et al. Harrison's Principles of Internal Medicine, 13th Edition. Shock. 1996; 5(1):78.
^{3.} Porter R. S., & Kaplan, J. L. (Eds.). The Merck manual of diagnosis and therapy (19th ed.). Merck Sharp & Dohme Corp. 2011, (978-0-911910-19-3).
^{4.} Robert S. Porter et al., (eds.). The Encyclopedia of Molecular Cell Biology and Molecular Medicine. Blackwell Science Ltd.; 1999-2012, (9783527600908).
^{5.} Robert A. Meyers (ed.). Molecular Biology and Biotechnology: A Comprehensive Desk Reference. VCH Publishers, Inc.; 1995, (1-56081-569-8).
^{6.} Luttmann Werner. Immunology. Elsevier; 2006, (9780120885442).
^{7.} Kenneth Murphy Allan Mowat, Casey Weaver (eds.). Janeway's Immunobiology. Taylor & Francis Limited; 2014, (9780815345305).
^{8.} Krebs Jocelyn E., et al. Lewin's genes XI. 11th ed. Burlington, Mass.: Jones & Bartlett Learning; 2014, (1449659055).
^{9.} Green Michael R. Molecular cloning: a laboratory manual/Michael R. Green, Joseph Sambrook. Cold Spring Harbor, N.Y: Cold Spring Harbor Laboratory Press; 2012, (1936113414).
^{10.} Davis et al. Basic Methods in Molecular Biology. Elsevier Science Publishing, Inc.; 2012, (044460149X).
^{11.} Lorsch J. Methods in Enzymology. Laboratory methods in enzymology: DNA. Preface. Methods Enzymol. 2013; 529:xix.
^{12.} Frederick M. Ausubel (ed.). Current Protocols in Molecular Biology (CPMB). John Wiley and Sons 2014, (9780471503385).
^{13.} John E. Coligan (ed.). Current Protocols in Protein Science (CPPS). John Wiley and Sons, Inc.; 2005, (19343655, 19343663).
^{14.} John E. Coligan A D A M Kruisbeek, David H Margulies, Ethan M Shevach, Warren Strobe, (eds.) Current Protocols in Immunology (CPI). John Wiley and Sons, Inc.; 2003, (9780471142737).
^{15.} Louis D. N., et al. The 2016 World Health Organization Classification of Tumors of the Central Nervous System: a summary. Acta Neuropathol. 2016; 131(6):803-820.
^{16.} Levin V. A., Leibel, S. A., Gutin, P. H. Neoplasms of the central nervous system. In: Devita S. Hellman, S., Rosenberg, S. A., ed. CANCER: PRINCIPLES AND PRACTICE OF ONCOLOGY. 3rd Ed. Philadelphia, PA: Lippincott; 1989:1557-1611.
^{17.} Kleihues P., Ohgaki H. Primary and secondary glioblastomas: from concept to clinical diagnosis. Neuro Oncol. 1999; 1(1):44-51.
^{18.} Schoenberg B. S. Epidemiology of central nervous system tumor. In: Walker M D, ed. ONCOLOGY OF THE NERVOUS SYSTEM. Boston: Nijhoff; 1983:1-30.
^{19.} Qi-yue Yang, et al. From natural dye to herbal medicine: a systematic review of chemical constituents, pharmacological effects and clinical applications of indigo naturalis. Chinese Medicine. 2020; 15(1):127.
^{20.} Gaboriaud-Kolar N., et al. Indirubin derivatives: a patent review (2010—present). Expert Opin Ther Pat. 2015; 25(5):583-593.
^{21.} U.S. Pat. No. 10,435,367: “Indirubin derivatives, and uses thereof.” Issued Oct. 8, 2019.
^{22.} Shriver J. A., et al. A tunable synthesis of indigoids: targeting indirubin through temperature. RSC Adv. 2022; 12(9):5407-5414.
^{23.} U.S. Pat. No. 10,039,829 “Nanoparticles of indirubin, derivatives thereof and methods of making and using same.” Issued Aug. 7, 2018.
^{24.} Blažević T., et al. Indirubin and Indirubin Derivatives for Counteracting Proliferative Diseases. Evid Based Complement Alternat Med. 2015; 2015:654098.
^{25.} U.S. Pat. No. 9,051,306 “Indirubin derivative having highly selective cytotoxicity for malignant tumors.” Issued Jun. 9, 2015.
^{26.} International Patent Application WO 0137819 A3 “Use of indirubine derivatives for making medicines.” Published May 31, 2001.
^{27.} Yang L., et al. Pharmacological properties of indirubin and its derivatives. Biomed Pharmacother. 2022; 151:113112.
^{28.} McDannold N., et al. Acoustic feedback enables safe and reliable carboplatin delivery across the blood-brain barrier with a clinical focused ultrasound system and improves survival in a rat glioma model. Theranostics. 2019; 9(21):6284-6299.
^{29.} Dréan A., et al. Temporary blood-brain barrier disruption by low intensity pulsed ultrasound increases carboplatin delivery and efficacy in preclinical models of glioblastoma. J Neurooncol. 2019; 144(1):33-41.
^{30.} Nessler I., et al. Increased Tumor Penetration of Single-Domain Antibody-Drug Conjugates Improves In Vivo Efficacy in Prostate Cancer Models. Cancer Res. 2020; 80(6):1268-1278.
^{31.} Ostrom Q. T., et al. CBTRUS Statistical Report: Primary Brain and Other Central Nervous System Tumors Diagnosed in the United States in 2012-2016. Neuro Oncol. 2019; 21(Suppl 5):v1-v100.
^{32.} Grochans S., et al. Epidemiology of Glioblastoma Multiforme-Literature Review. Cancers (Basel). 2022; 14(10).
^{33.} Sweeney M. D., et al. Blood-Brain Barrier: From Physiology to Disease and Back. Physiol Rev. 2019; 99(1):21-78.
^{34.} Steeg P. S. The blood-tumour barrier in cancer biology and therapy. Nat Rev Clin Oncol. 2021; 18(11):696-714.
^{35.} Arvanitis C. D., et al. The blood-brain barrier and blood-tumour barrier in brain tumours and metastases. Nat Rev Cancer. 2020; 20(1):26-41.
^{36.} Lockman P. R., et al. Heterogeneous blood-tumor barrier permeability determines drug efficacy in experimental brain metastases of breast cancer. Clin Cancer Res. 2010; 16(23):5664-5678.
^{37.} Arvanitis C. D., et al. Mechanisms of enhanced drug delivery in brain metastases with focused ultrasound-induced blood-tumor barrier disruption. Proc Natl Acad Sci USA. 2018; 115(37):E8717-e8726.
^{38.} Bunevicius A., et al. Focused Ultrasound Strategies for Brain Tumor Therapy. Oper Neurosurg (Hagerstown). 2020; 19(1):9-18.
^{39.} Sperring C. P., et al. Convection-enhanced delivery of immunomodulatory therapy for high-grade glioma. Neurooncol Adv. 2023; 5(1):vdad044.
^{40.} Cai Q., et al. Optical blood-brain-tumor barrier modulation expands therapeutic options for glioblastoma treatment. Nat Commun. 2023; 14(1):4934.
^{41.} Gregory J. V., et aL. Systemic brain tumor delivery of synthetic protein nanoparticles for glioblastoma therapy. Nat Commun. 2020; 11(1):5687.
^{42.} Jimenez-Macias J. L., et al. A Pt(IV)-conjugated brain penetrant macrocyclic peptide shows pre-clinical efficacy in glioblastoma. J Control Release. 2022; 352:623-636.
^{43.} Meijer L., et al. GSK-3-selective inhibitors derived from Tyrian purple indirubins. Chem Biol. 2003; 10(12):1255-1266.
^{44.} Schepetkin I. A., et al. Oximes: Novel Therapeutics with Anticancer and Anti-Inflammatory Potential. Biomolecules. 2021; 11(6).
^{45.} Williams Shante P., et al. Indirubins Decrease Glioma Invasion by Blocking Migratory Phenotypes in Both the Tumor and Stromal Endothelial Cell Compartments. Cancer Research. 2011; 71(16):5374-5380.
^{46.} Zdioruk M., et al. PPRX-1701, a nanoparticle formulation of 6′-bromoindirubin acetoxime, improves delivery and shows efficacy in preclinical GBM models. Cell Rep Med. 2023; 4(5):101019.
^{47.} Dusart P., et al. A Systems-Based Map of Human Brain Cell-Type Enriched Genes and Malignancy-Associated Endothelial Changes. Cell Rep. 2019; 29(6):1690-1706.e1694.
^{48.} Ravi V. M., et al. Spatially resolved multi-omics deciphers bidirectional tumor-host interdependence in glioblastoma. Cancer Cell. 2022; 40(6):639-655.e613.
^{49.} Phoenix T. N., et al. Medulloblastoma Genotype Dictates Blood Brain Barrier Phenotype. Cancer Cell. 2016; 29(4):508-522.
^{50.} Griveau Amelie, et al. A Glial Signature and Wnt7 Signaling Regulate Glioma-Vascular Interactions and Tumor Microenvironment. Cancer Cell. 2018; 33(5):874-889.e877.
^{51.} Cho C. F., et al. Blood-brain-barrier spheroids as an in vitro screening platform for brain-penetrating agents. Nat Commun. 2017; 8:15623.
^{52.} Kanki H., et al. CREB Coactivator CRTC2 Plays a Crucial Role in Endothelial Function. J Neurosci. 2020; 40(49):9533-9546.
^{53.} Mayo L. D., et al. Vascular endothelial cell growth factor activates CRE-binding protein by signaling through the KDR receptor tyrosine kinase. J Biol Chem. 2001; 276(27):25184-25189.
^{54.} Gustin J. A., et al. Tumor necrosis factor activates CRE-binding protein through a p38 MAPK/MSK1 signaling pathway in endothelial cells. Am J Physiol Cell Physiol. 2004; 286(3):C547-555.
^{55.} Xi X., et al. ACT001, a novel PAl-1 inhibitor, exerts synergistic effects in combination with cisplatin by inhibiting PI3K/AKT pathway in glioma. Cell Death Dis. 2019; 10(10):757.
^{56.} Wu M., et al. A PLK1 kinase inhibitor enhances the chemosensitivity of cisplatin by inducing pyroptosis in oesophageal squamous cell carcinoma. EBioMedicine. 2019; 41:244-255.
^{57.} Lopergolo A., et al. Synergistic cooperation between sunitinib and cisplatin promotes apoptotic cell death in human medullary thyroid cancer. J Clin Endocrinol Metab. 2014; 99(2):498-509.
^{58.} Qiu Z., et al. ATR/CHK1 inhibitors and cancer therapy. Radiother Oncol. 2018; 126(3):450-464.
^{59.} Bocci M., et al. Activin receptor-like kinase 1 is associated with immune cell infiltration and regulates CLEC14A transcription in cancer. Angiogenesis. 2019; 22(1):117-131.
^{60.} Mao X. G., et al. CDH5 is specifically activated in glioblastoma stemlike cells and contributes to vasculogenic mimicry induced by hypoxia. Neuro Oncol. 2013; 15(7):865-879.
^{61.} Seo Ji Hae, et al. Oligodendrocyte Precursor Cells Support Blood-Brain Barrier Integrity via TGF-β Signaling. PLOS ONE. 2014; 9(7):e103174.
^{62.} Schumacher L., et al. TGF-Beta Modulates the Integrity of the Blood Brain Barrier In Vitro, and Is Associated with Metabolic Alterations in Pericytes. Biomedicines. 2023; 11(1).
^{63.} Wang Y., et al. Interplay of the Norrin and Wnt7a/Wnt7b signaling systems in blood-brain barrier and blood-retina barrier development and maintenance. Proc Natl Acad Sci USA. 2018; 115(50):E11827-e11836.
^{64.} Chang J., et al. Gpr124 is essential for blood-brain barrier integrity in central nervous system disease. Nat Med. 2017; 23(4):450-460.
^{65.} Kalna V., et al. The Transcription Factor ERG Regulates Super-Enhancers Associated With an Endothelial-Specific Gene Expression Program. Circ Res. 2019; 124(9):1337-1349.
^{66.} Birdsey Graeme M., et al. Transcription factor Erg regulates angiogenesis and endothelial apoptosis through VE-cadherin. Blood. 2008; 111(7):3498-3506.
^{67.} Birdsey G. M., et al. The endothelial transcription factor ERG promotes vascular stability and growth through Wnt/β-catenin signaling. Dev Cell. 2015; 32(1):82-96.
^{68.} Haber M. A., et al. ERG is a novel and reliable marker for endothelial cells in central nervous system tumors. Clin Neuropathol. 2015; 34(3):117-127.
^{69.} Dryden N. H., et al. The transcription factor Erg controls endothelial cell quiescence by repressing activity of nuclear factor (NF)-κB p65. J Biol Chem. 2012; 287(15):12331-12342.
^{70.} Kalna V., et al. The Transcription Factor ERG Regulates Super-Enhancers Associated With an Endothelial-Specific Gene Expression Program. Circ Res. 2019; 124(9):1337-1349.
^{71.} Wan S., et al. Indirubin derivatives as bifunctional molecules inducing DNA damage and targeting PARP for the treatment of cancer. Eur Med Chem. 2023; 261:115843.
^{72.} Kalluri R. Basement membranes: structure, assembly and role in tumour angiogenesis. Nat Rev Cancer. 2003; 3(6):422-433.
^{73.} Batlle Raquel, et al. Regulation of tumor angiogenesis and mesenchymal-endothelial transition by p38a through TGF-β and JNK signaling. Nature Communications. 2019; 10(1):3071.
^{74.} Gusev Y., et al. The REMBRANDT study, a large collection of genomic data from brain cancer patients. Sci Data. 2018; 5:180158.
^{75.} Xie Z., et al. Gene Set Knowledge Discovery with Enrichr. Curr Protoc. 2021; 1(3):e90.
^{76.} Kuleshov M. V., et al. Enrichr: a comprehensive gene set enrichment analysis web server 2016 update. Nucleic Acids Res. 2016; 44(W1):W90-97.
^{77.} Chen Edward Y., et al. Enrichr: interactive and collaborative HTML5 gene list enrichment analysis tool. BMC Bioinformatics. 2013; 14(1):128.
^{78.} Clarke D. J. B., et al. Appyters: Turning Jupyter Notebooks into data-driven web apps. Patterns (N Y). 2021; 2(3):100213.
^{79.} Szklarczyk D., et al. The STRING database in 2023: protein-protein association networks and functional enrichment analyses for any sequenced genome of interest. Nucleic Acids Res. 2023; 51(D1):D638-d646.
^{80.} Jan Kueckelhaus Dieter-Henrik Heiland, Simon Frerich. SPATA2: A Toolbox for Spatial Gene Expression Analysis. https://themilolab.com/.
^{81.} Fadzen C. M., et al. A Platinum(IV) Prodrug-Perfluoroaryl Macrocyclic Peptide Conjugate Enhances Platinum Uptake in the Brain. J Med Chem. 2020; 63(13):6741-6747.
^{82.} Cho C. F., et al. Blood-brain-barrier spheroids as an in vitro screening platform for brain-penetrating agents. Nat Commun. 2017; 8:15623.
^{83.} Fadzen C. M., et al. Perfluoroarene-Based Peptide Macrocycles to Enhance Penetration Across the Blood-Brain Barrier. J Am Chem Soc. 2017; 139(44):15628-15631.
^{84.} lanevski A., et al. SynergyFinder 3.0: an interactive analysis and consensus interpretation of multi-drug synergies across multiple samples. Nucleic Acids Res. 2022; 50(W1):W739-w743.
^{85.} Yadav B., et al. Searching for Drug Synergy in Complex Dose-Response Landscapes Using an Interaction Potency Model. Comput Struct BiotechnolJ. 2015; 13:504-513.
^{86.} Fadzen C. M., et al. Perfluoroarene-Based Peptide Macrocycles to Enhance Penetration Across the Blood-Brain Barrier. J Am Chem Soc. 2017; 139(44):15628-15631.
^{87.} Bergmann S., et al. Blood-brain-barrier organoids for investigating the permeability of CNS therapeutics. Nat Protoc. 2018; 13(12):2827-2843.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

We claim:

1. A method for treating a subject afflicted with a malignant brain tumor, the method comprising administering to the subject:
(i) a chemotherapeutic agent; and
(ii) indirubin or a derivative of indirubin thereof;
wherein the indirubin or a derivative thereof enhances an uptake of the chemotherapeutic agent into brain tumors;
wherein the indirubin or a derivative thereof includes a compound of formula 1: 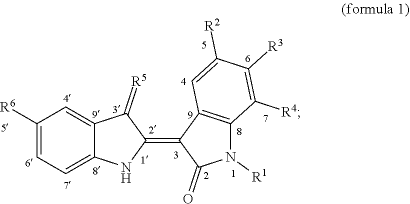
wherein R¹ is selected from —H, —CH₃, and —CH₂CH₃;
wherein R² is selected from —H, —SO₃H, —SO₂NH₂, —OCH₃, —NHCO-tert-Butyl, —NO₂, —NHCOCH(C₆H₅)₂, —NO₂, —Br, —I, —Cl, -and —F;
wherein R³ and R⁴ are each independently selected from —H, —Br, —I, —Cl, -and —F;
wherein R⁵ is selected from ═O, =NOH, =NOCH₃, =NOCOCH₃, =NOCH₂CH₂OCH₂CH₂, and =NOCH₂CH₂CH(OH)CH₂OH;
wherein R⁶ is selected from —H and —COOH.

2. The method of claim 1, wherein the indirubin derivative comprises 6-bromo-indirubin acetoxime (BiA).

3. The method of claim 1, further comprising wherein the indirubin derivative includes:

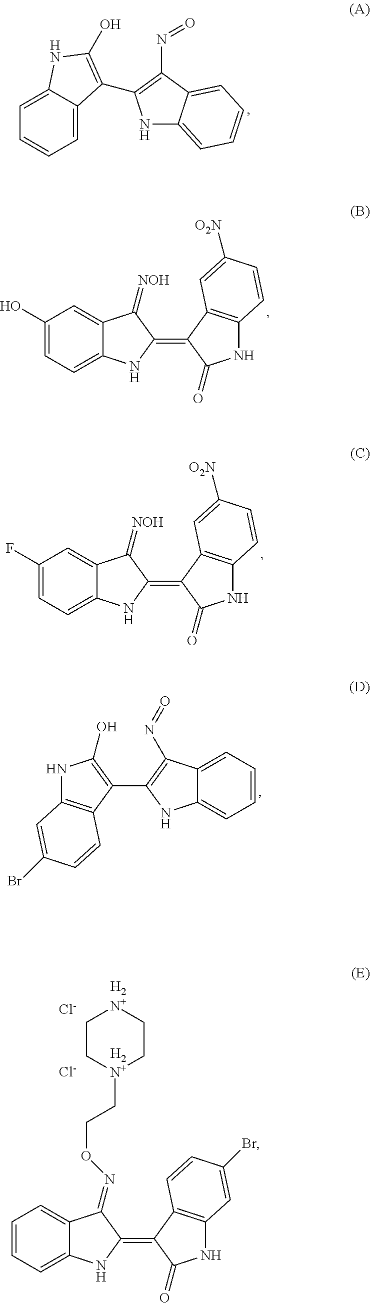

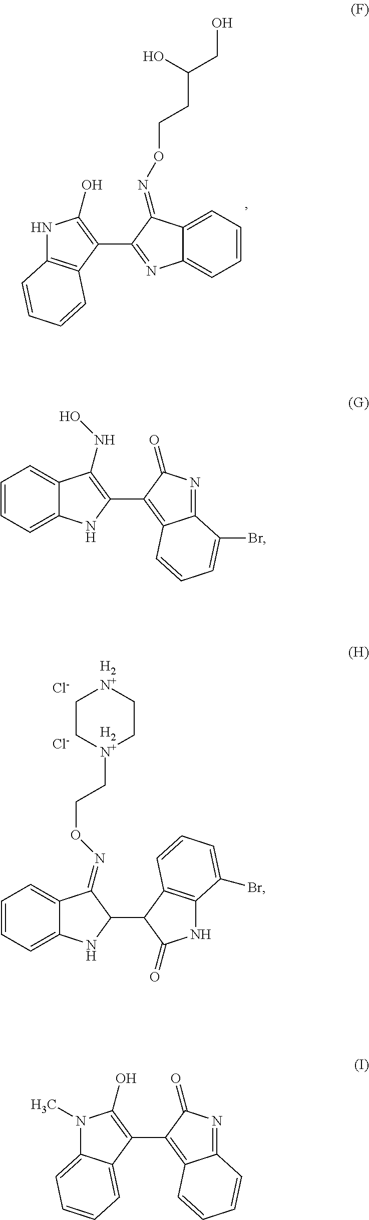

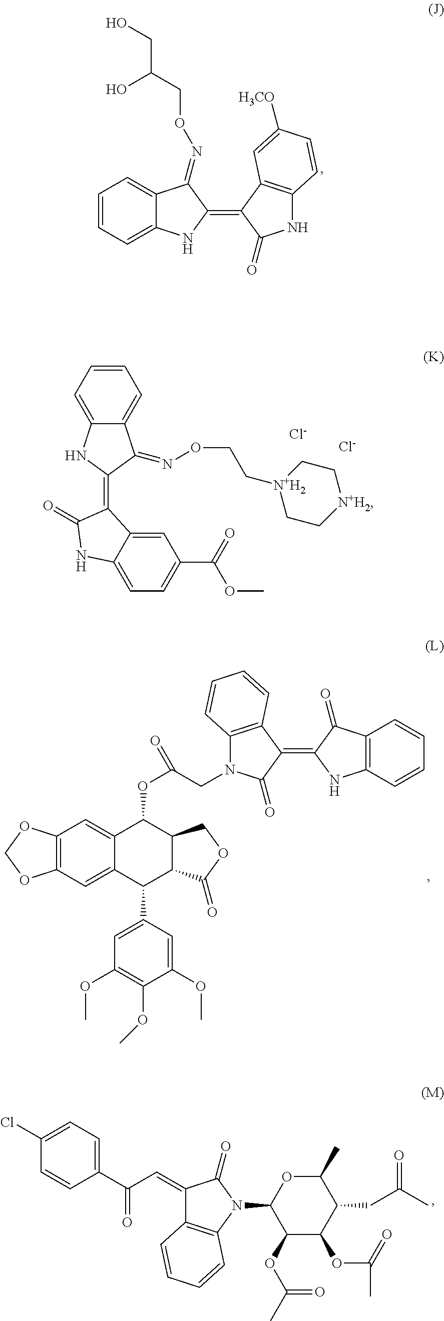

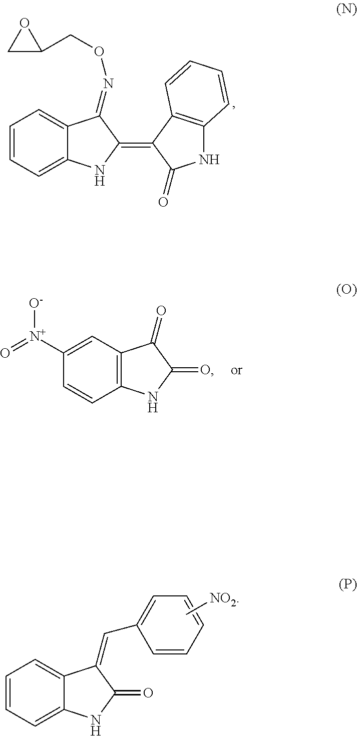

4. The method of claim 1, wherein the chemotherapeutic agent is selected from the group consisting of: carboplatin, carmustine, cisplatin, cyclophosphamide, etoposide, irinotecan, lomustine, methotrexate, procarbazine, temozolomide, and vincristine.

5. The method of claim 1, wherein the chemotherapeutic agent is selected from the group consisting of: a platelet-derived growth factor receptor (PDGFR) inhibitor, a vascular endothelial growth factor (VEGF) inhibitor, a broad-selectivity kinase inhibitor, a PI3K inhibitor, a GSK3 inhibitor, a Src inhibitor, and a Janus kinase inhibitor (JAK inhibitor).

6. The method of claim 5, wherein the PDGFR inhibitor is selected from the group consisting of: Imatinib, Axitinib, Cediranib, Foretinib, Imatinib mesylate, Linifanib, Masitinib, Nintedanib, Ponatinib, Sorafenib, Sunitinib malate, AC 710, AP 24534, CP 673451, DMPQ dihydrochloride, JNJ 10198409, KG 5, PD 166285 dihydrochloride, SU 16f, and SU 6668.

7. The method of claim 5, wherein the VEGF inhibitor is selected from the group consisting of: Votrient (pazopanib), Sutent (sunitinib), Avastin (bevacizumab), Nexavar (sorafenib), Stivarga (regorafenib), Cabometyx (cabozantinib), Lenvima (lenvatinib), Iclusig (ponatinib), Cometriq (cabozantinib), Zaltrap (ziv-aflibercept), Inlyta (axitinib), Zirabev (bevacizumab), Vegzelma (bevacizumab), Mvasi (bevacizumab), Fotivda (tivozanib), Cyramza (ramucirumab), Caprelsa (vandetanib), and Alymsys (bevacizumab).

8. The method of claim 5, wherein the broad-selectivity kinase inhibitor is selected from the group consisting of: bosutinib, crizotinib, dasatinib, erlotinib, gefitinib, lapatinib, pazopanib, ruxolitinib, sunitinib, and vemurafenib.

9. The method of claim 5, wherein the PI3K inhibitor is selected from the group consisting of: Buparlisib (BKM120), Zydelig (idelalisib), Piqray (alpelisib), Vijoice (alpelisib), Copiktra (duvelisib), and Aliqopa (copanlisib).

10. The method of claim 5, wherein the GSK3 inhibitor is selected from the group consisting of: elraglusib, Indirubin-3′-oxime (IDR30, 130), (E/Z)-GSK-3p inhibitor 1, 7-bromoindirubin-3-oxime (7B10), WAY-119064, PF-04802367 (PF-367), RGB-286638 free base, Paeoniae Radix Rubra Extract, Alsterpaullone (Alp, 9-Nitropaullone, NSC 705701), SB216763, Laduviglusib (CHIR-99021, CT99021), AT7519, TWS119, Indirubin (NSC 105327), SB415286, CHIR-98014 (CT98014), Tideglusib (NP031112, NP-12), Laduviglusib (CHIR-99021; CT99021), TDZD-8 (NP 01139), Resibufogenin (Bufogenin, Recibufogenin), 5-Bromoindole, LY2090314, AZD1080, 1-Azakenpaullone (1-Akp), BIO (GSK-3 Inhibitor IX, 6-bromoindirubin-3-oxime, 6-Bromoindirubin-3′-oxime, MLS 2052), AZD2858, AR-A014418, IM-12, Bikinin, BIO-acetoxime, CP21R7 (CP21), 9-ING-41, BRD0705, MAZ51, and Chonglou Saponin VII.

11. The method of claim 5, wherein the Src inhibitor is selected from the group consisting of: Dasatinib, Alsterpaullone, Bosutinib, Herbimycin A, Piceatannol, Saracatinib, Squarunkin A hydrochloride, Tilfrinib, A 419259 trihydrochloride, AZM 475271, JNJ 10198409, KB SRC 4, KX2-391, LCB 03-0110 dihydrochloride, PD 166285 dihydrochloride, PKI 166 hydrochloride, PP1, 1-Naphthyl PP1, PP2, PP3, Src 11, SU 6656, TC-S 7003, TL 0259, and WH-4-023.

12. The method of claim 5, wherein the JAK inhibitor is selected from the group consisting of: Jakafi (ruxolitinib), Cibinqo (abrocitinib), Inrebic (fedratinib), Olumiant (baricitinib), Opzelura (ruxolitinib), Rinvoq (upadacitinib), and Xeljanz (tofacitinib).

13. The method of claim 1, wherein the administration of the chemotherapeutic agent is done concomitantly or sequentially with the administration of indirubin or a derivative thereof.

14. The method of claim 1, wherein the administration of the chemotherapeutic agent and indirubin or a derivative thereof is done concomitantly or sequentially with one or more of a surgical resection, radiotherapy, antiangiogenic therapy, immune therapy, gamma knife radiosurgery, and symptomatic management with corticosteroids.

15. The method of claim 1, wherein the malignant brain tumor is selected from the group consisting of: astrocytoma, glioblastoma, oligodendrocytoma, pilocytic astrocytoma, diffuse intrinsic pontine glioma, ependymoma, oligo-astrocytoma, oligodendrogliocytoma, optic pathway glioma, and hypothalamic glioma.

16. A method for enhancing the uptake of a chemotherapeutic agent into a brain tumor in a subject in need thereof, the method comprising administering to the subject:
(i) a chemotherapeutic agent; and
(ii) indirubin or a derivative of indirubin thereof,
wherein the indirubin or a derivative thereof enhances the uptake of the chemotherapeutic agent into brain tumors.

17. The method of claim 16, wherein the indirubin or a derivative thereof is 6-bromo-indirubin acetoxime (BiA).

18. The method of claim 16, wherein the indirubin or a derivative thereof includes a compound of formula 1: 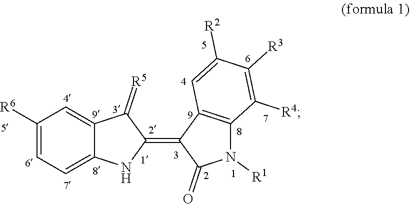
wherein R¹ is selected from —H, —CH₃, and —CH₂CH₃;
wherein R² is selected from —H, —SO₃H, —SO₂NH₂, —OCH₃, —NHCO-tert-Butyl, —NO₂, —NHCOCH(C₆H₅)₂, —NO₂, —Br, —I, —Cl, -and —F;
wherein R³ and R⁴ are each independently selected from —H, —Br, —I, —Cl, -and —F;
wherein R⁵ is selected from =O, =NOH, =NOCH₃, =NOCOCH₃, =NOCH₂CH₂OCH₂CH₂, and =NOCH₂CH₂CH(OH)CH₂OH;
wherein R⁶ is selected from —H and —COOH.

19. The method of claim 16, wherein the indirubin derivative includes: 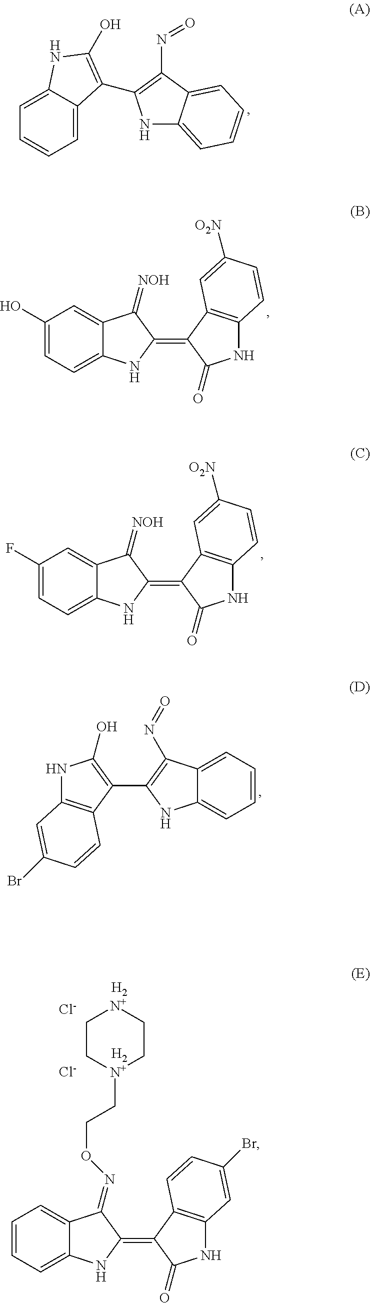 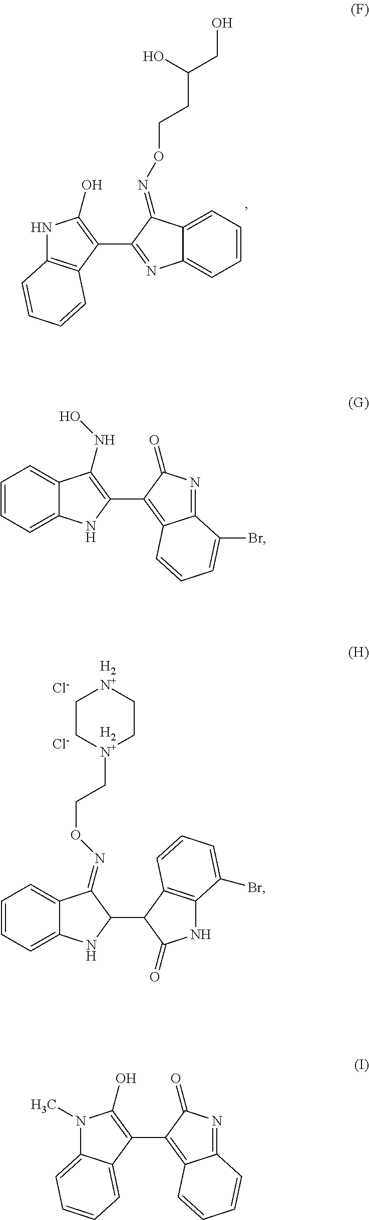 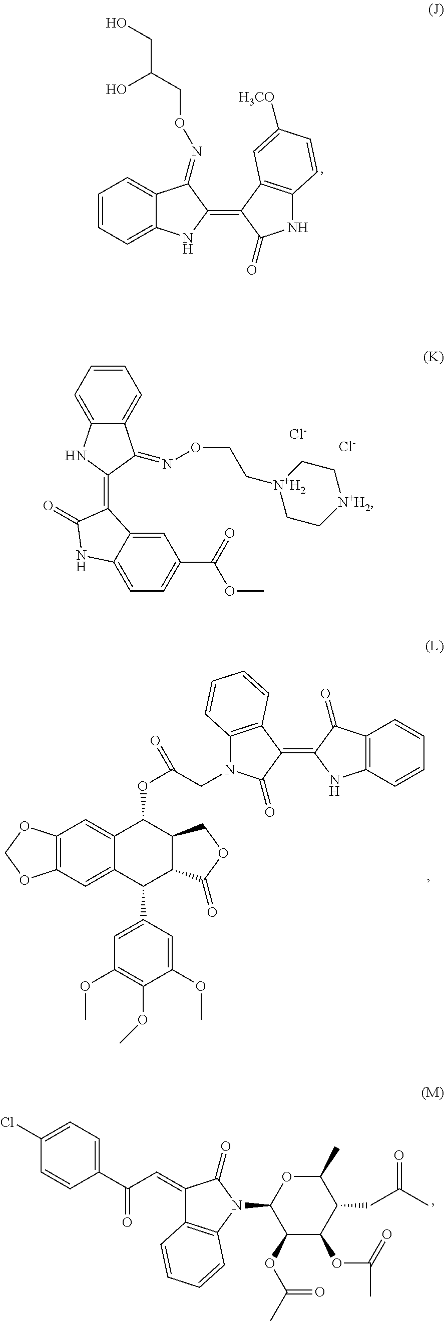 [chemical structure]


SEQUENCE LISTING (8 sequences)

SEQ ID NO: 1 - DNA, 19 bp, synthetic construct
  source 1..19: mol_type = other DNA
ggtcgatgcagagacagga

SEQ ID NO: 2 - DNA, 20 bp, synthetic construct
  source 1..20: mol_type = other DNA
ccagcaagagcacaagagga

SEQ ID NO: 3 - DNA, 20 bp, synthetic construct
  source 1..20: mol_type = other DNA
gactgctctaccatcctgcc

SEQ ID NO: 4 - DNA, 19 bp, synthetic construct
  source 1..19: mol_type = other DNA
gcgctcgtctccgtctatt

SEQ ID NO: 5 - DNA, 19 bp, synthetic construct
  source 1..19: mol_type = other DNA
gagtctccaggtttcgcca

SEQ ID NO: 6 - DNA, 20 bp, synthetic construct
  source 1..20: mol_type = other DNA
acatggcaactgtgaggagg

SEQ ID NO: 7 - DNA, 20 bp, synthetic construct
  source 1..20: mol_type = other DNA
gatgcgtgcgtagaggatca

SEQ ID NO: 8 - DNA, 20 bp, synthetic construct
  source 1..20: mol_type = other DNA
agatgatgttggctcccagg